(19) United States
(12) Patent Application Publication
(10) Pub. No.: US 2011/0306064 A1
(43) Pub. Date: Dec. 15, 2011

(54) Quantifiable Internal Reference Standards For Immunohistochemistry And Uses Thereof

(75) Inventors: Clive R. Taylor, South Pasadena, CA (US); Shan Rong Shi, Los Angeles, CA (US)

(73) Assignee: UNIVERSITY OF SOUTHERN CALIFORNIA

(21) Appl. No.: 13/174,585

(22) Filed: Jun. 30, 2011

Related U.S. Application Data
(63) Continuation-in-part of application No. 11/772,042, filed on Jun. 29, 2007.
(60) Provisional application No. 60/817,969, filed on Jun. 30, 2006.

(51) Int. Cl. G01N 33/53

(52) U.S. Cl. 435/7.21; 435/7.1; 435/7.92

(57) ABSTRACT

Methods for identifying Quantifiable Internal Reference Standards (QIRS) for immunohistochemistry (IHC). Also disclosed are methods for using QIRS to quantify test antigens in IHC.

### CROSS-REFERENCE TO RELATED APPLICATIONS

This application is a continuation-in-part of application Ser. No. 11/772,042, filed Jun. 29, 2007, claims priority to U.S. Provisional Application Ser. No. 60/817,969, filed Jun. 30, 2006, the content of both of which are incorporated herein by reference in their entirety.

### STATEMENT REGARDING FEDERALLY SPONSORED RESEARCH OR DEVELOPMENT

This invention was made with government support under Contract No. DE010861 awarded by the National Institutes of Health. The government has certain rights in the invention.

### FIELD OF THE INVENTION

The present invention relates in general to immunohistochemistry (IHC). More specifically, the invention provides methods for identifying Quantifiable Internal Reference Standards (QIRS) for quantitative analysis of formalin-fixed, paraffin-embedded (FFPE) cell or tissue samples. The invention also provides methods for using QIRS in quantitative analysis of FFPE cell or tissue samples.

### BACKGROUND OF THE INVENTION

Standardization of IHC for archival FFPE tissue sections has become increasingly important due to the emergence of a new field of pathology that requires demonstration of the differential expression of various prognostic markers for individualized cancer treatment. From a practical point of view, one of the most difficult issues in the standardization of IHC for FFPE tissue is the adverse influence of formalin upon antigenicity, and the great variation in fixation/processing procedures.

### SUMMARY OF THE INVENTION

One embodiment of the present invention is directed to a method of quantitatively determining the amount of a test analyte by IHC. The method comprises providing a formalin-fixed, paraffin-embedded (FFPE) cell or tissue sample comprising the test analyte, the FFPE sample having been prepared from an original cell or tissue sample having an original amount the test analyte at a collection time, T₁; identifying a quantifiable internal reference standard (QIRS) for the test analyte, the QIRS being a second analyte present in the original cell or tissue sample at the collection time, T₁, and that is different from the test analyte; providing one or more ratios consisting of the ratio of the amount of the test analyte to the amount of the QIRS in the original cell or tissue sample (A), the ratio of the amount of the test analyte to the amount of the QIRS in the FFPE sample (B), and the ratio of the amount of the QIRS in the original cell or tissue sample to the amount of the QIRS in the FFPE sample (C), said ratios being operable at a test time, T₂, after the collection time; Generating an IHC signal corresponding to amount of QIRS in the test sample at the test time, T2; generating an IHC signal corresponding to amount of test analyte in the test sample at the test time, T2; and calculating at least one of the amount of the test analyte in the test FFPE sample by multiplying the amount of the QIRS in the test FFPE sample by the ratio (B), and the amount of the test analyte in the test original cell or tissue sample by multiplying the amount of the QIRS in the test FFPE sample by the ratio (C) and by the ratio (A).

In another the embodiment of the present invention preferably includes providing a formalin-fixed, paraffin-embedded (FFPE) cell or tissue sample comprising the test analyte, the FFPE sample having been prepared from an original cell or tissue sample having an original amount the test analyte at a collection time, T₁; and identifying a quantifiable internal reference standard (QIRS) for the test analyte, the QIRS being a second analyte present in the original cell or tissue sample at the collection time, T₁, and that is different from the test analyte; providing a reference calibration curve indicating at least a ratio of the amount of the test antigen to the amount of the QIRS in a reference FFPE sample at test times, T₂, after T₁; measuring a first IHC signal corresponding to the amount of the QIRS in the FFPE sample at test time T₂, wherein the first IHC signal varies depending on at least the concentration of the QIRS; measuring a second IHC signal corresponding to the amount of the test analyte in the FFPE sample at time T₂, wherein the second IHC signal varies depending on at least the concentration of the test analyte; and applying the calibration curve to the first IHC signal and the second IHC signal of the test antigen in the FFPE sample to determine the amount of the test antigen in the FFPE sample. Preferably, the calibration curve provides a ratio, A, of the original amount of the test analyte to the original amount of the QIRS and a ratio, C, of the original amount of the QIRS to the amount of the QIRS in the FFPE sample at time T₂ is known, and the amount the test analyte in the original sample is calculated by multiplying the amount QIRS in the FFPE sample by the ratio A and by the Ratio C.

In a preferred embodiment, the original cell is an endothelial cell or the original tissue contains endothelial cells, or the original cell is a lymphocyte or the tissue contains lymphocytes, or the original cell is a mesenchymal or epithelial cell, or the original tissue contains mesenchymal or epithelial cells. The QIRS is a cell surface protein, a cytoplasmic protein, or a nuclear protein. The method of claim 2, wherein the QIRS is a cell surface protein, a cytoplasmic protein, or a nuclear protein. The QIRS is more preferably selected from the group consisting of CD31, actin, B2 microglobulin, vimentin, factor VIII, histone H1, MIB1, Fli 1, CD34, and VWF.

Another embodiment of the present invention is directed to a method for identifying a QIRS for IHC. The method comprises the steps of (1) providing multiple samples of cells or tissues of the same type or different types, (2) determining the amount of a first antigen (the QIRS) and the amount of a second antigen (the test antigen or analyte) in each of the cell or tissue samples, (3) preparing an FFPE sample from each of the cell or tissue samples, and (4) determining the amount of the first antigen (QIRS) and the amount of the second antigen (test antigen) in each of the FFPE samples by IHC. If the ratio of the amount of the first antigen to the amount of the second antigen in the cell or tissue samples is at least 95% identical among the cell or tissue samples and the ratio of the amount of the first antigen to the amount of the second antigen in the FFPE samples is at least 95% identical among the FFPE samples, the first antigen is identified as a QIRS for the second antigen in IHC. Preferably, the amount of the first antigen in the FFPE samples is at least 50% of the amount of the first antigen in the cell or tissue samples. The amount of the first antigen (the QIRS) in the FFPE sample may be determined using a first quantifiable label and the amount of the second antigen (the test antigen) in the FFPE sample may be determined using a second quantifiable label. In some embodiments, the first antigen (QIRS) is detectable by a first antibody to the first antigen or the second antigen (test antigen) is detectable by a second antibody to the second antigen.

Another embodiment of the present invention is directed a method for quantifying a test analyte by IHC. The method comprises the steps of (1) providing an FFPE cell or tissue sample prepared from an original cell or tissue sample, (2) determining the amount of a QIRS for a test antigen in the FFPE sample by IHC, and (3) calculating the amount of the test antigen (analyte to be measured) in the FFPE sample from the amount of the QIRS in the FFPE sample. The method may further comprise a step of calculating the amount of the test antigen in the original cell or tissue sample from the amount of the QIRS in the FFPE sample. The QIRS may be identified according to the method described above.

Normal or pathologic cells or tissues may be used to practice the methods of the invention. For example, the cells may be in the form of cell lines, such as lymphocytes (e.g., Raji or HL60 cells), endothelial cells (e.g., HuVEC cells), fibroblasts (e.g., LD419 cells), or epithelial cells (e.g., breast cells such as MCF7, MDA, or MB468 cells), or the cells may be in normal or pathologic tissues which may contain lymphocytes, endothelial cells, fibroblasts, or epithelial cells. Alternatively, the cells or tissues may be from prostate or spleen.

A QIRS may be a cell surface protein, a cytoplasmic protein, or a nuclear protein. Exemplary QIRS include but are not limited to PSA, p53, Rb, and ER. In particular, exemplary QIRS for lymphocytes include but are not limited to CD45, CD20, actin, B2 microglobulin, vimentin, histone H1, and MIB1; exemplary QIRS for endothelial cells include but are not limited to CD31, actin, B2 microglobulin, vimentin, factor VIII, histone H1, MIB1, Fli 1, CD34, and VWF; exemplary QIRS for fibroblasts include but are not limited to fibroblast surface protein, actin, B2 microglobulin, vimentin, desmin, histone H1, and MIB1; and exemplary QIRS for epithelial cells include but are not limited to Her2, EGFR, actin, B2 microglobulin, vimentin, histone H1, and MIB1.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1. In stained tissues two or more colors occurring together must be separated for quantification. Figure shows unmixing of DAB from hematoxylin: Ki67 in a lymph node germinal center imaged with a Nuance multispectral imaging system. Panel A: visual (RGB) appearance of the sample. Pane B: unmixed DAB signal. Panel C: unmixed hematoxylin signal, which accurately recapitulates the dense staining of the mantle cells and the paler staining of the germinal center. The small box indicates the region highlighted in FIG. 2.

FIG. 2. Unmixing of DAB from hematoxylin: Choice of DAB spectrum affects quantitative results. Differing spectra for the DAB (along with a constant hematoxylin spectrum) are shown in Panels A, D and G, and the respective unmixing results are shown in the corresponding rows. The unmixed hematoxylin channels are shown in the second column and the combined DAB plus hematoxylin result is shown in the third column. The numeric values shown represent the integrated optical density of the DAB signal from the circled nucleus. The third row represents the best DAB spectral estimate, with hematoxylin values for Ki67(+) and (−) nuclei displaying similar intensities. See text for additional discussion.

FIG. 3. Three-color unmixing of plastic films with spectra similar to brown and red IHC chromogens and hematoxylin. The strips were arranged so that single, double and triple overlapping regions were present (representative regions are indicate by numbers in Panel A). A spectral data set was acquired; spectra corresponding to the individual plastic strip species are shown in Panel B. Using these spectra, the image cube was unmixed to create individual images of each colored strip by itself (colored in the pseudocolors of the spectral library used for unmixing). Intensity profiles are shown for each strip, indicating that quantitative unmixing could be achieved even when 2 or 3 absorbing species spatially overlapped.

FIG. 4. Detection and unmixing of ER-(DAB) and PR-(Vulcan Red) signals from a breast tissue specimen counterstained with hematoxylin. The 6 panels illustrate the original visual appearance (A), and after unmixing, the H channel (B, which can be used to identify the nuclear compartment for quantitative purposes), and separate channels for ER (C) and PR (D). The dotted oval identifies a region of presumptively normal epithelium, and the red oval a region of invasive ductal carcinoma. The bottom panels show an overlay of the green and red channels (E), and finally, a depiction of the original image with ER-PR double-positive cells indicated using a yellow mask (F).

FIG. 5. A. Double IHC stain for ER (DAB-brown) and PR (FAST RED), plus hematoxylin (blue)—cannot be read with naked eye. B. Spectral analysis (unmixing) clearly separates stains; allows comparison and measurement of intensity of peak colors.

FIG. 6. Triple IHC stain—epithelial cells (brown), Kappa cells (blue), lambda cells (red), showing power of spectral unmixing which allows comparison of intensity of the peak pure colors. By these means test analytes could be measured against a calibrated reference protein (the QIRS).

FIG. 7. FIG. 7(A) show staining for immunoglobulin epitopes (antigens) in fixed sections, specifically looking at plasma cells for kappa and lambda (K and L) light chains. The approach is based on the reciprocal and exclusive distribution of K and L. A plasma cell contains either K or L,—never both. Therefore a double stain for K and L will show a K population and a L population, with no overlap, each stain confirming therefore the performance and specificity of the other (in fact each serving as an ‘internal reference standard’ for the other qualitatively). The K land L stain is purely qualitative and does not have all the required characteristics for quantification, whereas internal reference standards (QIRS), by definition, having been quantified, do meet these criteria.

FIG. 8. An internal reference standard must survive fixation and processing and must be widely and uniformly present in tissues that are to be tested (infinite supply, inbuilt negative positive etc). This slide shows a panel of candidate QURS proteins, and their internal tissue locations These proteins have been tested as candidate QIRS analytes. Each of these proteins was stained by IHC in tissues subjected to wide ranges of different fixation and processing procedures (to show presence in routine tissue sections) and then analyzed for degree of degradation by fixation and for widespread and consistent distribution in tissues. Typical examples of the results are shown in FIGS. 10, 11, 12 and 13.

FIG. 9 shows the experimental design of a study of candidate QIRS analytes using tissue microarrays (TMAs) prepared to contain 120 to 180 2 mm cores from FFPE* tissues, each in triplicate (thus representing 40 to 60 tissue samples fixed for known time differences). (*FFPE formalin fixed paraffin embedded, the routine method in use today). A. Parallel cuts of TMA slide are then stained with up to 20 antibodies, eg 2 antibodies for each of the 10 candidate QIRS proteins, or 10 or more different candidate proteins. B. The patterns and intensity of IHC staining are scored visually and by image analysis (in this example using the Chromavision ACTS system, but CRI Nuance spectral imaging software also has been used. C. Intensities are compared for different fixation times to establish the performance of candidate QIRS proteins under differing conditions.

FIG. 10 shows a representative study of two candidate QIRS anayltyes (proteins), vimentin (10B) and Histone H1 (A) in pig tissues, that had been taken fresh, cut into small tissue blocks and then fixed in formalin for times ranging from 3 hrs to 7 days. Prior to staining for vimentin or H1, tissues were either subjected to antigen retrieval (AR) in order to try to recover any protein lost under fixation, or not. These were done in parallel and results compared for intensity of stain under differing fixation conditions. All candidate QIRS proteins were evaluated by similar methods in porcine tissues in order to develop data as to the rate of degradation (or loss) of each protein during exposure to fixative. (+++/++/+/− represents ‘semi-quantitative’ scoring of intensity of stain performed visually, with +++ being the highest intensity.

FIG. 11 shows a progressive loss of intensity of staining of desmin (a candidate QIRS protein) in myocardium, fixed for 6, 24 and 72 hrs, and for 30 days. Data from sequential studies are then assembled to develop a standard degradation of loss curve for desmin, and each other candidate QIRS protein. Intensity was measured visually using semi quantitative scoring ((+++/++/+/− scale) and sample curves are shown in slide 13.

FIG. 12 shows the progressive loss of intensity of staining of caldesmon (another candidate QIRS protein) in lung, fixed for 6, 24, 48 and 72 hrs, and for 7 and 30 days. Data from sequential studies are then assembled to develop a standard degradation of loss curve for caldesmon, and each other candidate QIRS protein. Intensity was measured visually using semi quantitative scoring and sample curves are shown in slide 13.

FIG. 13 shows representative degradation or loss curves for 10 candidate QIRS proteins developed by the studies described in prior slides, over a fixation period of up to 5+ days. This study shows results with AR (antigen retrieval) when some proteins show little degradation, and others show a consistent fall. With known data for any QIRS protein a standard degradation curve can be constructed and then used as a calibration curve to evaluate the intensity of staining of ‘test’ proteins (analytes) in an IHC assay. Providing that the degradation curve of the ‘test’ protein or analyte previously has also been established by similar methods, then the intensity of IHC staining for the test protein can be compared to the intensity of the QIRS, providing a basis for calculating the amount of test protein present (quantification).

### DETAILED DESCRIPTION OF THE INVENTION

### Abbreviations

QIRS=Quantifiable Internal Reference Standard

FFPE=formalin-fixed, paraffin-embedded

IHC=Immunohistochemistry

AR=Antigen Retrieval

### DEFINITIONS

As used herein, the term “antigen” refers to any substance capable of eliciting an immune response in a subject. Exemplary antigens include but are not limited to peptides, proteins, lipoproteins, and glycoproteins. The amount of an antigen in a cell or tissue sample may be determined by methods commonly known in the art. For example, methods of measuring protein levels in biological samples usually employ antibodies (e.g., monoclonal or polyclonal antibodies) that bind specifically to target proteins. The term “antibody” refers to immunoglobulin molecules and immunologically active portions thereof, i.e., molecules that contain an antigen binding site which specifically binds an antigen. Examples of immunologically active portions of immunoglobulin molecules include F(ab) and F(ab′)₂ fragments which can be generated by treating the antibody with an enzyme such as pepsin. Alternatively, antigens may be detected by aptamers, which are chemically synthesized (usually short) strands of oligonucleotides (DNA or RNA) that can adopt highly specific three-dimensional conformations.

As used herein, the term “subject” refers to a human or animal, including all mammals such as primates (particularly higher primates), sheep, dog, rodents (e.g., mouse or rat), guinea pig, goat, pig, cat, rabbit, and cow. In a preferred embodiment, the subject is a human.

A “tissue” as used herein refers to is a cellular organizational level intermediate between cells and a complete organism. A tissue is an ensemble of cells, not necessarily identical, but from the same origin. Exemplary tissues include, but are not limited to, epithelial, connective, muscle, nervous, heart, lung, brain, eye, stomach, spleen, bone, pancreatic, kidney, gastrointestinal, skin, uterus, thymus, lymph node, colon, breast, prostate, ovarian, esophageal, head, neck, rectal, testis, throat, thyroid, intestinal, melanocytic, colorectal, liver, gastric, and bladder tissues. Cells may be obtained, e.g., from cell culture or breakdown of tissues. A tissue sample from a subject may include, but is not limited to, a biopsy specimen sample, a normal or benign tissue sample, a cancer or tumor tissue sample, a freshly prepared tissue sample, a frozen tissue sample, a primary cancer or tumor sample, or a metastasis sample.

One embodiment of the present invention is directed to a method of quantitatively determining the amount of a test analyte by IHC. The method comprises providing a formalin-fixed, paraffin-embedded (FFPE) cell or tissue sample comprising the test analyte, the FFPE sample having been prepared from an original cell or tissue sample having an original amount the test analyte at a collection time, T₁; identifying a quantifiable internal reference standard (QIRS) for the test analyte, the QIRS being a second analyte present in the original cell or tissue sample at the collection time, T₁, and that is different from the test analyte; providing one or more ratios consisting of the ratio of the amount of the test analyte to the amount of the QIRS in the original cell or tissue sample (A), the ratio of the amount of the test analyte to the amount of the QIRS in the FFPE sample (B), and the ratio of the amount of the QIRS in the original cell or tissue sample to the amount of the QIRS in the FFPE sample (C), said ratios being operable at a test time, T₂, after the collection time; Generating an IHC signal corresponding to amount of QIRS in the test sample at the test time, T2; generating an IHC signal corresponding to amount of test analyte in the test sample at the test time, T2; and calculating at least one of the amount of the test analyte in the test FFPE sample by multiplying the amount of the QIRS in the test FFPE sample by the ratio (B), and the amount of the test analyte in the test original cell or tissue sample by multiplying the amount of the QIRS in the test FFPE sample by the ratio (C) and by the ratio (A).

In another the embodiment of the present invention preferably includes providing a formalin-fixed, paraffin-embedded (FFPE) cell or tissue sample comprising the test analyte, the FFPE sample having been prepared from an original cell or tissue sample having an original amount the test analyte at a collection time, T₁; and identifying a quantifiable internal reference standard (QIRS) for the test analyte, the QIRS being a second analyte present in the original cell or tissue sample at the collection time, T₁, and that is different from the test analyte; providing a reference calibration curve indicating at least a ratio of the amount of the test antigen to the amount of the QIRS in a reference FFPE sample at test times, T₂, after T₁; measuring a first IHC signal corresponding to the amount of the QIRS in the FFPE sample at test time T₂, wherein the first IHC signal varies depending on at least the concentration of the QIRS; measuring a second IHC signal corresponding to the amount of the test analyte in the FFPE sample at time T₂, wherein the second IHC signal varies depending on at least the concentration of the test analyte; and applying the calibration curve to the first IHC signal and the second IHC signal of the test antigen in the FFPE sample to determine the amount of the test antigen in the FFPE sample. Preferably, the calibration curve provides a ratio, A, of the original amount of the test analyte to the original amount of the QIRS and a ratio, C, of the original amount of the QIRS to the amount of the QIRS in the FFPE sample at time T₂ is known, and the amount the test analyte in the original sample is calculated by multiplying the amount QIRS in the FFPE sample by the ratio A and by the Ratio C.

In a preferred embodiment, the original cell is an endothelial cell or the original tissue contains endothelial cells, or the original cell is a lymphocyte or the tissue contains lymphocytes, or the original cell is a mesenchymal or epithelial cell, or the original tissue contains mesenchymal or epithelial cells. The QIRS is a cell surface protein, a cytoplasmic protein, or a nuclear protein. The method of claim 2, wherein the QIRS is a cell surface protein, a cytoplasmic protein, or a nuclear protein. The QIRS is more preferably selected from the group consisting of CD31, actin, B2 microglobulin, vimentin, factor VIII, histone H1, MIB1, Fli 1, CD34, and VWF.

Use of the QIRS in connection with IHC, including IHC staining protocols, provide quality control for the entire staining process and may be thought of as analogous to the standardized reference materials used in clinical laboratory testing, of blood or serum, where the well characterized reference standard serves as a calibration marker that allows for the precise measurement by weight of an analyte present in unknown amounts.

### Formalin-Fixed Paraffin-Embedded Cell or Tissue Samples

The methods of providing a formalin-fixed, paraffin-embedded (FFPE) cell or tissue sample comprising the test analyte, the FFPE sample having been prepared from an original cell or tissue sample having an original amount the test analyte at a collection time, T₁.

A test analyte according to the present invention is generally defined as the substance or chemical constituent of the FFPE sample that is to be measure in accordance with the present invention. The test analyte is preferably an antigen. In a preferred embodiment, the test analyte is a polypeptide or a protein, such as a lipoproteins or a glycoproteins. In another embodiment of the present invention, the test analyte is a ribonucleic acid or deoxyribonucletide, including an RNA or DNA or fragment thereof.

The test analyte according to the present invention is an intrinsic component of the original cell or tissue sample that is present in the sample at the time the sample is collected. Preferably, the test analyte is intrinsically present in variable amounts in the tissue and requires a quantitative analysis for therapeutic decisions (diagnosis or prognosis), are then subjected to the identical process under controlled conditions.

One object of the present invention is to quantitatively determine an amount of the test analyte at the time the tissue is collected, i.e. a collection time. The collection time is preferably the time at which the sample is first collected, for example, the time at which a sample is removed from an organism. However, the collection time may be also be defined as the time just prior to when sample preparation starts. When defined this way, one object of the present invention would be to determine the amount of the test analyte just prior to sample preparation. The collection time may also be defined, for instance, as the time just after sample preparation. When understood in this manner, one aspect of the present invention is the ability to quantitatively determine the amount of a test analyte at any time prior to the time the test analyte is examined.

Tissues may be obtained from a subject using any of the methods known in the art. In another embodiment, the subject is an experimental animal or animal suitable as a disease model. A “tissue” sample from a subject may be a biopsy specimen sample, a normal or benign tissue sample, a cancer or tumor tissue sample, a freshly prepared tissue sample, a frozen tissue sample, a primary cancer or tumor sample, or a metastasis sample. Exemplary tissues include, but are not limited to, epithelial, connective, muscle, nervous, heart, lung, brain, eye, stomach, spleen, bone, pancreatic, kidney, gastrointestinal, skin, uterus, thymus, lymph node, colon, breast, prostate, ovarian, esophageal, head, neck, rectal, testis, throat, thyroid, intestinal, melanocytic, colorectal, liver, gastric, and bladder tissues. Cells may be obtained, e.g., from cell culture or breakdown of tissues.

The methods of the present invention preferably include providing a formalin-fixed, paraffin-embedded (FFPE) cell or tissue sample.

The types of cells or tissue sample useable in connection with the present invention is not particularly limited. Preferably, the tissues or cells are eukaryotic tissues or cells, preferably mammalian tissues and/or cells and even more preferably human tissue or cells. Normal or pathologic cells or tissues may be used to practice the methods of the invention. For example, the cells may be in the form of cell lines, such as lymphocytes (e.g., Raji or HL60 cells), endothelial cells (e.g., HuVEC cells), fibroblasts (e.g., LD419 cells), or epithelial cells (e.g., breast cells such as MCF7, MDA, or MB468 cells), or the cells may be in normal or pathologic tissues which may contain lymphocytes, endothelial cells, fibroblasts, or epithelial cells. In another embodiment, the cells or tissues may be from prostate or spleen.

The provision of the FFPE sample is usually preceded by preparation of the FFPE cell or tissue sample. The FFPE cell or tissues sample may be prepared according to the FFPE fixation and embedding techniques commonly known to those of ordinary skill. Typically, sections of paraffin-embedded cells or tissues are obtained by (1) preserving tissue in fixative, (2) dehydrating the fixed tissue, (3) infiltrating the tissue with fixative, (4) orienting the tissue such that the cut surface accurately represents the tissue, (5) embedding the tissue in paraffin (making a paraffin block), (6) cutting tissue paraffin block with microtome in sections of 4-5 μm, and (7) mounting sections onto slides.

In an exemplary procedure, specimens used in connection with the present invention may be obtained, for instance, by fine-needle aspiration, or from the operating room by biopsy, or by more extensive therapeutic surgical procedures. Following removal of the tissue from the body, autolysis may generally be arrested by immersion in a fixative. Preferably, the fixative is formalin (in common practice a 4% solution of formaldehyde). Other fixatives may be employed. However, in a preferred embodiment, Formalin is used because it is well known to those of ordinary skill, has a long tradition of use and generally yields sufficient morphologic detail. Formalin also is inexpensive, easily stored, and universally available.

Preferably, excised tissue samples are placed directly in formalin for subsequent transportation to a suitable laboratory. Once at the suitable laboratory, for instance a surgical pathology suite (“grossing” room), the sample may be further cut, meaning that if not already sufficiently small, it is cut into small blocks to facilitate rapid penetration by the fixative (formalin penetrates relatively slowly), and placed in fresh fixative for further processing. In a preferred embodiment, time for fixation of a 5-mm-thick tissue block is about 12-24 hours, the total time in fixative may vary, due to differing transportation times to the laboratory and accumulation of specimens for batch processing. Fixation time, for instance, may vary anywhere from 6-24 hours, or more.

In addition, the formalin which serves as the basis of the fixation process, may affect cell or tissue samples depending upon whether the formalin was freshly prepared and adequately buffered. There is also some variability in the rate of penetration of formalin in different types of tissues and into differently sized blocks.

Following fixation, the sample preparation preferably includes one or more process selected from embedding the tissue or cell in paraffin, de-paraffinization of the cut sections, also exposing the tissues (and therefore the analytes) to a series of chemicals and to heat. The end-result of the process of fixation, embedding and de-parrainization is a formalin-fixed paraffin embedded (FFPE) tissue section.

In a preferred embodiment of the present invention, FFPE samples used in connection with the present invention are subject to the same, or nearly the same, sample preparation methods. In an especially preferred embodiment, test FFPE samples containing the test analyte to be analyzed are prepared using the sample preparation methods used to generate the calibration curves associated with the Quantifiable Internal Reference Standard as described herein.

Preferably, the methods of the present invention include consistent sectioning procedure. For routine staining a precision microtome is used to achieve a section thickness of about 5 μm. A nucleus that is 5 μm in diameter may thus be entirely within the plane of the section, or only partially included, with effects upon the apparent intensity of a nuclear IHC stain, all other things being equal. Thicker sections may manifest the same problem even for quite large nuclei, whereas generally thinner sections will minimize it. Uniform preparation of FFPE sections that are less than 5 μm in thickness may be achieved, for instance, by plastic embedding media, or other special media. Generally, all paraffin embedded sections are floated on a warm water bath (45° C.) before being picked up onto microscope slides and allowed to drain.

Identifying and Validating a Quantifiable Internal Reference Standard

The methods of the present invention include identifying a quantifiable internal reference standard (QIRS) for the test analyte, the QIRS being a second analyte present in the original cell or tissue sample at the collection time, T₁, and that is different from the test analyte. The QIRS is also present intrinsically within original sample or tissues but is different from the test analyte, and the amount of the QIRS in the sample is substantially uniform in the relevant population (preferably less than 10% different across all tested tissue samples). Preferably, the QIRS is common to all (almost) tissue types and is preferably a QIRS for a number of test analytes. In a preferred embodiment of the present invention, a QIRS for a test analyte is an analyte for which the ratio of the amount of the QIRS analyte to the amount of the test analyte in the cell or tissue samples both before and after the FFPE process is suitably consistent among tissue sample before and after the FFPE process as described herein.

The methods of the present invention preferably include methods of identifying and validating an analyte as a QIRS for a test analyte. The method preferably includes identifying a panel of candidate QIRS analytes that are selected on the basis of their presence in relatively constant amounts in specific cell types that are easily recognized and widely distributed (such as endothelial cells or lymphocytes). A QIRS may be a cell surface protein, a cytoplasmic protein, or a nuclear protein. Exemplary QIRS candidates include but are not limited to PSA, p53, Rb, and ER. In particular, an exemplary panel of candidate QIRS analytes for lymphocytes include but are not limited to CD45, CD20, actin, B2 microglobulin, vimentin, histone H1, and MIB1; exemplary QIRS for endothelial cells include but are not limited to CD31, actin, B2 microglobulin, vimentin, factor VIII, histone H1, MIB1, Fli 1, CD34, and VWF; an exemplary panel of candidate QIRS analytes for fibroblasts include but are not limited to fibroblast surface protein, actin, B2 microglobulin, vimentin, desmin, histone H1, and MIB1; and exemplary panel of QIRS candidate for epithelial cells include but are not limited to Her2, EGFR, actin, B2 microglobulin, vimentin, histone H1, and MIB1.

The method identifying and validating a QIRS involves providing multiple samples of cells or tissues having the test analyte and the candidate QIRS analyte selected from the panel of candidate QIRS analytes. The method includes determining the amount of a candidate QIRS analyte and the amount of the test analyte in each of the cell or tissue samples, preparing an FFPE sample from each of the cell or tissue samples, and determining the amount of the QIRS candidate analyte and the amount of the test analyte in each of the FFPE samples by IHC. In validating a QIRS for test analyte, the amount of candidate QIRS analyte present on a per cell basis (averaged across 100 or 1000 cells) is measured experimentally and quantitatively by independent techniques known to those of ordinary skill, such as ELISA (enzyme linked immunosorbent assay) assay of extracts containing known numbers of the critical cell type (that contains the protein). In one embodiment of the present invention, A QIRS for a test analyte is identified by comparing the ratio of the amount of the candidate QIRS analyte to the amount of the test analyte in the cell or tissue samples at a collection time and a test time (e.g. both before and after the FFPE process) as measured by the independent technique. If both ratios are consistent (e.g., at 90% identical and preferably at least 95% identical) among all samples before and after the FFPE process (i.e at the collection and test time), respectively, the first antigen is identified as a suitable QIRS for the second antigen in IHC.

In another embodiment of the present invention, a QIRS for the test analyte is identified by comparing the ratio of the amount of the candidate QIRS analyte to the amount of the test analyte in the cell or tissue samples at a collection time (e.g. before the FFPE process) and a test time after the collection time (e.g. after the FFPE process). In this embodiment, the original cell or tissue samples (e.g., before the FFPE process) and the later samples (i.e., after the FFPE process) may be prepared by different people, at different times, in different labs, or following different procedures. If both ratios are consistent (e.g., at least 90% identical and preferably 95% identical) among all samples before and after the FFPE process (i.e. at the collection time and test time), respectively, the candidate QIRS analyte is identified as a QIRS for the test analyte in IHC. The ratios of any member of the group consisting of (1) the amount of the QIRS in the original cell or tissue sample, (2) the amount of the second antigen in the original cell or tissue sample, (3) the amount of the QIRS in the FFPE sample, and (4) the amount of the second antigen in the FFPE sample to another member of the group may be referred to as “ratios” or alternatively “standard ratios.” These data may also be displayed in the form of a ‘degradation’ or ‘antigen loss’ curve, as for instance, a function of time and/or concentration. The resulting degradation curve for the QIRS then serves as a calibration curve against which to measure the test antigen (analyte) as described herein. The calibration curve indicates at least a ratio of the amount of the test antigen to the amount of the QIRS in a reference FFPE sample at test times, T₂, after T₁ and preferably a ratio, A, of the original amount of the test analyte to the original amount of the QIRS and a ratio, C, of the original amount of the QIRS to the amount of the QIRS in the FFPE sample at time T₂.

The step of identifying and validating the QIRS for the test analyte provides providing one or more ratios consisting of the ratio of the amount of the test analyte to the amount of the QIRS in the original cell or tissue sample (A), the ratio of the amount of the test analyte to the amount of the QIRS in the FFPE sample (B), and the ratio of the amount of the QIRS in the original cell or tissue sample to the amount of the QIRS in the FFPE sample (C), said ratios being operable at a test time, T₂, after the collection time. The ratios of the present invention are operable at the test time, T2, if the validation of the QIRS included validation at test times T2, or if the data can be obtained from calibration curve against which to measure the test antigen (analyte) as described herein.

Since at least one aspect of the present invention involves the identification and validation of candidate QIRS analytes, this aspect may be understand as an investigation tool, or process, whereby candidate QIRS analytes, which are preferably ubiquitous proteins, are identified as being present within recognizable cells in surgical biopsy tissues and are validated (precisely measured by weight) in order that they may serve as a QIRS for test analytes.

The QIRS analytes most preferably meet two critical requirements for a quantitative assay:

1. measurement of the absolute amount of the QIRS after processing of the tissue sample (FFPE) allows for calculation of loss of test analytes that occurs at time, preferably critical times, after initial collection, for example after sample preparation (with reference to the amount originally present in fresh tissue), and

2. measurement of the intensity of the IHC stain reaction of the QIRS as compared to the intensity of reaction for a protein of interest (test analyte), permits quantification of the test analyte that is present in unknown amounts.

### Quantifying the Test Analyte in the FFPE Sample by IHC

The QIRS validated in accordance with the present invention may be used in accordance with the present invention to directly quantify test analytes by, for instance, immunohistochemistry.

The methods of the present invention generally require (1) generating an IHC signal corresponding to amount of QIRS in the test sample at the test time, T2, and (2) generating an IHC signal corresponding to amount of test analyte in the test sample at the test time, T2. Preferably, the IHC signal is proportional to the amount (or concentration) of both the QIRS and the test analyte in the FFPE sample. Since the QIRS is generally present in known and relatively constant amounts in cells in tissues, the known concentration may be used to relate a particular intensity (i.e. an IHC signal) from an IHC stain at a test time can to the intensity (i.e. amount) the signal WOULD HAVE BEEN IN FRESH TISSUE, and therefore the loss of the can be derived (the IHC signal may “roughly” be thought sort of a surrogate data point for fixation time and fixation loss). However, the test antigen is present in variable amounts in different tissues/cells and although its degradation curve is known, when a particular intensity is seen it cannot be determined where it lies along the curve and which curve it lies on—because different tissues with different amounts of test antigen in the fresh state will each generate a different start point for the calibration curve. In simple terms, the intensity of IHC stain reaction of the recognizable cell type (that contains ubiquitous characterized reference standard protein, i.e., the QIRS), is compared with the intensity of IHC stain of the cell(s) containing the ‘test analyte’. Because the amount of QIRS can be measured accurately, using the data derived in establishing the QIRS, the amount present of the test analyte can be calculated.

One embodiment of the present invention includes generating an IHC signal corresponding to amount of QIRS in the test sample at the test time, T2, generating an IHC signal corresponding to amount of test analyte in the test sample at the test time, T2; and calculating at least one of the amount of the test analyte in the test FFPE sample by multiplying the amount of the QIRS in the test FFPE sample by the ratio (B), and the amount of the test analyte in the test original cell or tissue sample by multiplying the amount of the QIRS in the test FFPE sample by the ratio (C) and by the ratio (A). For example, when the standard ratios of the amount of the test antigen to the amount of the QIRS in the original cell or tissue sample (A), the amount of the test antigen to the amount of the QIRS in the FFPE sample (B), and the amount of the QIRS in the original cell or tissue sample to the amount of the QIRS in the FFPE sample (C) are known, the amount of the test antigen in the test FFPE sample may be calculated as [the amount of the QIRS in the test FFPE sample]×(B), and the amount of the test antigen in the test original cell or tissue sample may be calculated as [the amount of the QIRS in the test FFPE sample]×(C)×(A).

Another method of the present invention includes measuring a first IHC signal corresponding to the amount of the QIRS in the FFPE sample at test time T₂, wherein the first IHC signal varies depending on at least the concentration of the QIRS, measuring a second IHC signal corresponding to the amount of the test analyte in the FFPE sample at time T₂, wherein the second IHC signal varies depending on at least the concentration of the test analyte; and applying the calibration curve to the first IHC signal and the second IHC signal of the test antigen in the FFPE sample to determine the amount of the test antigen in the FFPE sample.

The method of the present invention includes generating an IHC signal corresponding to amount of the QIRS test FFPE sample at the test time, T2 after the collection time and the amount of the test analyte in the test FFPE sample at the test time. IHC as used herein may be generally defined as the demonstration of a cell or tissue constituent in situ by detecting specific antibody/aptamer-antigen interactions where the antibody/aptamer has been tagged with a visible label. The visual marker may be a fluorescent dye, colloidal metal, hapten, radioactive marker, or more commonly an enzyme. Experimental samples include FFPE samples. Ideally, maximal signal strength along with minimal background or non-specific staining are required to give optimal antigen demonstration. IHC protocols are well known in the art; see, e.g., Immunocytochemical Methods and Protocols (second edition), edited by Lorette C. Javois, from Methods in Molecular Medicine, volume 115, Humana Press, 1999 (ISBN 0-89603-570-0).

The IHC signal for either the QIRS or test analyte in the FFPE cell or tissue sample may be generated according to known methods. To determine the amount of an antigen in a cell or tissue sample, an antibody itself, a secondary antibody that binds to the first antibody, or an aptamer can be detectably labeled. Alternatively, the antibody or aptamer can be conjugated with biotin, and detectably labeled avidin (a polypeptide that binds to biotin) can be used to detect the presence of the biotinylated antibody or aptamer. Combinations of these approaches (including “multi-layer sandwich” assays) familiar to those in the art can be used to enhance the sensitivity of the methodologies. Some of these protein-measuring assays (e.g., ELISA or Western blot) can be applied to lysates of test cells or tissues, and others (e.g., immunohistological methods or fluorescence flow cytometry) applied to unlysed tissues or cell suspensions. Methods of measuring the amount of a label depend on the nature of the label and are known in the art. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or □-glactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). Other applicable assays include quantitative immunoprecipitation or complement fixation assays.

In a preferred embodiment, the QIRS and test antigens are examined by simultaneous IHC dual or double stains. These “dual” or “double” stains including a first ‘stain’ for a Quantifiable Internal Reference Standard, and a second ‘stain’ for the unknown ‘test’ analyte. The amount present of the unknown ‘test’ analyte (protein) may then be measured with accuracy (degree thereof to be established) by comparison of the intensity of stain of the ‘test’ analyte with the intensity of stain of the internal reference standard, using validated quantitative IHC protocols and existing image analysis equipment and software. Having previously established the extent to which the internal reference standard(s) is preserved following FFPE with optimized AR, then a ‘correction factor’) and a ‘relative loss factor’ can be applied to provide a quantitative measurement of the amount of unknown test analyte present in the tissue prior to the initiation of sample preparation (i.e., when it was removed from the patient).

Optimal Antigen Retrieval and Exemplary Protocols

In a preferred embodiment of the present invention, the methods described herein include optimal antigen retrieval (AR) to achieve a maximal degree of retrieval that provides a comparable level of IHC staining among various FFPE tissue sections that have been fixed in formalin from 4 hours to 7 days. The use of optimized AR protocols permits optimal retrieval of specific proteins (antigens) from FFPE tissues to a defined and reproducible degree (expressed as R %), with reference to the amount of protein present in the original fresh/unfixed tissue. This may be explained mathematically as follows. Suppose the amount of a protein in a fresh cell/tissue=Pf, and that Pf produces an IHC signal in fresh tissue of ∫ (Pf). In FFPE fixed tissue the signal may be less due to antigen ‘loss’. When the IHC signal of FFPE is ∫ (Pffpe), then the retrieved rate of AR (R %) is calculated as: AR rate (R %)=∫(Pffpe)/∫ (Pf)×100%, the amount of protein in the FFPE tissue of Pffpe=Pf×R %. In a situation where optimized AR is 100% effective, then Pffpe=Pf, if the IHC signal is of equal strength in fresh tissue and FFPE tissue. In this embodiment, optimized AR will be carried out for the QIRS, and the intensity of IHC staining obtained for the test analytes in the same tissue section, after optimized AR, is compared with the IHC staining of a comparable QIRS to provide a measure of the amount present of the test analyte as described herein.

The vast majority of antigen retrieval studies have been applied to formalin fixed material. When aldehyde-based fixatives are used (e.g., formalin), inter- and intra-molecular cross-links are produced with certain structural proteins, which are responsible for the masking of tissue antigens. With aldehyde based fixatives, this adverse effect has been thought to be due to the formation of methylene bridges between reactive sites on tissue proteins. These reactive sites include primary amines, amide groups, thiols, alcoholic hydroxyl groups, and cyclic aromatic rings. The degree of masking of the antigenic sites depends upon the length of time of fixation, temperature, concentration of fixative, and the availability of other nearby proteins able to undergo cross-linkages. The methods of the present invention preferably include methods to “unmask” these antigenic sites a range of antigen retrieval according to known techniques.

For example, the protein cross-links formed during formalin fixation can be partially disrupted by the use of proteolytic enzymes of which trypsin is the most widely used. Trypsinization time is extremely important and is proportional to the specimen fixation time. There is a very fine balance between over and under digestion. Trypsin is optimally active at 37° C. and at pH 7.8. The reaction rate is improved by the addition of the co-enzyme calcium chloride (0.1%). Trypsin only remains active for about 30 minutes; therefore if the incubation time exceeds this, the working solution must be replaced. Not all antigens require proteolytic digestion. Furthermore, care must be taken to avoid creating “false” antigenic sites, as some antigens may be altered or destroyed by trypsinization. In some instances immunostaining may be impaired or completely removed following trypsinization. Proteolytic digestion has largely been replaced by heat mediated antigen retrieval methods.

The rationale behind these heat pretreatment methods is unclear and several theories have been postulated. One theory is that heavy metal salts act as a protein precipitant, forming insoluble complexes with polypeptides and that protein precipitating fixatives frequently display better preservation of antigens than do cross-linking aldehyde fixatives. Another theory is that during formalin fixation inter- and intra-molecular cross methylene bridges form linkages and weak Schiff bases. These cross linkages alter the protein conformation of the antigen such that a specific antibody may not recognize it. It is postulated that heat mediated antigen retrieval removes the weaker Schiff bases but does not affect the methylene bridges so that the resulting protein conformation is intermediate between fixed and unfixed.

Antigens masked during routine fixation and processing can be revealed by using high temperature, heat mediated antigen retrieval techniques; microwave oven irradiation, combined microwave oven irradiation and proteolytic enzyme digestion, pressure cooker heating, autoclave heating, water bath heating, Steamer heating, or high temperature incubator.

One Exemplary IHC Protocol is as Follows:

I. Preparation of Sections

Prepare Slides According to A. or B.

A. Deparaffinization

1. Label all slides clearly with a pencil, noting antibody and dilution.

2. Deparaffinize and rehydrate as follows: three times for 5 minutes in xylene; two times for 5 minutes in 100% ethanol; two times for 5 minutes in 95% ethanol; and once for 5 minutes in 80% ethanol.

3. Place all sections in endogenous blocking solution (methanol+2% hydrogen peroxide) for 20 minutes at room temperature.

4. Rinse sections twice for 5 minutes each in deionized water.

5. Rinse sections twice for 5 minutes in phosphate buffered saline (PBS), pH 7.4.

B. Deparaffinization and High Energy Microwave Antigen Retrieval

1. Label all slides clearly with a pencil, noting antibody and dilution.

2. Deparaffinize and rehydrate as follows: three times for 5 minutes in xylene; two times for 5 minutes in 100% ethanol; two times for 5 minutes in 95% ethanol; and once for 5 minutes in 80% ethanol.

3. Place sections in a Coplin jar with dilute antigen retrieval solution of choice (e.g., 10 mM citric acid, pH 6). Completely cover the slide.

4. Place Coplin jar containing slides in vessel filled with water and microwave on high for 2-3 minutes (700 watt oven).

5. Check level of retrieval solution, allow to cool for 2-3 minutes, and repeat steps 3 and 4 four times (depending on tissue). Completely cover the slide.

6. Remove Coplin jar containing sections and allow to cool for 20 minutes at room temperature.

7. Rinse sections in deionized water, two times for 5 minutes.

8. Place slides in modified endogenous oxidation blocking solution (PBS+2% hydrogen peroxide).

9. Rinse slides once for 5 minutes in PBS.

II. Blocking and Staining

1. Block all sections with PBS/1% bovine serum albumin (PBA) for 1 hour at room temperature.

2. Incubate sections in normal serum diluted in PBA (2%) for 30 minutes at room temperature to reduce non-specific binding of antibody. Perform the incubation in a sealed humidity chamber to prevent air-drying of the tissue sections.

3. Gently shake off excess antibody and cover sections with primary antibody diluted in PBA. Replace the lid of the humidity chamber and incubate either at room temperature for 1 hour or overnight at 4° C.

4. Rinse sections twice for 5 minutes in PBS, shaking gently.

5. Gently remove excess PBS and cover sections with diluted biotinylated secondary antibody in PBA for 30 minutes-1 hour at room temperature in the humidity chamber.

6. Rinse sections twice for 5 minutes in PBS, shaking gently.

7. Remove excess PBS and incubate for 1 hour at room temperature in Vectastain ABC reagent (as per kit instructions). Secure lid to humidity chamber to ensure a moist environment.

8. Rinse twice for 5 minutes in PBS, shaking gently.

III. Development and Counterstaining

1. Incubate sections for approximately 2 minutes in peroxidase substrate solution made up immediately prior to use as follows:

10 mg diaminobenzidine (DAB) dissolved in 10 ml 50 mM sodium phosphate buffer, pH 7.4;

12.5 μl 3% CoCl₂/NiCl₂ in deionized water; and

1.25 μl hydrogen peroxide.

2. Rinse slides well three times for 10 minutes in deionized water.

3. Counterstain with 0.01% Light Green acidified with 0.01% acetic acid for 1-2 minutes depending on intensity of counterstain desired.

4. Rinse slides three times for 5 minutes with deionized water.

5. Dehydrate two times for 2 minutes in 95% ethanol; two times for 2 minutes in 100% ethanol; and two times for 2 minutes in xylene.

6. Mount slides.

As have been described above, the methods of the present invention include at least some of the following characteristics:

1. candidate QIRS molecules, antigens such as proteins, are selected on the basis of their widespread presence in recognizable cells in all (or almost all) tissues;

2. The exact amount of protein (QIRS) present on a per cell basis (averaged across 100 or 1000 cells) is measured experimentally in fresh tissue, by independent techniques, such as ELISA (enzyme linked immunosorbent assay) assay of extracts containing known numbers of the critical cell type (that contains the protein). This protein, once validated, constitutes a QIRS for a test analyte. Controlled IHC is performed on the fresh tissue and the intensity of IHC QIRS signal per cell is recorded (by computer assisted quantified image analysis) in relation to the measured amount of protein present, determined as above by independent methods.

3. The quantitative amount of the QIRS in the same cell type is then determined experimentally (by the same methods) following sample preparation (FFPE). Controlled IHC is performed on the FFPE tissue and the intensity of THC signal per cell is recorded (by computer assisted quantified image analysis) in relation to the measured amount of protein present.

4. Comparison of the IHC signal of the QIRS for the FFPE tissue with that of the fresh tissue then allows calculation of the loss of signal intensity attributed to loss of the reference protein during FFPE. This loss can be expressed as a percentage or as a ‘coefficient’ of loss due to fixation.

5. Selected proteins of interest (test analytes) that are variably present in pathologic tissues, and that require a quantitative analysis for therapeutic decisions (diagnosis or prognosis), are then subjected to the identical process under controlled conditions. The loss during sample preparation for each selected test analyte (coefficient of loss due to fixation) is then derived experimentally, and the data recorded.

6. Having established a system of QIRS as described, it is then possible to take a surgical biopsy and determine by weight the amount of test analyte of interest present on a cell to cell basis by employing double IHC staining using the QIRS as the calibrator with comparative spectral imaging (computer assisted image analysis) of the signal for the test analyte.

The following examples are intended to illustrate, but not to limit, the scope of the invention. While such examples are typical of those that might be used, other procedures known to those skilled in the art may alternatively be utilized. Indeed, those of ordinary skill in the art can readily envision and produce further embodiments, based on the teachings herein, without undue experimentation. All publications cited herein are incorporated by reference in their entirety.

### EXAMPLES

### Example I

### Quantification of Immunohistochemistry—Issues Concerning Methods, Utility and Semi-Quantitative Assessment

### SUMMARY

Immunohistochemistry now is entering its fourth decade of use on formalin fixed paraffin embedded tissues. Over this period the method has evolved to become a major part of the practice of diagnostic surgical pathology worldwide. From the beginning immunohistochemistry has been adapted to provide a range of markers of cell lineage and tissue type, with particular application to the diagnosis and classification of tumors. In this modality immunohistochemical methods were employed simply as ‘special stains’, the results of which were evaluated quantitatively by the pathologist, as for any other stain. More recently, attention has shifted to the demonstration of prognostic markers in tumor cells, driven by the advent of molecular biology and the discovery of numerous regulatory molecules, coupled with manufacture of the corresponding specific antibodies. Immunohistochemistry has rapidly adapted to this new use, but in so doing the demand for some form of quantification has become paramount; it is no longer enough that the ‘stain’ is there; rather it is a question of “How much is there?” This review explores the limitations of immunohistochemistry when employed in a semi-quantitative mode, and explores the possibility of fulfilling the full potential of immunohistochemistry, as a true quantitative immunoassay applied in a tissue section environment.

### DEFINITIONS

Quantity (noun): 1 a certain amount or number, 2 the property of something that is measurable in number, amount, size or weight, 3 a considerable number or amount (from Latin, quantitas—how much?).

Quantitative (alt. quantitive) (adjective): of, concerned with, or measured by, quantity. (Oxford Dictionary Compact Edition, Oxford University Press, 2002).

The term “semi-quantitative” lacks clear definition, but would imply having some of the features of “quantitative”, as in “semi-precious”, or not quite precious, and relying upon subjective judgement.

While these definitions have some clarity in certain contexts, the use of the term “quantitative” in Anatomic Pathology is uncommon and inconsistent. By way of contrast, within the Clinical Laboratory many assays are quantitative, and the characteristics that make up a quantitative assay can there be examined at leisure.

Anatomic pathology (surgical pathology, histopathology) per se is primarily observational, dependent upon pattern recognition in its broadest sense, without overt acknowledgement that within the context of pattern recognition there are elements that are quantitative. Biological stains, introduced in the mid-19^{th} century [review, Conn's Biological Stains (1)], lend tinctorial properties to the tissue section. The interpretation of even the simple routine H&E stain does include elements of a quantitative assessment, albeit mostly at a subconscious level. Are the nuclei more or less blue (hyperchromatic)? Is the cytoplasm of the cardiac myocytes pinker than normal (hypereosinophilic), as in the early phases of myocardial infarction? What amount of atypia is present? These evaluations are made subjectively, with experience as the reference point, and formal quantitative methods are not usually employed, except for particular defined purposes (2). Assessment of the degree of malignancy, formalized in some instances into grading criteria, again includes quantitative elements, such as the number of mitotic figures (sometimes going so far as to offer a count per high-power field), or the number of large cells versus small cells in a population, as in the grading of diagnosed follicular center cell lymphomas of B cell origin. Underlying these “semi-quantitative” approaches there is the subliminal concept of a covert reference standard, against which judgments, rather than “measurements”, can be made. Often this standard is crude as in the use of a “normal histiocyte” nucleus to separate large from small in the grading of FCC lymphomas, and the level of diagnostic agreement amongst different observers, including experts, is disturbingly poor [about 60% in this instance—The Non-Hodgkin's-Lymphoma Classification Project (3)].

Faced with the limited application of quantitative methods in day-to-day surgical pathology, a comparison with the quantitative methods in use in Clinical Pathology is of real value in determining how to improve the situation. Biological stains (including those based on aniline dyes) that are the basis of the usual histopathologic stains are somewhat difficult to control in terms of intensity of color (stain), from cell to cell and more so from section to section (different tissues on different days), although this may change with the advent of new generations of automated stainers. An immunohistochemical (IHC) reagent, by contrast, has the potential to provide quantitative data, for although we are not accustomed to thinking of it as such, it is in potential, if not in fact, an “immunoassay” performed in situ on the tissue section. An IHC “stain” is strictly analogous to an ELISA (enzyme-linked immunosorbent assay) test performed in the clinical lab, and ELISA tests are widely recognized as being truly quantitative (if properly performed). Exactly the same reagents that are employed in an ELISA test on serum, for example, an assay for insulin, may be employed to perform an IHC stain for insulin in a paraffin section. It is a curious oversight of scientists in general, and pathologists in particular, that the principles and reagents used in one environment are accepted as providing a strictly quantitative result (ELISA-serum), but when applied to a tissue section (IHC), are addressed only as a “stain”.

Factors to be Addressed in Establishing Quantitative IHC Methods; Towards an IHC Assay as Opposed to an IHC Stain

There have been several schools of thought as to the reason why IHC “stains” are difficult to run in a manner that lends itself to quantitative analysis. If there is a consensus, it is that several reasons conspire together; these may conveniently be grouped into three general areas (Table 1).

TABLE 1
The Total Test, adapted from the earlier proposal of the US Biologic
Stain Commission (4), and modified from “Immunomicroscopy: A
Diagnostic Tool for the Surgical Pathologist,” Taylor CR and Cote RJ (5).
The Total Test

Pre-analytical:
Specimen handling, from operating room to histology lab
Fixation: total fixation time, and type of fixative
Paraffin embedding, storage and sectioning
De-paraffinization
Analytical:
Antigen retrieval (exact method)
Assay (staining) method and protocol
Reagent validation
Controls (Reference Standards)
Technologist and laboratory certification
Proficiency testing and quality assurance
Post-analytical:
Reading of result(s)/scoring/quantification
Report
Turn-around time
Outcomes analysis/economics/reimbursement

Possibly the overriding factor in effecting significant change would be to transform the mindset of pathologists, at least of the next generation, such that the end-result of an IHC protocol would come to be regarded NOT as just a stain, but rather as a precise immunoassay that is strictly quantifiable, if properly performed and controlled, similar to any other immunologically based assay of like principle (such as ELISA).

It would seem evident that in order to achieve a quantifiable result with an IHC stain, thereby converting it to a quantifiable immunoassay, the total assay (staining process) must itself first be standardized (6-10). Those areas in assay performance that lead to significant variability or errors, and are therefore targets for improvement, are reviewed below.

### Pre-Analytic Issues: Transportation, Fixation, Sectioning

Pre-analytical issues fall under the broad rubric of “sample preparation” (Table 1). This area is the least well controlled of all phases of the IHC staining process (6,11), and the least controllable, because of the ways in which tissues are obtained from diverse hospital and clinic settings. At long last the importance of good sample preparation in cancer diagnosis, or misdiagnosis, particularly with regard to measurement of prognostic and predictive markers, has reached the national consciousness in the United States, with issuance of requests for proposals from the NCI (RFA-CA-07-003: Innovations in Cancer Sample Preparation, U.S. National Cancer Institute, 2006).

In the ‘routine’ environment of diagnostic surgical pathology, specimens that ultimately may be subject to IHC analysis may be obtained by fine-needle aspiration, or from the operating room by biopsy, or by more extensive therapeutic surgical procedures. Following removal of the tissue from the body, autolysis generally is arrested by immersion in a fixative. By far the most commonly employed fixative is formalin (in common practice a 4% solution of formaldehyde) (6,11,12). Other fixatives have been employed, and others are being explored, in order more effectively to meet some of the current needs for performing molecular analyses of tissues or cells (13). Formalin has many advantages, not least a long tradition of use and the fact that it yields good morphologic detail; or rather it yields the morphologic detail we are accustomed to, which is deemed the equivalent of good. Formalin also is inexpensive, easily stored (with some reservations as to quality), and universally available. Formalin, therefore, is what we have, and what we must learn to work with for the immediate future.

Recognizing that the autolytic process begins immediately, the routine practice is to place the excised tissue directly in formalin, prior to leisurely transportation the laboratory, with emphasis on leisurely. Once in the surgical pathology suite (“grossing” room) the specimen is cut in, meaning that if not already sufficiently small it is cut into small blocks to facilitate rapid penetration by the fixative (formalin penetrates relatively slowly), and placed in fresh fixative for further processing. Whereas the ideal time for fixation of a 5-mm-thick tissue block is perhaps 12-24 hours [no uniform agreement here (11,12)], in practice, the total time in fixative is very variable, due to differing transportation times to the laboratory and accumulation of specimens for batch processing. Fixation time in reality is almost entirely uncontrolled, varying anywhere from 6-24 hours, or more. Add to this, questions as to whether the formalin is freshly prepared and adequately buffered, plus variability in the rate of penetration of formalin in different types of tissues and into differently sized blocks, and the result is a major impediment to standardization of an IHC stain, and an obstacle to quantification.

As an aside, in-situ-hybridization (ISH) methods have a probe-target pairing that is not immunologically based, and thus strictly do not fall under the title of IHC. Nonetheless, the principles are closely analogous, particularly with reference to interpretation and scoring. For RNA analysis by ISH methods, there is a further complication, namely the rapid degradation of RNA by intrinsic enzymes, probably beginning as soon as the blood supply to the tissue is interrupted as part of excision. For useful results, and certainly for quantification, it is essential, therefore, to process such materials immediately, and control over transportation time becomes critical so as to minimize the time elapsed prior to complete fixation.

Following fixation, the process of embedding in paraffin, and subsequent de-paraffinization of the cut sections, also involves exposing the tissues (and therefore the analytes) to a series of chemicals and to heat. The end-result is a formalin-fixed paraffin embedded (FFPE) tissue section. While anecdotes exist, there are no good data as to the adverse effects of processing upon the various analytes that might be detected by IHC staining. This aspect, therefore, is usually ignored, but in the absence of data it appears sensible that these steps of the overall preparation of the tissue section are performed as consistently as possible.

The importance of consistent sectioning may also be overlooked. For routine staining a precision microtome is used to achieve a section thickness of about 5 μm. A nucleus that is 5 μm in diameter may thus be entirely within the plane of the section, or only partially included, with effects upon the apparent intensity of a nuclear IHC stain, all other things being equal. Thicker sections may manifest the same problem even for quite large nuclei, whereas generally thinner sections will minimize it. Uniform preparation of FFPE sections that are less than 5 μm in thickness is not possible; plastic embedding media, or other special media, allow consistency in sectioning clown to 1 μm, but do not lend themselves well to routine use, or to larger blocks. Even slight variations in thickness, over a 5 μM section, due to “chatter” or unevenness of cut, may also produce changes in intensity of the staining reaction that are inapparent to the naked eye, but are readily appreciable using quantitative imaging techniques. (6).

### Analytical Issues: Antigen Retrieval, Protocols, Reagents, Controls

Antigen retrieval, considered here as part of the analytic process, has shown spectacular benefits in terms of the ability of all and sundry to achieve a positively stained FFPE section, but there have been some unexpected and unwanted consequences (5,14,15,16). The fact that many antigens, that hitherto could be stained only with difficulty, now are readily demonstrable following AR has led to renewed laxity with regard to fixation, and to diminished efforts in developing alternative and superior fixatives. The AR method itself is also open to great variation in practical performance, and this may affect the intensity of stain achieved, or even the number of cells that are perceived as demonstrating a positive staining reaction. Also the degree to which any particular antigen is “retrieved” is entirely unknown with reference to the absolute amount present post-fixation (in the FFPE section), and the amount present post-fixation is itself not known with reference to the amount present (per cell) when the tissue was first removed from the body (fresh, prior to sample preparation—“pre-fixation”). Some standardization may be achieved through the practice of testing the different variables in the retrieval process (method of heating, temperature, time, pH, etc.) to achieve the optimal AR protocol for each specific antigen using a defined set of reagents and staining methods (5,15). This approach would seem to risk the possibility of uncovering significantly different AR protocols for many antigens, but in practice yields only three major variations of the basic AR method, one of which will generate excellent results for the great majority of clinically relevant antigens (5,15).

Reagents and staining protocols, once seen as the primary impediment to qualitatively reproducible staining, are now regarded as perhaps the least of the difficulties, providing that certain procedures are followed, a tribute to the fine efforts of the Biological Stain Commission/FDA working groups more than a decade ago (4,17). A common error is to neglect to read the package insert for each new reagent carefully; at a minimum, perusal will provide performance characteristics (does it work on FFPE sections?) and expected patterns of staining. It should also provide a detailed staining protocol, with a judicious reminder that should a laboratory choose to depart from the protocol, then validation becomes the entire responsibility of the performing laboratory. In any event, every new reagent introduced into the laboratory, whether a primary antibody, or a different labeled antibody system, must undergo an initial validation by the laboratory to establish the performance characteristics. So called positive-control tissues serve this purpose, and properly should have been fixed and processed in a manner identical to the test specimens (same fixative, fixation time, etc.) (4, 5, 8, 18, 19). Tissue microarrays are useful in evaluating a new primary antibody, allowing a quick and efficient study of the pattern of staining on potentially hundreds of tumor or tissue types, in duplicate or triplicate, deposited on a single slide. These basic control sections serve to validate qualitatively the reagents and protocol, but as usually constituted cannot serve as absolute reference materials for calibration and quantification. This limitation is because the control materials themselves, while demonstrably positive in a qualitative sense, have been fixed and processed in ways that preclude knowing, in absolute terms, how much of the test analyte is present post-fixation; it is merely that there is enough to detect a positive staining reaction with the reagents and protocol employed. Indeed the amount of analyte present pre-fixation (when fresh) also is totally unknown.

From this brief review it is argued that the ‘total test’ must be standardized in order for any conceivable quantitative scoring method to achieve a useful degree of reliability, and that standardization must include assessment of any deletoorious or inconsistent effects of specimen preparation, including tissue ischemia as well as fixation and processing in the laboratory. Even so, for all the reasons described, the best that can be achieved today is a ‘semi-quantitative’ type of assay, absent availability of a defined reference standard.

It follows that a primary requirement should be to develop reference materials that can be used to establish the integrity of the sample, as well as to standardize the assay and to calibrate the results. The criteria for such a standard can be derived once more by extrapolation from Clinical Pathology (Table 2).

TABLE 2
Summary of desirable characteristics of any reference standard that
would provide a basis for accurate quantification of IHC (or ISH) (19).
Immunohistochemical Reference Standard: Requirements

It must be subjected to the same rigors of sample preparation as the
“test” tissue; to include any effects of tissue ischemia, fixation and
processing
It must be integrated into all phases of the test (assay) protocol,
including evaluation of the result.
It should contain a known amount of the analyte(s) subject to assay
It should be universally available
It should be inexhaustible and inexpensive

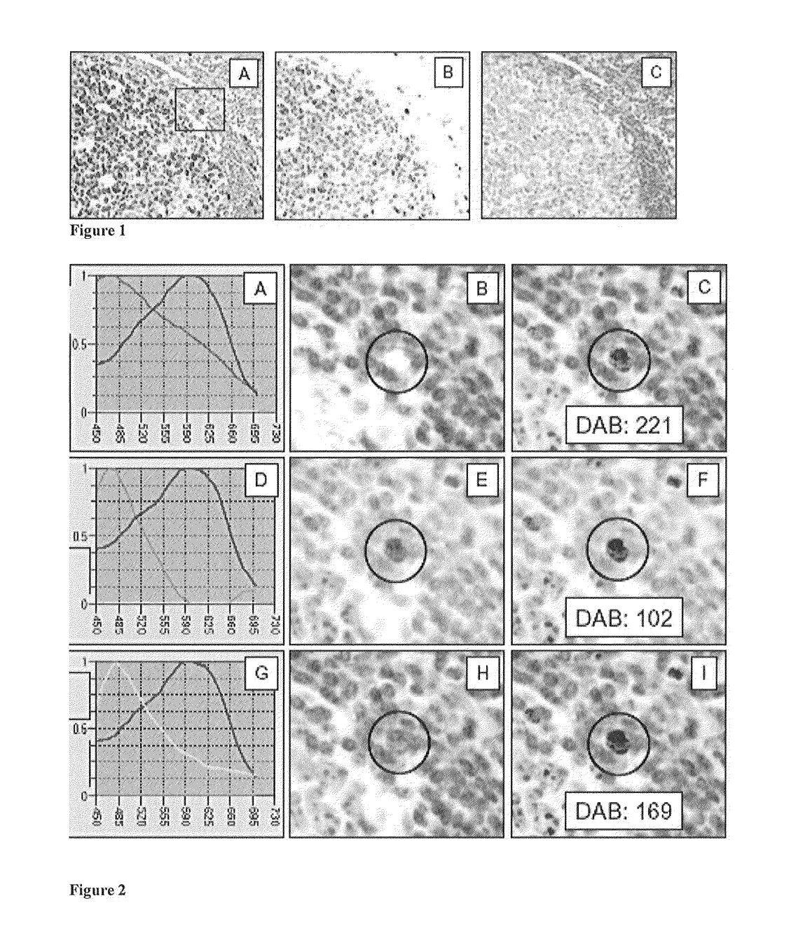

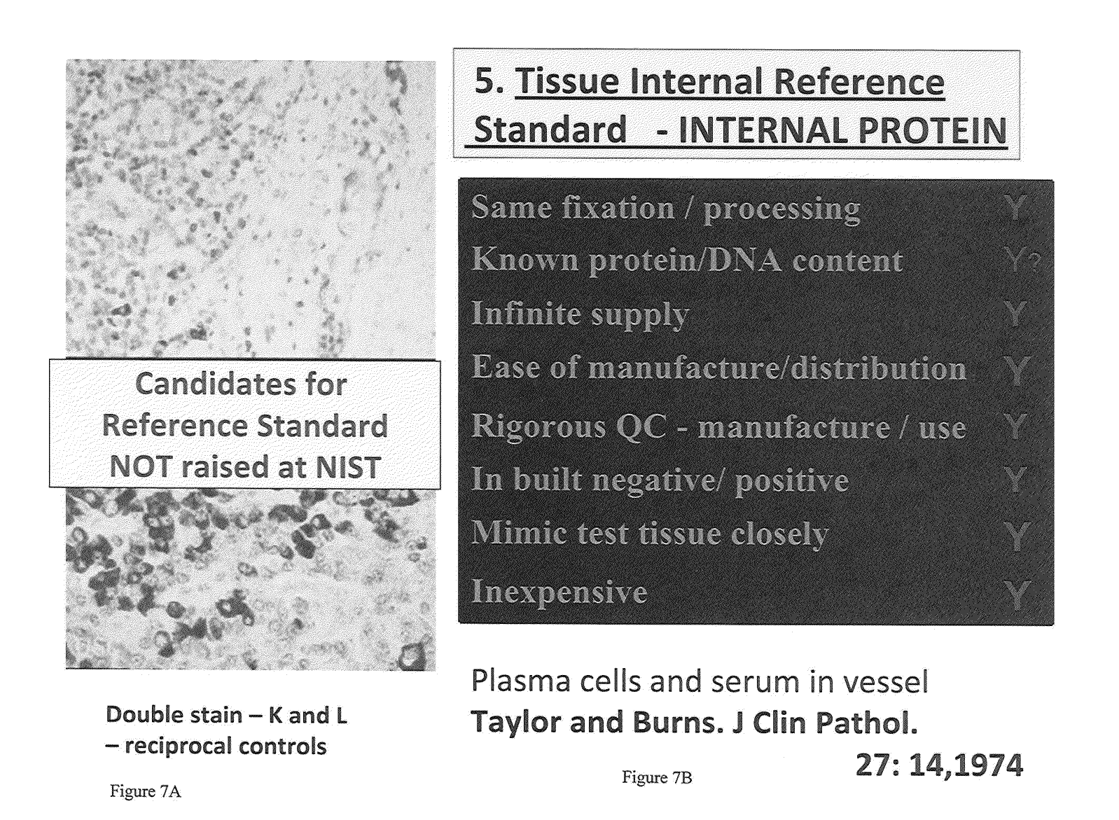

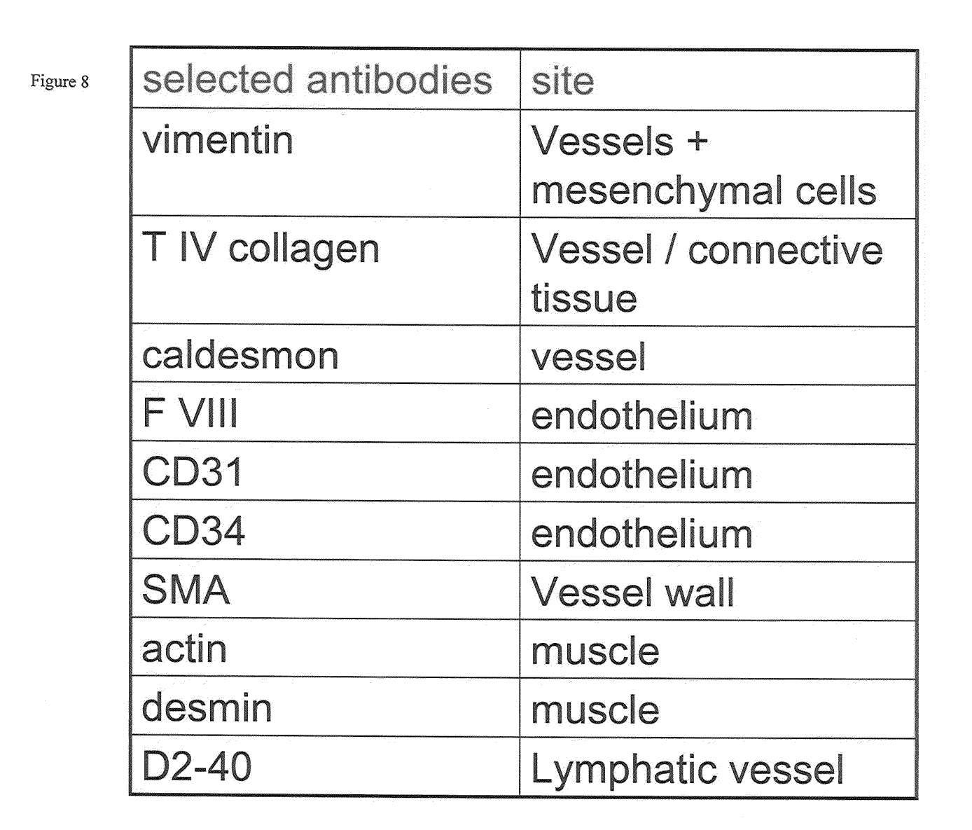

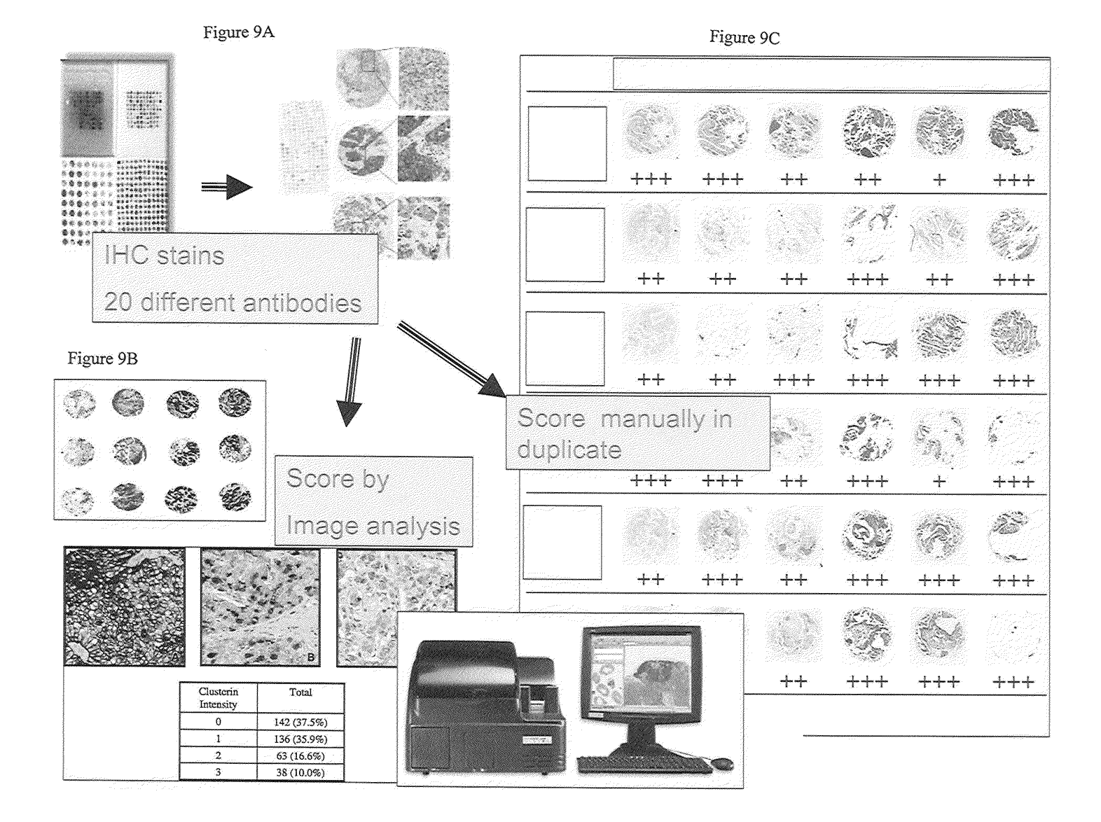

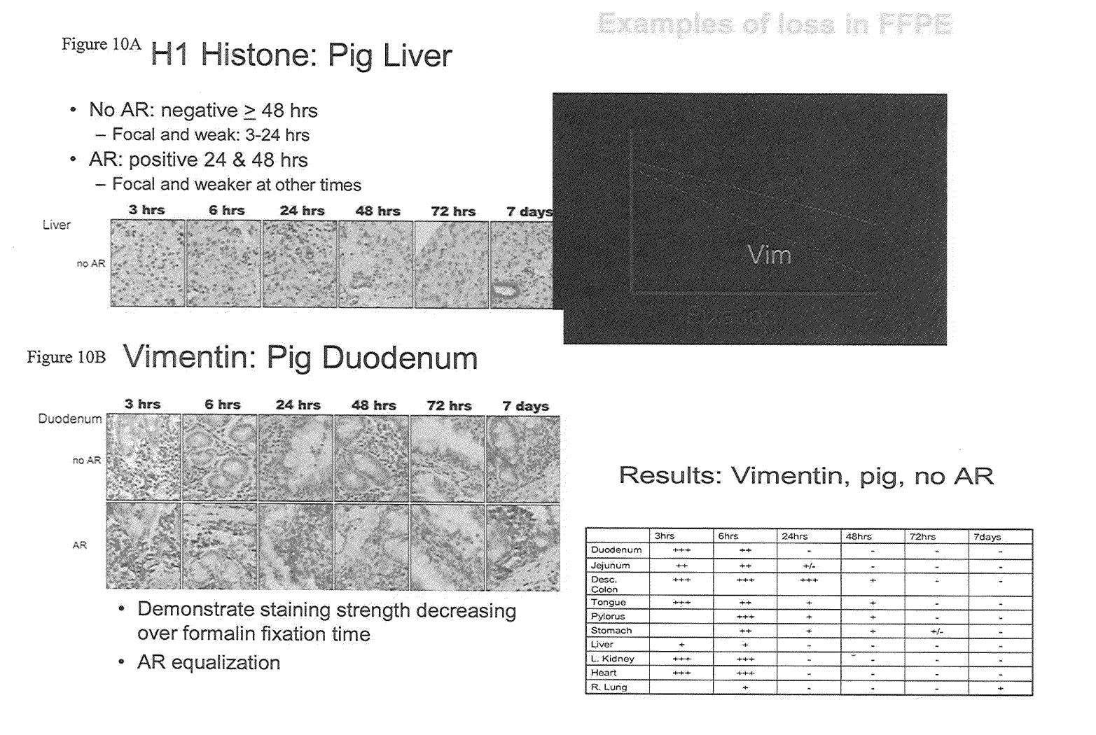

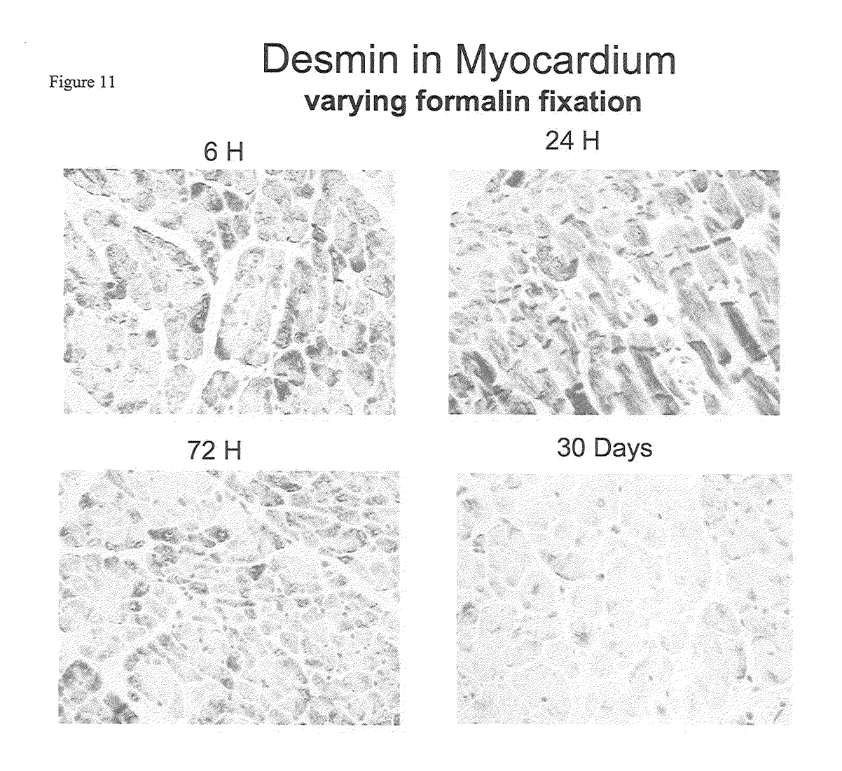

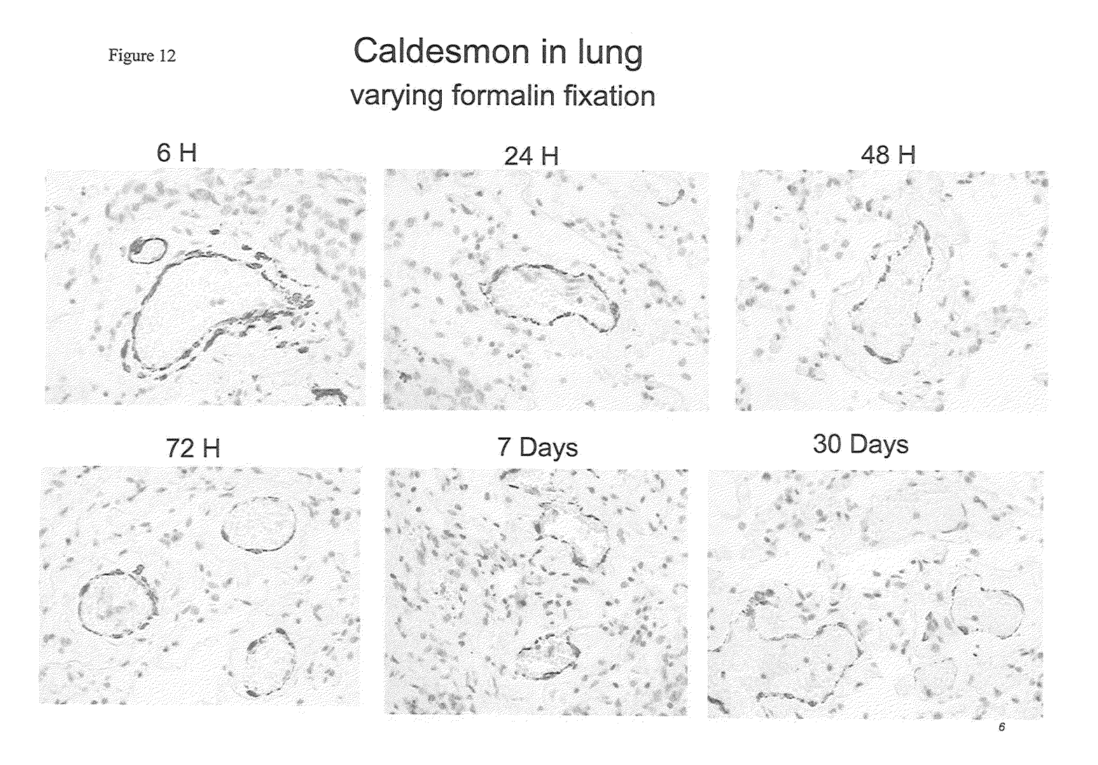

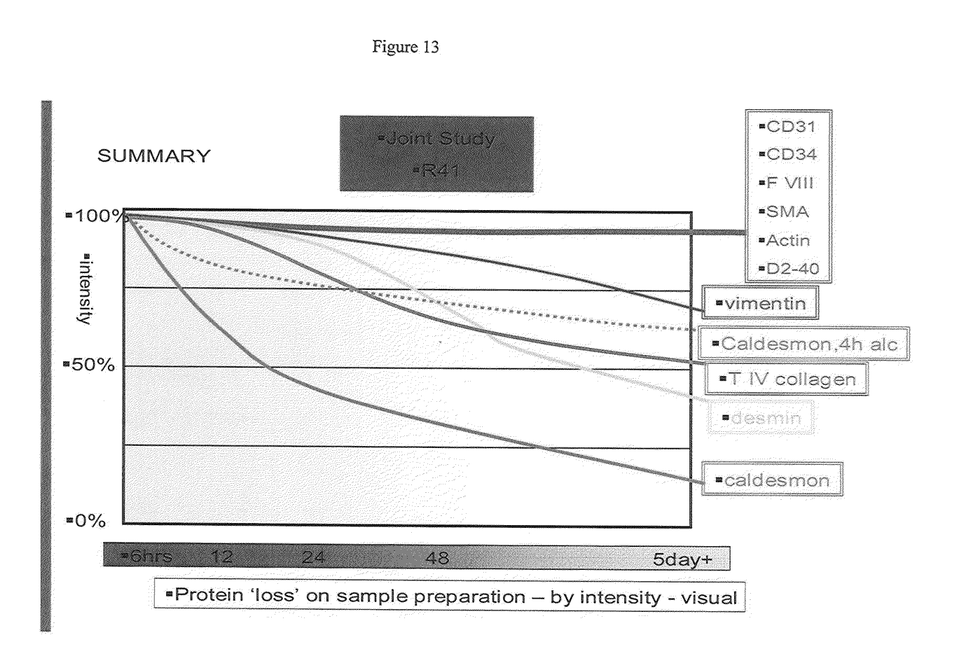

For IHC these requirements are exacting, and have yet to be fully met in a practical sense. As discussed above, the usual positive-control tissue employed in laboratories meets only some of these requirements, as does the FDA-approved Her2-kit produced by Dako (HercepTest, Dako, Glostrup, Denmark, or www.dakousa.com), which includes a reference cell line, that clearly has had different processing from the tissue being tested. In all instances the most important deficiency is the lack of data relating to the absolute amount of the analyte present in the control material prior to the first step of the total test (i.e., prior to specimen preparation/fixation, or even up to the point of its removal from the body of the patient). Efforts to meet the requirements set forth in Table 2 have been few, but do show some promise in the use either of peptide deposits (20,21), cell lines (including cell-line blocks) (22,23), or faux tissues (histoids) [Marylou Ingram and Ashraf Imam, unpublished collaboration, 2005; see reference (5), p. 35, FIG. 1-27]. (NOW PUBLISHD REF SENT BY E MAIL) One aspect of the invention is use Quantifiable Internal Reference Standards, the characteristics of which will be measured by experimental observation under differing conditions of formalin-fixation, paraffin-embedment and antigen-retrieval (19). Such internal standards, once established in terms of absolute quantity of analyte per specific cell type, have the potential to serve as calibration points for test analytes demonstrated in adjacent cells by double-IHC stain methods, using multiplex-capable imaging techniques that are described later.

Lacking quantifiable internal reference standards for calibration, all IHC stains at best can only be semi-quantitative, comparing the intensity of stain, or the number of positive cells, or both, with the control, or with other cases, with results that are relative, not absolute.

### Post Analytic: Results and Interpretation (Scoring)

One school of thought held that the lack of reliability of IHC methods for measurement of estrogen- or progesterone-receptor expression was attributable to the nature of the “semi-quantitative” scoring process, and the intrinsic deficiencies of an observer-based, subjective manual method. The underlying belief was that, however clearly the criteria are set forth, the application of such criteria and the reporting of the outcome will vary from pathologist to pathologist, or even for the same pathologist from day to day. Computer assisted image analysis was a touted solution to the scoring of IHC stains, where a quantifiable result was the desired outcome. Comparative studies (7,9,24,25) indeed do show that under controlled circumstances image analysis is superior to manual methods as performed by most observers.

The problem of interpretation of an IHC stain should not be minimized. With basic lineage-related markers, the problem of consistent evaluation is real, even with reference to relatively simple questions: is the cell or tissue positive for kappa chain or ER or CD30, or is it not? Is specific staining present or not, with reference to the controls? Where is the staining localized? How much staining is there (begging the questions as to whether the amount of staining correlates with the absolute amount of antigen)? What scoring system should be used and how reproducible is it? The general consensus is that IHC methods, applied as qualitative ‘special’ stains, if properly applied and interpreted, increase the accuracy of diagnosis in surgical pathology, as is well established by studies of lymphoma (3). However, it is known, though not often publicly acknowledged, that the eyes and brains of different observers do not see and interpret the same H&E section the same way (18,26,27). For IHC stains the variability of interpretation may be even greater, as is revealed in some of the proficiency-testing exercises carried out by the CAP (College of American Pathologists, Chicago, USA) and UK NEQAS-ICC (United Kingdom, National External Quality Assessment Scheme Immunocytochemistry). It turns out that the answers are dependent not only upon the experience and acuity of the eye of the beholder, but also upon the integrity of the staining process as already emphasized (6,7,9,10,28,29,30).

With respect to prognostic markers the problem of inter-observer consistency is much greater, requiring not just a decision as to whether there is specific positive staining, or not, but some sort of scored or semi-quantitative result. The inherent difficulties are well recognized for such commonly tested analytes as ER and PR (28), where commercially available reference standards are not usually available, and where both methodology and scoring vagaries contribute to error. The problem is arguably even greater for Her 2 (29, 30). The FDA-approved Dako kit contains a cell-line standard and includes instruction about how to read the result, and most published reports utilize some form of reference control. Even with these important provisions, scoring of the same cases for Her2, ER and PR by residents and pathologists shows clinically important variations and is short of the desired uniformity (28,29,30).

Some investigators believe that the solution to the problem of interpretation, especially the quantitative or scoring aspects of interpretation, may be found in improved methods of image analysis (7,9,24,25). Methods and instruments currently exist that yield improved results; many of these instruments are available commercially. At present, the larger reference laboratories are more likely to use such aids than smaller laboratories, or even academic centers. In part this is a matter of economics; the instruments are expensive and hard to justify where volumes are insufficient, or where special expertise cannot be developed and committed to their operation. In part it is a reflection of the fact that image analysis still requires interactive input by the pathologist, and that often leads to increased time requirements for reading the assay without conclusive evidence that the result is of more value clinically. Nonetheless, a visit to the exhibitor display at any of the major pathology meetings leaves little doubt as to which way the wind is blowing, as reviewed in the following paragraphs.

The last decade has seen enormous advances in the capabilities of image analysis systems applied to tissue sections, both in software and hardware, especially in digital cameras and in data management of the resulting large files. However, realization of the potential for increased accuracy in the post-analytic phase of the assay has served to focus renewed attention on the basic deficiencies of the IHC staining process as a whole, and its intrinsic lack of reproducibility, as discussed in the first part of this article. Even the most sophisticated image analysis hardware/software system cannot produce accurate results if the underlying stain (read immunoassay) itself suffers from non-reproducibility or significant non-linear behavior. In this context accuracy (and reproducibility) can only be determined if rigorous quantifiable reference standards (19) are available and are used to calibrate the system. The notion of accuracy should embrace not only the measurement of an analyte in a particular section, validated against a reference standard, but also the ability to repeat the result on the same case, day to day, in the same and in different laboratories, and the ability to measure the same (and ultimately different) analyte(s) in different specimens and cases, again reproducibly. Thus standardization of the overall assay must proceed hand-in-hand with accurate and reliable reading (scoring) of the assay; both are essential for achievement of an IHC stain, which in practice could be, and should be, more than just a stain but rather a system of controlled and interlocked processes, analogous to immunoassays in the clinical laboratory.

Finally, expression-array-based research has emphasized that pathology and in particular, cancer biology, reflects the simultaneous workings of multiple molecular pathways. For maximum relevance, these should be assessed on a per-cell, rather than a per-tissue-slice basis, since ultimately cells are the units of behavior, and their individual phenotypes are the relevant metric. In a practical sense this implies multiplexed molecular (IHC or ISH) assays in which more than one analyte is assessed on a tissue section at one time, in identifiable individual cells. As can be imagined, in addition to the imaging challenges this may pose, it also amplifies all the demands on controls and standards elaborated above.

### Image Analysis; Approaches and Systems

While image analysis of molecular labels can include a number of applications, the following section will be limited to the discussion of the problem of estimating abundance of stains in histological tissue, with an emphasis on IHC as opposed to immunofluorescence. The previous section has addressed issues of sample preparation and provision of appropriate controls that can ensure that the IHC procedures have generated a valid signal for the imaging system to capture. The assumption is made that the signal on the slide is representative and in some way quantitatively related to the abundance of the antigens in the tissue section, which in turn is related, albeit in ways unknown, to the absolute amount of the analyte in the original tissue. The example used herein will be estimation of nuclear antigens rather than membrane-staining, since the latter may require additional considerations beyond simple intensity measurements, such as spatial patterns of expression that have their own subtleties. In addition this review will not dwell on the well-documented subjectivity and intra- and inter-observer variability of manual, visual-based semi-quantitative estimation of intensity or even of per-cent-positivity (31,32), and will simply postulate that properly designed automated imaging methods, because they are immune to the consequences of fatigue and subjectivity, can outperform human observers, certainly in terms of precision and quantitative reproducibility.

Factors that affect performance of the imaging system include the choice of camera and illumination source, the optical performance of the stains themselves, as well as the presence and degree of multiplexing. After image acquisition, it is then necessary to deploy appropriate mathematical techniques to extract quantitative intensity and area measurements from the imaging data.

Imaging Hardware: RGB Vs. Multispectral Approaches

There is a long history of the application of image processing to pathology samples (33). While some early automated imaging systems employed grayscale cameras and filter wheels to collect images, most current brightfield (transmitted light) pathology imaging systems rely on standard color cameras similar in many respects to consumer digital cameras. These typically employ a Bayer-pattern color mask over a CCD or CMOS detector, and use various algorithms to process the raw image data to generate color images that can be presented to the pathologist, and that are also used in downstream automated analysis. Single-chip, Bayer-pattern red-green-blue (RGB) cameras that are often employed, especially in many “home-grown” systems, can generate imaging artifacts, especially with respect to fine structures or edges, and have poorer spatial fidelity than more expensive 3-chip systems in which separate pixel-registered cameras are used to acquire simultaneously red, green and blue images. While the simple acquisition of good-looking color images is appealing, RGB detectors can introduce significant problems when one is trying to achieve quantification and inter-instrument precision. There are a number of ways that variation arises. For example, color values can vary significantly with the color temperature of the illumination source, different color-correction routines in camera firmware can play a role in the exact color values that are reported out, and different camera chips have differing spectral responsiveness. Some cameras employ automatic gain control or related circuitry designed to “optimize” image quality, with unpredictable effects on resulting images.

Even if an RGB imaging system is working perfectly, there are intrinsic limitations to its ability to distinguish between similar chromogens, and even more challengingly, to be able to “unmix” such signals if they overlap spatially. “Unmix” in this sense means to isolate the optical signal from each chromogen so that each can be measured quantitatively, and separately. Signal processing theory suggests that at least n if not n+1 measurements are needed to unmix n signals. In theory, therefore, it is impossible to unmix more than 3 chromogens with an RGB sensor. In practice, while it is possible to do a good job unmixing DAB (brown) from hematoxylin (blue), it has proven extremely difficult to unmix brown from red from blue (a typical combination of stains for a double-labeled sample), using only RGB measurements, due to the color-overlap of the spectral profiles. To accomplish such tasks properly, true multispectral imaging approaches may be necessary.

### Spectral Imaging

Spectral imaging microscopy represents a technological advance over visual or RGB-camera-based analyses. By acquiring a stack of images at multiple wavelengths, spectral imaging allows the determination of precise optical spectra at every pixel location. With this spatially resolved spectral information in hand, it is possible to enhance the utility of IHC and ISH stains, and even the standard biologic stains used in surgical pathology. There are a number of ways to perform spectral imaging, reviewed in (24,35). The focus in this review is on the commercially available liquid crystal tunable filter-based system (Nuance™, CRi, Woburn, Mass.), from which all examples here will be drawn; this is not to imply that the Nuance system is the best or only approach, merely that it is the model with which the authors have had most experience. This system is suitable for both brightfield and fluorescence imaging. Under automatic control, a series of images (from 3 to as many as 20 or more) are taken from blue to the red (e.g., 420 nm to 700 nm) and the resulting image “stack” or “cube” is assembled in memory in such a way that a spectrum is associated with every pixel. The ability to sample the spectrum with many discrete wavelength regions spanning the visible wavelength range allows for accurate unmixing of multiple spatially co-localized chromogens, even if they are similar in color and have largely overlapping absorption spectra. Thus, it becomes straightforward to separate dark reds from light browns, or even varieties of blue stains (hematoxylin vs. NBT-BCIP) (36,37).

### Image Processing and Unmixing

The key process, either with RGB images or multispectral datasets, is to partition the overall signal in a given pixel correctly into its component species. Linear unmixing algorithms (as described in (38,39,40) rely on the signals adding together linearly. This is true with fluorescent dyes (which emit light), but this is not the case with chromogens imaged in brightfield, since they absorb light. Fortunately, the Lambert-Beer (or simply Beer's) law relating concentrations to absorbance indicates that when the transmission data is converted to optical density (absorbance) units, linearity is restored, and quantification and unmixing (39) can be successfully achieved. There are many benefits attendant on the conversion to optical density (OD), which is typically performed by taking the negative (base 10) log of the transmitted image divided by the illumination (usually a clear area on the microscope slide). First, absorbance values are an intrinsic property of the sample, and do not depend on vagaries of illumination or camera responsivities. This means that absorbance measurements of a given specimen performed on any appropriate system should, in theory, be comparable. Secondly, in the process of creating an absorbance image, flat-fielding is automatically performed, which removes the effects of uneven illumination and minor flaws in the optical train. Conversion to OD can be performed on monochrome, RGB or multispectral images.

OD (absorbance) units are dimensionless and logarithmic: so that zero absorbance means all photons transmitted; an OD of 1.0 absorbs 90% of all photons, and an OD of 2.0 absorbs 99% of all potentially detected photons. IHC stains can individually generate signals of 1 OD. Accordingly, having 2 or more dense and overlapping stains can result in virtually black deposits from which little or no useful spectral or quantitative data can be recovered. This, plus the lesser dynamic range achievable with IHC vs. fluorescence-based approaches may mean that immunofluorescence may be preferable or necessary for some applications (32). Nevertheless, IHC has some practical advantages over immunofluorescence, including the fact that pathologists prefer it largely because it allows integration of ‘phenotypic’ features in the IHC stain with the traditional morphologic features, long the ‘gold standard’ for diagnosis.

An important caveat is that the optical properties of the chromogens will affect the linearity and dynamic range of the assay. The Lambert-Beer law that underlies the unmixing approach applies only to pure absorbers. Some chromogens, most notably the popular brown DAB stain, exhibit scattering behavior similar to that of melanosomes. In fact, it can be impossible to separate DAB from melanin pigmentation spectrally, since their spectra arise from the same optical properties. However, in practice, this does not seem to pose insuperable problems, since linearity and reasonable dynamic range can be achieved using DAB approaches (41). Other chromogens, such as Vector Red, have been shown to have excellent linearity and dynamic range (42).

In addition to the specific molecular labeling procedure, a counterstain is almost always applied. Thus the challenge for quantitation begins with the unmixing of the chromogen (typically DAB) from the counterstain (typically hematoxylin). The latter pair can be successfully unmixed using simple RGB imagery if conversion to OD is performed (39), but other pairs may not be so amenable. One of the challenges (see below) is the accurate determination of the spectra of the chromogens as input values into the unmixing procedure. Small variations in the spectra chosen can have quite dramatic effects on the calculated abundance values. While in many cases it suffices to measure the spectrum of the isolated chromogens (single stain, no counterstain), we have found that it may be necessary to measure the spectrum of the chromogens in the actual sample, after all the staining procedures have been performed, since the spectra can be affected by the presence of other dyes and reagents.

### Multiplexing

Typically, only a single IHC-chromogen-antigen combination is used per slide; if more than one antigen is to be analyzed, serial sections are made and a different antibody is applied to each. This procedure benefits from simplified protocols and quality control regimens compared to multicolor techniques, but generates more slides and possibly more preparation steps than if the reagents are ‘multiplexed’ on a single slide. Moreover, multiple molecular events cannot be evaluated on a per-cell basis when parallel sections are employed, and this capability is very important in establishing the phenotype of individual tumor cells (e.g., lymphoma cells) distributed in a mixed cell population. Multicolor immunohistochemistry is thus an important goal, but is challenging to achieve. The prerequisite to quantitative accuracy in a multiple labeled section is lack of interference between the labels. Not only can one label physically block the successful labeling of the next antigen due to steric hindrance, but the various labeling procedures can be chemically incompatible. Suffice it to say that the performance of multiple labelings on a single specimen increases the demands for appropriate controls (43). Assuming that the labeling procedures have been performed satisfactorily, unmixing of 3 or more chromogens is entirely feasible (38,44) (Levenson, submitted). In addition, multiple chromogenic in situ hybridization signals can be combined with IHC (45, 46).

### Examples of Spectral Unmixing and Multiplexing

FIGS. 1 and 2 illustrate the application of spectral imaging to a determination of Ki67 levels in lymph node cells. The Ki67 antigen was visualized using DAB and the sample counterstained with hematoxylin (H). FIG. 1 shows the visual appearance of the sample (Panel A), which, like all the subsequent examples, was spectrally imaged using a Nuance multispectral imaging system. The unmixed DAB and hematoxylin channels are shown in Panels B and C. Note that the hematoxylin staining accurately recapitulates the dense staining of the mantle cells and the paler staining of the germinal center. The small box indicates the detail region highlighted in FIG. 2, which addresses the importance of accurately estimating the “pure” spectrum of the DAB for use in the unmixing procedure. Three different spectra for the DAB component were used as inputs into the unmixing procedure. If one simply captures the spectrum of a DAB-labeled nucleus (top row), unmixes and examines the hematoxylin channel, it can be seen that all of the absorbance (due to DAB plus hematoxylin) ends up in the DAB channel, and a white “hole” is seen in the DAB-positive regions in the H channel. The integrated intensity of the DAB-labeled nucleus is indicated. If one attempts to calculate the “pure” spectrum of the DAB by removing the H component, a variety of curves can be generated, depending on the nature of the algorithm used. The second row shows what happens if overcompensation occurs—in this case, some of the DAB signal remains in the H channel, leading to an overly intense H signal and an underestimation of the DAB intensity. Finally, if the DAB spectrum is correctly estimated, unmixing generates a clean partition of DAB and H signals, in which the H intensity of the labeled nucleus is essentially indistinguishable from that of its neighbors. The integrated intensities of the DAB label in the circled nucleus varied by more than 2-fold depending on the spectra chosen, illustrating the quantitative importance of correct unmixing. Of course, the importance of using appropriate spectra for the unmixing process only increases with the number of chromogens being considered simultaneously.

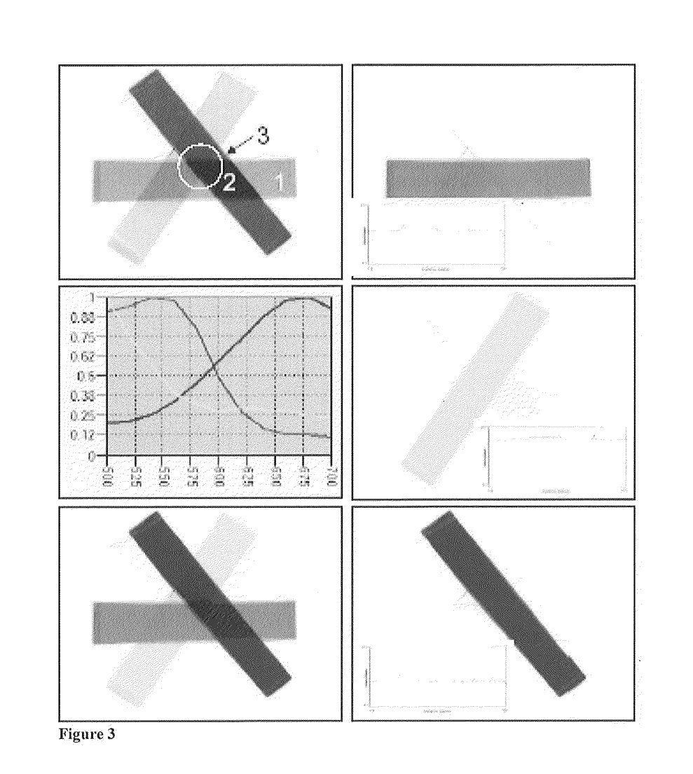

FIG. 3 is intended to demonstrate that 3-color unmixing is feasible, using 3 strips of colored plastic arranged so that all possible combinations of single, double and triple mixtures are captured. The spectra of the individual strips are shown, as are the unmixed images for each strip separately (pseudocolored according to the color of the spectral library curves in Panel B), along with intensity profiles along each strip. As can be seen, calculated absorbance values of each strip are unaffected by the presence of the other absorbers.

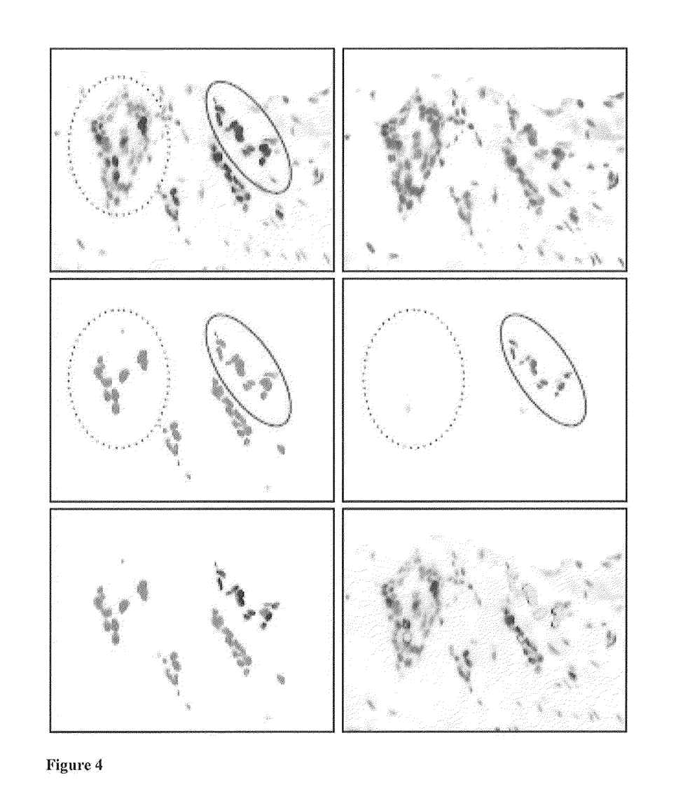

Finally, FIG. 4 illustrates the application of unmixing to a histological section of formalin-fixed, paraffin-embedded breast tissue containing both non-malignant and invasive breast epithelial cells, stained for ER and PR, and counterstained with hematoxylin. This example has considerable current relevance because the detection and evaluation of nuclear positivity of breast cancer steroid hormone receptors can affect choice of treatment and is useful in predicting patient outcomes (7,47). Receptor levels are currently evaluated manually, typically using a 0 to 3+ grading system and/or a simple visual estimate of the number of positive nuclei in a relevant cellular population. In this example, ER and PR antigens were visualized with DAB and Vulcan Red chromogens and counterstained with hematoxylin (H). The 6 panels illustrate the original visual appearance, and after unmixing the H channel (which can be used to identify the nuclear compartment for quantitative purposes), and separate channels for ER and PR (green and red, respectively). The dotted oval identifies a region of presumptively normal epithelium, and the red oval a region of invasive ductal carcinoma. The bottom panels show an overlay of the green and red channels, and finally, a depiction of the original image with ER-PR double-positive cells is indicated using a yellow mask. It is striking that the normal and the malignant regions exhibit different co-localization patterns (normal, ˜5%; malignant, ˜55%, on a pixel-wise basis).

The biological significance of this and other patterns of markers revealed quantitatively on a per cell basis is currently unknown. What is important is that now there are tools to explore molecular interrelationships in individual cells using multicolor IHC-based techniques, with the potential for quantifiable results, pre-requisites for the beginnings of ‘Molecular Morphology’ (48).

In conclusion, quantitative immunohistochemistry is not a distant mirage, but is within our grasp. It will require careful attention to the pre-imaging components, including provision of quantitative standards (19) to be included in the entire sample processing pathway, and attention to all parameters of sample acquisition, fixation, and staining, with good QC procedures in place for each probe singly and in combination. For multiplexing, the interaction of one antibody-label combination on all the others must be understood and controlled, and choice of chromogen and counterstains will affect both the visual and quantitative results. Finally, the imaging component has to be carefully performed, with appropriate sensors, exemplified by multispectral, reliable and validated unmixing algorithms. In addition, and not discussed above, it will be essential to incorporate appropriate downstream image analysis and quantification approaches that accurately report molecular events on a per-pixel, per-cell, or per ‘relevant tissue component’ basis, as appropriate. Ultimately, especially for clinical applications, this task becomes a systems-problem, in which the entire process, from sample acquisition to reporting and interpretation needs to be integrated, standardized (11,19,49), and to the greatest extent possible, automated.

### REFERENCES

1. Horobin R W, Kiernan J A. Editors. Conn's Biological Stains. 10^{th} Edition. Biological Stain Commission, Bios Publishers. Oxford, 2002
2. Becker R L. Standardization and quality control of quantitative microscopy in pathology. J. Cell. Biochem. 1993; 17G: 199-204
3. The Non-Hodgkin's-Lymphoma Classification Project. Clinical Evaluation of the International Lymphoma Group Classification of Non-Hodgkin's Lymphoma. Blood. 1997; 89: 3909-3918
4. Taylor C R. Quality assurance and standardization in immunohistochemistry. A proposal for the annual meeting of the Biological Stain Commission, June, 1991. Biotechnic. Histochem. 1992 (a); 67:110-117
5. Taylor C R, Cote R J. Immunomicroscopy; A Diagnostic Tool for The Surgical Pathologist. 3^{rd} Edition. Elsevier, Edinburgh, 2005
6. Grube D. Constants and variables in immunohistochemistry. Arch. Histol. Cytol. 2004; 67:115-134
7. Diaz L K, Sneige N. Estrogen receptor analysis for breast cancer: current issues and keys to increasing testing accuracy. Adv. Anat. Pathol. 2005; 12(1):10-9
8. Seidal T, Balaton A J, Battifora H. Interpretation and quantification of immunostains. Am. J. Surg. Patholo. 2001; 25: 1204-1207
9. Umemura S, Itoh H, Serizawa A et al. Immunohistochemical evaluation of hormone receptors in breast cancer: which scoring system is suitable for highly sensitive procedures. Appl. Immunohistochem. Mol. Morph. 2004; 12: 8-13
10. Umemura S, Osamura R Y. Utility of Immunohistochemistry in breast cancer practice. Breast Cancer. 2004; 11: 334-338
11. Leong A S-Y. Quantitation in Immunohistology: Fact or Fiction? A Discussion of Variables That Influence Results. Appl. Immunohistochem. Mol. Morph. 2004; 12:1-7
12. Dapson R W, Feldman A T, Wolfe D. Lessons In Immunohistochemistry. Anatech, Battle Creek, Mich., 2005
13. Nadji M, Nassiri M, Vinceck V et al. Immunohistochemistry of tissue prepared by a molecular friendly fixation and processing system. Appl. Immunohistochem. Mol. Morph. 2006; 115:814-822
14. Shi S R, Cote R J, Young L L, Taylor C R. Antigen retrieval immunohistochemistry: practice and development. J. Histotechnol. 1997; 20: 145-154
15. Shi S R, Cote R J, Chaiwun B et al. Standardization of immunohistochemistry based on antigen retrieval technique for routine formalin-fixed tissue sections. Appl. Immunohistochem. Mol. Morph. 1998; 6: 89-96
16. Shi S R, Gu J, Taylor C R. Antigen Retrieval Techniques: Appl. Immunohistochem. Mol. Morph. Eaton Publishing, Natick, Mass., 2000
17. Taylor C R. Report from the Biological Stain Commission: FDA Issues Final Rule for Classification/Reclassification of Immunochemistry (IHC) Reagents and Kits. Biotechnic. Histochem. 1998; 73:175-177
18. Taylor C R. An Exaltation of Experts; concerted efforts in the standardization of Immunohistochemistry. Appl. Immunohistochem. 1993; 1: 232-243
19. Taylor C R. Standardization in immunohistochemistry: the role of antigen retrieval in molecular morphology. Biotechnic. Histochem. 2006 (in press).
20. Sompuram S R, Kodola V, Zhang J et al. A novel quality control slide for quantitative immunohistochemistry testing. J. Histochem. Cytochem. 2002; 50:1425-1433
21. Shi S R, Liu C, Perez J, et al. Protein-embedding technique: a potential approach to standardization of immunohistochemistry. J. Histochem. Cytochem. 2005; 53: 1167-1170
22. Rhodes A, Jasani B, Couturier J, McKinley M J, Morgan J M, Dodson A R, Navabi H, Miller K D, Balaton A J. A formalin-fixed, paraffin-processed cell line standard for quality control of immunohistochemical assay of HER-2/neu expression in breast cancer. Am. J. Clin. Pathol. 2002; 117(1):81-9
23. Shi S R, Cote R J, Liu et al. A modified reduced temperature antigen retrieval protocol effective for use with polyclonal antibody to Cox2 (PG 27). Appl. Immunohistochem. Mol. Morph. 2002; 10:368-373
24. Cross S S. Observer accuracy in estimating proportions in images: implications for the semi-quantitative assessment of staining reactions and a proposal for a new system. J. Clin. Pathol. 2001; 54: 385-390
25. Lehr H A, Jacobs T W, Yaziji H et al. Quantitative evaluation of HER-2/neu status in breast cancer by fluorescence in situ hybridization and by immunohistochemistry with image analysis. Am. J. Clin. Pathol. 2001; 115: 814-822
26. Foucar E and Foucar M K. Error in Anatomic Pathology. In Foucar MK. Bone Marrow Pathology. 2nd. Edition. ASCP Press, Chicago, 2000
27. Sirota R L, Error and Error Reduction in Surgical Pathology. Arch. Pathol. Lab. Med. 2005: 129: 1228-1233
28. Rhodes A, Jasani B, Balaton A J, Miller K D. Immunohistochemical demonstration of estrogen and progesterone receptors: correlation of standards achieved on in house tumours with that achieved on external quality assessment material in over 150 laboratories from 26 countries. J. Clin. Pathol. 2000; 53: 292-301
29. Hatanaka Y, Hashizume K, Kamihara Y et al. Quantitative immunohistochemical evaluation of HER2/neu expression with HercepTest TM in breast carcinoma by image analysis. Pathol International. 2001; 51: 33-36
30. Rhodes A, Jasani B, Anderson E et al. Evaluation of Her2/neu immunohistochemical assays sensitivity and scoring on formalin-fixed and paraffin-processed cell lines and breast tumors: a comparative study involving results from laboratories in 21 countries. Am. J. Clin. Pathol. 2002; 118: 408-417
31. Camp R L, Chung G G, Rimm D L, Automated subcellular localization and quantification of protein expression in tissue microarrays. Nat. Med. 2002; 8(11):1323-7
32. Rubin M A, Zerkowski M P, Camp R L et al. Quantitative determination of expression of the prostate cancer protein alpha-methylacyl-CoA racemase using automated quantitative analysis (AQUA): a novel paradigm for automated and continuous biomarker measurements. Am. J. Pathol. 2004; 164(3):831-40
33. Bengtsson E. Computerized Cell Image Analysis: Past, Present, and Future. Image Analysis: 13th Scandinavian Conference, SCIA 2003 Halmstad, Sweden, Jun. 29-Jul. 2, 2003 Proceedings Volume 2749/2003, Lecture Notes in Computer Science Berlin/Heidelberg: Springer 2003; 395-407
34. Bearman G, Levenson R. Biological Imaging Spectroscopy. In: Vo-Dinh T, editor. Biomedical Photonics Handbook. Boca Raton: CRC Press, 2003; 818-26
35. Barber P R, Vojnovic B, Atkin G et al. Applications of cost-effective spectral imaging microscopy in cancer research. J. Phys. D: Appl. Phys. 2003; 36:1729-1738
36. Levenson R M. Spectral imaging perspective on cytomics. Cytometry, Part A. 2006; in press
37. Mansfield J R, Levenson R M. Spectral imaging in biology and medicine: Slices of life. Cytometry, Part A. 2006; in press
38. Zhou R, Hammond E H, Parker D L. A multiple wavelength algorithm in color image analysis and its applications in stain decomposition in microscopy images. Med Phys 1997; 23(12):1977-1986
39. Ruifrok A C, Johnston D A. Quantification of histochemical staining by color deconvolution. Anal. Quant. Cytol. Histol. 2001; 23(4):291-9
40. Farkas D L, Du C, Fisher G W et al. Non-invasive image acquisition and advanced processing in optical bioimaging. Comput Med Imaging Graph 1998; 22(2):89-102
41. Matkowskyj K A, Cox R, Jensen R T et al. Quantitative immunohistochemistry by measuring cumulative signal strength accurately measures receptor number. J. Histochem. Cytochem. 2003; 51(2):205-14
42. Ermert L, Hocke A C, Duncker H R et al. Comparison of different detection methods in quantitative microdensitometry. Am. J. Pathol. 2001; 158(2):407-17
43. Szeszel M K, Crisman C L, Crow L et al. Quantifying estrogen and progesterone receptor expression in breast cancer by digital imaging. J. Histochem. Cytochem. 2005; 53(6):753-62
44. Levenson R, Cronin P J, Pankratov K P. Spectral imaging for brightfield microscopy. Proc SPIE 2003; 4959:27-33
45. Speel E J, Jansen M P, Ramaekers F C et al. A novel triple-color detection procedure for brightfield microscopy, combining in situ hybridization with immunocytochemistry. J. Histochem. Cytochem. 1994; 42(10):1299-1307
46. Macville M V E, Ried T. Multicolor spectral imaging of chromogenic dyes in cytological specimen. J. Histochem. Cytochem. 1998
47. Press M F, Sauter G, Bernstein L et al. Diagnostic evaluation of HER-2 as a molecular target: an assessment of accuracy and reproducibility of laboratory testing in large, prospective, randomized clinical trials. Clin. Cancer. Res. 2005; 11(18):6598-607
48. Taylor C R. Immunohistochemistry for the age of molecular morphology. Appl. Immunohistochem. Mol. Morph. 2001; 9:1-2
49. O'Leary T J. Standardization in Immunohistochemistry. Appl. Immunohistochem. Mol. Morph. 2001; 9: 3-8

### Example Ii

### Quantifiable Internal Reference Standards for Immunohistochemistry; the Measurement of Quantity by Weight

Absent uniform sample preparation for formalin paraffin tissues, and absent available tissue reference standards, It is one aspect of the present invention that selected defined analytes (proteins) present intrinsically within tissues may be employed as quantifiable internal reference standards, against which sample quality can be directly assessed and key analytes directly quantified by immunohistochemistry. The panel of ‘quantifiable internal reference standards’ for FFPE tissues will serve to control for the variable effects of sample preparation, and simultaneously would provide a reference base for calibration and quantitative analysis of specific analytes.

### Introduction and Background

The poor reproducibility of immunohistochemical (IHC) and molecular methods as applied to formalin fixed paraffin embedded (FFPE) tissue sections, is now recognized as a major impediment to basic research, clinical trials and direct patient care.

In the year 2006, cancer still is diagnosed by the surgical pathologist with his/her microscope using methods that essentially are unchanged over 150 years, from the time that the first histology course was conducted by John Hughes Bennett at Edinburgh, in 1842, and the first major textbook of surgical pathology was drafted by Rudolph Virchow in 1858 (1). That this remains true today, in an era viewed by the public, by politicians and by many scientists, as the era of molecular biology and genetics, is astonishing (2). While several factors contribute, the primary reason for this anachronism is simple. The translation of ‘molecular methods’ from the bench to ‘routine’ diagnostic practice in surgical pathology has been greatly hindered by the fact that the usual method of sample preparation for tissue is formalin fixation and paraffin embedment (FFPE). This venerable approach may be satisfactory for the preservation of morphologic detail, but it is certainly not the method of choice for molecular or immunologic assays (including immunohistochemistry IHC, and in situ hybridization—ISH). The enormous variation in protocols employed for FFPE among different laboratories, or within the same laboratory from specimen to specimen, compounds the problem, and contributes to the current poor reproducibility of these methods.

Over the past two decades many investigators have addressed different aspects of this problem, focusing upon improved sample preparation (fixation), more effective methods of antigen retrieval, and the development of external reference standards or controls. To date, these approaches have failed to produce an overall system of IHC that assures uniform high quality, with a level of reproducibility and reliability, sufficient to allow the possibility of true quantitative analysis.

Some Broad Conclusions are Possible:

for reproducibility of IHC staining techniques overall—current reagents and protocols are probably satisfactory; significant further improvement is dependent upon resolution of the problems of sample preparation, coupled with availability of standard reference materials.

for sample preparation—the scientific aspects of developing a new fixative are challenging and not yet solved; more importantly the logistical and economic obstacles to replacing formalin, worldwide, with something better, even if it became available, are formidable.

for reference standards—the scientific challenges of developing either FFPE cell line blocks, or ‘faux’ tissues, or protein (or RNA) standards are significant, but again are dwarfed by the logistical and economic obstacles of manufacture, distribution, and inclusion of any external reference material into essentially all FFPE blocks in all laboratories going forward.

Considering the extent of both scientific and economic challenges, the author therefore accepts the following as practical facts:

i. methods of sample preparation of tissues (including fixation) for surgical pathology are unlikely be standardized in the next decade;

ii. universal tissue reference standards will not be available in the foreseeable future;

iii. the scientific and patient care communities will therefore be forced to continue to work with FFPE tissues, in spite of manifold drawbacks;

iv. attempts to standardize IHC on FFPE tissues to a degree that permits quantification are doomed to fail in the absence of reference materials that allow absolute measurement of performance (including reproducibility) of the process as a whole.

These conclusions apply to immunohistochemical (IHC) and in situ hybridization (ISH) methods applied to FFPE tissue sections, and equally to all ‘molecular’ analyses of proteins, RNA or DNA extracted from FFPE blocks. Even if the problems of sample preparation could be solved, existing archival blocks would still not be addressable for quantitative analysis by any of these methods, and the numerous existing clinical trials that are dependent on data from archival FFPE materials would not be advantaged.

“Anatomic pathology changed little in the 100 years preceding 1970. Sequestered in a technologic limbo, it remained relatively untouched by the new methodologies and automated systems that revolutionized the clinical laboratory. The histology laboratory performing only a few simple stains, thereby escaped the rigors of quality assurance in general, and quality control in particular. To dip a slide in hematoxylin for a few minutes, then briefly differentiate it in alcohol, until it looks ‘about right’ to the technologist and ‘makes the pathologist happy’ may suffice an H&E stain, but applied to immunohistochemistry it is a recipe for disaster” (6).

More than a decade has passed since these words were put to paper, and at last there are signs that “the times they are a-changin” (7). As ever, necessity may be the mother of invention. The current burgeoning necessity, spawned of a need for clinical accuracy, is that an IHC ‘stain’ shall provide validated quantifiable results. This necessity is proving to be a potent driver of change, elevating such mundane issues as ‘sample preparation’ and ‘standardization of IHC (and ISH, in situ hybridization) stains’ from the status of obscure academic pursuits to real practical problems, demanding of an answer.

Anatomic pathology (surgical pathology, histopathology) is subjective to a degree, based upon pattern recognition and experience (8,9). Quantitative elements often are present, albeit, subliminally, as in gauging the degree of hyperchromatism, or eosinophilia, or even counting mitoses per high power field, but these evaluations are not strictly rule based, not easily reproduced, and they are not quantitative. The usual histopathologic stains [biological stains and aniline dyes, see Conn's Biologic Stains (10)] are qualitative in nature and difficult to perform reproducibly, in terms of intensity of color (stain), from cell to cell and from section to section (different tissues on different days).

Immunohistochemical ‘stains’ are potentially very different, in that they do contain the inherent elements necessary to provide quantitative data, because each IHC ‘stain’ is in essence a tissue based ‘immunoassay’, that is performed in situ on the tissue section. An IHC ‘stain’ in principle, and in major elements of practice, is identical to an ELISA (enzyme linked immunosorbent assay) test performed in the clinical laboratory, and ELISA based tests are widely recognized as being truly quantitative, if properly performed. Exactly the same reagents that are employed in an ELISA test on serum, for example an assay for insulin, may be employed to perform an IHC stain for insulin in a paraffin section. It is a curious oversight of pathologists, that the principles and reagents used in one environment (serum—ELISA) are universally accepted as providing a strictly quantitative result, but when applied to a tissue section (IHC), constitute only a ‘stain’, that at best may be employed in some form of semi-quantitative assay, with the intrinsic shortcomings that the term implies.

One object of this invention is to examine the reasons for this conceptual divide. A second goal is to address those aspects of the IHC method that have to date relegated it to the rank of a mere stain, as opposed to a tissue based immunoassay, with a quantitative outcome.

### The Immunohistochemical Stain

More then a decade ago the Biologic Stain Commission, in conjunction with the FDA, provided critical leadership in beginning to address the ‘standardization’ of IHC methods (11,12). Several sponsored conferences focused upon the poor reproducibility of IHC staining methods, prompting a thorough analysis of the possible causal factors. One result was the formulation of the “Total Test Approach”, borrowed directly from the rigorous and comprehensive test protocols used in quantitative assays in the clinical laboratory. In the ‘Total Test Approach’, all aspects of the assay are addressed; pre-analytic, analytic, and post-analytic, including interpreting and reporting of the results (Table 1).

TABLE 1
The Total Test; an IHC (or ISH) stain managed in the same rigorous
manner as a clinical laboratory analysis

| | |
|---|---|
| Pre-analytic | Test selection |
| | Specimen type, acquisition, transport time* |
| | Fixation, type and time* |
| | Processing, temperature* |
| Analytic | Antigen retrieval procedure* |
| | Protocol; control selection |
| | Reagent validation |
| | Technician training/certification |
| | Laboratory certification |
| Post analytic | Control performance |
| | Results |
| | Interpretation/Reporting |
| | Pathologist, experience and CME |

*highly variable elements of in the analytic process Modified from Taylor (11, 13)

While the entire constellation of issues contributing to the performance of an IHC stain was considered (Table 1), the outcome was inevitably somewhat pragmatic, with a focus upon correcting those parts of the process that were most amenable to correction. The quality of reagents was at that time (1992) highly variable, and the validation of reagents by both manufacturers and laboratories left much to be desired. Acting in concert, the BSC and the FDA made recommendations to manufacturers, a number of whom participated in the deliberations. The outcome was an improvement in format and content of package inserts, particularly greater stringency in the claims of manufacturers as to how the their reagents could (and should) be used in diagnostic pathology (11,13).

At about the same time, a second trend was emerging in respect to the practical application of IHC staining, namely the demonstration of prognostic and predictive markers at a cellular level. The availability of numerous new (monoclonal) antibodies facilitated the detection in tissue sections of a variety of molecules that were not directly lineage related, but rather were reflective of the metabolic status of the cell, whether in terms of the phase of cell cycle, or the degree of expression of receptors involved in cell growth. Estrogen receptor (ER) and progesterone receptor (PR) were among the first of these to assume clinical significance, with respect to prognosis and therapeutic response, in this instance in breast cancer (14,15,16). Estimation of Her 2/neu expression by IHC presented similar challenges and soon came to be of paramount importance, with the advent of a therapeutic monoclonal antibody directed against the HER 2 receptor (review, 17). While semi-quantitative IHC studies had been described prior to this time, the shift towards the use of IHC to demonstrate prognostic and ‘therapeutic’ markers, added real urgency to the need for true quantitative methods. The inherent difficulties are well recognized for ER and PR (18,19,20), where both methodology and scoring vagaries contribute to error, and where uniform reference standards are not available. The problem is arguably even greater for Her 2 (21,22), where the FDA-approved Dako kit (HercepTest, Dako, Glostrup, Denmark, www.dakousa.com) does contain a cell line standard and includes instruction about how to read the result. Even with these provisions, scoring of the same cases for HER 2 expression by residents and pathologists shows significant variation, leaving room for improvement.

### Towards a Solution

Current approaches to improving the overall quality of IHC staining methods have focused primarily upon sample preparation and quality control or reference standards. The focus of the National Institutes of Health RFA alluded to previously (RFA CA-07-003) is similar: ‘enhancement or adaptation of sample preparation methodologies—development of assays to assess sample quality’. This rationale is at first sight sound, in that if these two problem areas are resolved, then developing greater reproducibility of IHC staining should be relatively straightforward. However, it is the view of the author that there is no realistic solution in sight for these key problems,

### Sample Preparation

The ‘Total Test Approach’ served to highlight the importance of specimen acquisition and sample preparation in contributing to the (lack of) quality of the end result of an IHC stain, a deficiency that in turn hampered serious efforts at quantification. In the Clinical Laboratory the response to a specimen that is incorrectly prepared (e.g., in the wrong anticoagulant, or outside of the specified transportation time), is that the specimen (and test) is rejected; not so in surgical pathology, where the general response is to an improperly or poorly fixed specimen is to carry on regardless, seen almost as a challenge to get an acceptable H&E stain, usually without even a notation of a major variance in sample preparation. Where morphologic quality is the only arbiter of ‘adequate’ processing and handling (for FFPE), the aforementioned response has sufficed for more than a hundred years, but today for IHC and ISH assays, it does not. Now, as IHC methods are being employed in attempts to ‘measure’ prognostic markers, the traditional cavalier approach to sample preparation (FFPE) has emerged as a critical problem. Today the question is “Exactly how much of the analyte (e.g., ER, HER 2) is present?” Not merely “Is it there, or not there?”, as might be sufficient in applying IHC to identify a lineage related marker (e.g. keratin in a putative ‘epithelial’ cell). The problem reached national attention with the increasing use of IHC findings, as entry criteria for patients into clinical trials (exemplified by staining for Her 2 or CD20, as indicators of possible effectiveness of monoclonal antibody therapy). A NIST (National Institute of Standards) sponsored workshop in Washington (23) cataloged the existing problems, but found no solution at hand.

Sample preparation (including fixation) had been considered by the BSC (as in Table 1), but the problem was deemed complex, without obvious and feasible means of immediate improvement. Over the succeeding decade, ‘fixing the fixation problem’ was rendered less urgent by the discovery and dissemination of the antigen retrieval (AR) technique (reviews 24,25,26), which had the practical effect that ‘useful’ IHC staining could be readily achieved by many laboratories for many molecules. Efforts to replace formalin with a new fixative, dubbed by some as more ‘molecular friendly’ (27), continued, but seemed less urgent. New fixatives, or new formulations of old fixatives, continue to be described, and the prototypic data do indeed suggest that one (or more) of them may be superior to formalin with regard to the capability for subsequent demonstration of tissue analytes (proteins, RNA and DNA) (review, 20). However, even if these claims are granted, and some continue to protest that the fine morphology is ‘different’, the logistics of converting to a new fixative and new processing method, worldwide, are extremely demanding. History would suggest that if a change did occur, it would occur slowly, randomly, and non-uniformly, and for a time reproducibility would be worse, not better. Also, even if a new fixation and processing method were to be adopted universally, their existence would not enhance access to the huge wealth of data residing in archival FFPE tissues throughout the world, that must form the basis for diagnosis and entry into clinical trials for years to come.

TABLE 2
Summary of desirable characteristics of a ‘reference standard’ that
would provide a basis for accurate quantification of IHC (or ISH) (28)
Immunohistochemical Reference Standard - requirements for calibration of
quantitative IHC methods, by analogy with defined standards in clinical
laboratories

It must be subjected to the same rigors of sample preparation as the
‘test’ tissue, to include any effects of tissue ischemia, fixation and
processing.
It must be integrated into all phases of the test (assay) protocol,
including evaluation of the result.
It should contain a known amount of the analyte(s) subject to assay.
It should be universally available.
It should be inexhaustible.
It should be inexpensive.

### Assay Quality Control—a Reference Standard

The development of a universal external reference standard, sharing the characteristics of calibration standards employed in clinical pathology (Table 2) (28), has encountered difficulties, both scientific and practical. In addition to the commonly employed ‘positive control’ sections, and tissue micro-arrays (29), different investigators have pursued cell lines or cell line blocks (30), ‘faux’ tissues or histoids [(2) p 35, FIG.—1-27], and protein ‘spots’ or deposits (31,32,33). The use of cell lines per se has of course been employed for a FDA approved Her2 ‘staining test’ kit (Dako, HerCept test), with results that are semi-quantitative and, as already noted, may be difficult to reproduce among laboratories and pathologists. With ‘faux’ tissues or cell line blocks the practical issues of scale up to a commercial level of production and distribution in a form that could incorporated in all stages of sample preparation (FFPE), are at present insurmountable, primarily for economic reasons. The problems of developing purified protein standards, are both similar and different; similar in that the logistics of distributing any reference standard and incorporating the appropriate standard into FFPE blocks routinely (for each different stain) are daunting; different in that the technical challenges to preparing standard protein deposits that will survive FFPE have been explored with limited success (33,34). As currently constituted the usual positive controls, cell lines, or sections, are in reality ‘qualitative’ controls. They are selected to contain sufficient analyte to produce (usually) intense staining, but exactly how much of the analyte is present in the ‘control’ is entirely unknown. The best, therefore, that can be achieved is a semi-quantitative result, comparing one section against others, and concluding that staining is more or less intense, or more or less extensive, with the assumption that this relates to the relative amounts of analyte present. This approach fails in significant ways to meet the required characteristics set forth as Table 2, critically, for the purposes of quantification, in lacking data as to the measured amount of the test analyte present in the control.

The idea of utilizing ‘internal controls’ for IHC dates back to the first routine immunoperoxidase stains of formalin paraffin tissues (35), exemplified by the use of plasma cell staining in evaluating whether a stain for kappa chain has ‘worked’, or not [reviewed in ‘Immunomicroscopy’ (2)]. There is also a precedent in the use of internal controls to assess the extent of overall ‘loss of antigenicity’ following FFPE, by staining for vimentin, which may be regarded as ‘formalin sensitive’ and is present in almost all tissue samples (36). The implication is that the degree (intensity) to which vimentin stains, or does not stain, may serve as an indicator (‘reporter molecule’) of the expected degree of staining of other proteins (analytes). However, these internal controls were used as purely qualitative (not quantitative) controls for sample processing.

Some more recent hint as to the direction that might be taken is gleaned from the work of Dr. R. Singer and colleagues (37), who have commenced a collaboration with our group at USC, with the goal of identifying quantifiable internal standards for FFPE tissues, both proteins and RNA. Singer's group described a method, dubbed RNA peT-FISH (paraffin embedded Tissue) for demonstrating RNA gene expression profiles in individual cells in FFPE sections. The method proved effective on a variety of FFPE tissues, yielding predictive quantitative gene expression signatures. In effect, the method employs ubiquitous house keeping gene RNAs as internal reference standards, that in theory may be developed to provide the basis of a validated quantitative ISH method.

### Quantifiable Internal Reference Standards for IHC

For a reference standard to be effective as a Quantifiable Internal Reference Standard, it is preferably present in the same FFPE section, alongside the antigen under study (test analyte). In accordance with one embodiment of the present invention, an IHC stain (read—‘assay’) for which the goal is a quantifiable result, is in the form of standardized controlled ‘double IHC stain reaction’, including a ‘stain’ for the unknown ‘test’ analyte, and a second ‘stain’ for an internal reference analyte. The amount present of the unknown ‘test’ analyte (protein) is then measured by comparison of the intensity of IHC staining of the ‘test’ analyte with the intensity of staining of the reference analyte, using preferably, validated quantitative IHC protocols and computer assisted image analysis, as by comparative quantitative spectral imaging (28).

As described below and expounded in supplemental data filing (6-30-10) Several candidate reference analytes were selected on the basis of their presence in relatively constant amounts in specific cell types that are easily recognized and widely distributed (such as endothelial cells or lymphocytes). This predicate is easily tested, as described by Taylor and Becker 2011. In establishing a standard, the absolute amount of the candidate reference analyte in fresh tissue was determined by experiment using independent methods, for example, on a per cell basis. (cite paper by Becker and Taylor e mailed to you) It was necessary to establish the extent to which the reference analyte(s) is preserved following FFPE with optimized antigen retrieval. For each new QIRS these data will be derived experimentally and may be expressed as a ‘fixation coefficient’ (F×C), encoding the relationship of the absolute amount of analyte (antigen) present in the fresh tissue (cell) and the intensity of the corresponding IHC signal, with the amount of analyte present in the FFPE tissue and the intensity of its IHC signal, by identical IHC protocols. Similar data are then collected, again by experiment, for various test analytes for which a quantitative result is required (e.g., ER, Her2), relating the experimentally derived ‘fixation coefficient’ for each potential test analyte with that established for one or more reference analytes, that show similar behavior when subject to FFPE. With such data in hand, measurement of the reference analyte IHC signal and the test analyte IHC signal on a double stained slide allows accurate calculation of the amount present (e.g., on a per cell basis), with far greater precision than is achievable by current ‘semi-quantitative’ scoring methods.

This QIRS approach also exploits the idea that the adverse effects of different FFPE methods during sample preparation may be minimized by the use of an optimized-AR protocol, resulting in improved reproducibility of IHC staining, presumably reflective of some consistency in recovery of antigen. This strategy was pioneered by our group (38), and has been proven effective for qualitative IHC studies among different laboratories. It offers the possibility that for one of more candidate reference analytes the ‘fixation coefficient’ may show acceptable consistency across the usual variations encountered in formalin fixation and paraffin embedment. A perfect answer is not expected, merely something better than the ‘uncontrolled controls’ available to us today. Ultimately it should be possible to provide a reliable measurement (by calculation) of the amount of unknown test analyte present in the cells/tissue prior to the initiation of sample preparation (i.e., when it was removed from the patient).

While absolute accuracy is not envisaged, it is at least possible that results can be achieved that are superior to current semi-quantitative IHC measurements, that make little attempt to control for vagaries in sample preparation, and lack any objective (quantifiable) reference standard whatsoever (Taylor and Becker 2011). Once a ‘quantifiable internal reference standard’ is established in a cell adjacent to another cell containing the ‘test’ analyte within an FFPE section, then other confounding issues, such as variation in section thickness, or the exact plane of transection of individual cells, can be addressed, in the manner of ‘background noise’, by computer assisted image analysis systems.

In establishing protein based standards, encouragement may be drawn from the application of a similar rationale to the development of internal RNA reference standards, in the design of the peT-FISH method for FFPE tissues, using house keeping gene RNAs as internal reference standards, as already described (37). Also there is the analogy of the standardized RT-PCR (StaRT PCR) method, which can be rendered quantitative by the use of internal actin RNA (widely distributed in different cells) as the reference control (39). We have successfully employed this approach in our laboratories to quantify transcripts in bladder cancer cell lines and tumor tissues, and demonstrated its superior reproducibility and consistency in relation to real time PCR (40).

### Conversion of an IHC ‘Stain’ to an IHC ‘Analysis’

The availability of effective, reliable, quantitative IHC and ISH methods would allow visualization and ultra-cellular localization of key analytes, important to the diagnosis and prognosis of cancer, in conjunction with traditional surgical morphology criteria used for cell recognition and diagnosis. The potential offered by this combined dual capability is becoming known as Molecular Morphology. Few would argue against the notion that surgical pathology (particularly cancer diagnosis) has been transformed by the advent of IHC methods. Rendering the method both reproducible and quantitative would mean that both IHC and ISH ‘stains’ would function not just stains, but as tissue based assays, to be managed with the same rigor as any other immune based quantitative assay in the laboratory.

### Quantitative Molecular Morphology

Ultimately it would be possible reliably to measure RNA and protein, the end products of gene action, in situ within individual cells, leading to new criteria for cancer diagnosis and prognosis. In research the significance is profound, in that evaluation of gene activity, by the quantifiable demonstration of RNA expression and protein production, would allow scientists (read—pathologists) to gain information at the molecular level regarding the functioning of genes, not just their presence. The combination of these capabilities, for localization and quantification at a sub-cellular level, will open new fields of study, with regard to the pathogenesis of disease in general, and cancer in particular. If successful, it will provide the basis for establishing Quantitative Molecular Morphology (the combination of quantitative molecular and morphologic criteria) as the method for cancer diagnosis, prognosis and therapy selection. More important than any of these potential gains, is the possibility that the development of these methods will change the mindset of pathologists, from dealing simply with stains and patterns, to a modality that allows for the performance of direct quantitative assays on individual cells in tissue sections.

### REFERENCES

(1) Rudolph Virchow. Die Cellularpathologie. Verlag von August Hirschwald, Berlin, 1858.
(2) Taylor C R and Cote R J. Immunomicroscopy; A Diagnostic Tool for The Surgical Pathologist. 3^{rd} edition. Elsevier, Edinburgh. 2005.
(3) Doyle Sir Arthur Conan. Sherlock Holmes, in the “Red-headed League”. Ed. H Greenhough Smith, Newnes, London. Strand Magazine. August: 1891.
(4) Doyle Sir Arthur Conan. Sherlock Holmes, in the “Sign of Four. Spencer Blackett, London (also in Lipincott's Magazine, same year). 1890.
(5) De Bono, Edward. Lateral Thinking. Pelican Books. London. 1970.
(6) Taylor C R. An Exaltation of experts: concerted efforts in the standardization of immunohistochemistry. Appl Immunohistochem 1993; 1:232-243.
(7) Dylan Bob (Zimmerman, Robert Allen). The Times They Are A-changin’. Columbia Studios, New York. 1964.
(8) Hensen D E. Studies in observer variation. Arch Pathol Lab Med. 1991; 115, 991-992.
(9) Foucar E and Foucar M K. Error in Anatomic Pathology. In Foucar MK. Bone Marrow Pathology. 2nd Edition. ASCP Press, Chicago, 2000.
(10) Horobin R W and Kiernan J A. Editors. Conn's Biological Stains. 10^{th} edition. Biological Stain Commission, Bios publishers. Oxford. 2002.
(11) Taylor C R. Quality assurance and standardization in the immunohistochemistry. A proposal for the annual meeting of the Biological Stain Commission, June 1991, Biotech Histotech. 1992; 67:110-117.
(12) Taylor C R. Report from the Biological Stain Commission: FDA Issues Final Rule for Classification/Reclassification of Immunochemistry (IHC) Reagents and Kits. Biotechnic & Histochem. 1998; 73:175-177.
(13) Taylor C R. Standardization in immunohistochemistry: the role of antigen retrieval in molecular morphology. Biotechnics and Histochemistry. 2006 (in press).
(14) Henry J A, McCarthy A L, Angus B et al. Prognostic significance of Estrogen Regulated Protein, Cathepsin D, in Breast Cancer. Cancer. 1990; 65: 265-271.
(15) Elias J M, Cartun R A, England D M, et al. Interlaboratory Comparison of Estrogen Receptor Analysis in Paraffin Sectitons by a Monoclonal Antibody to Estrophilin (H222). J. Histotechnol. 1993, 16: 57-63.
(16) Battifora H, Mehta P, Ahn C. Oestrogen receptor immunohistochemical assay in paraffin-embedded tissue: a better gold standard? Appl Immunohistochem. 1993; 1: 39-45.
(17) Piccart M, Lorisch c, Di Leo A et al. The predictive value of HER 2 in breast cancer. Oncology. 2001; 61 (suppl. 2):73-82.
(18) Rhodes A, Jasani B, Balaton A J, Miller K D. Immunohistochemical demonstration of estrogen and progesterone receptors: correlation of standards achieved on in house tumours with that achieved on external quality assessment material in over 150 laboratories from 26 countries. J Clin Pathol. 2000; 53: 292-301.
(19) O'Leary, T. J. Standardization in immunohistochemistry. Appl Immunohistochem. Mol. Morph. 2001; 9: 3-8.
(20) Leong A S-Y. Quantitation in Immunohistology: Fact or Fiction? A Discussion of Variables That Influence Results. Appl Immunohist Mol. Morph. 2004; 12:1-7.
(21) Rhodes A, Jasani B, Anderson E et al. Evaluation of Her2/neu immunohistochemical assays sensitivity and scoring on formalin-fixed and paraffin-processed cell lines and breast tumors: a comparative study involving results from laboratories in 21 countries. Am J Clin Pathol. 2002; 118: 408-417.
(22) Bilous M, Dowsett M, Hanna W. et al. Current perspectives on HER 2 testing: A review of national testing guidelines. Mod Pathol. 2003; 16: 173-182.
(23) Hammond M E H, Barker P, Taube S et al. Standard Reference Material for Her2 Testing: Report of a National Institute of Standards and Technology-Sponsored Consensus Workshop. Appl Immunohist Mol. Morph. 2003; 11: 103-106.
(24) Shi S R, Cote R J, Taylor C R, Antigen Retrieval Immunohistochemistry: Past, Present and Future. J Histochem Cytochem 45:327-343, 1997.
(25) Shi S R, Gu J, Taylor C R. Antigen Retrieval Techniques: Immunohistochemistry and Molecular Morphology. Eaton Publishing, Natick, Mass. 2000.
(26) Shi S-R, Cote R J, and Taylor C R. Antigen Retrieval Immunohistochemistry and Molecular Morphology in the Year 2001. Appl Immunohist Mol Morph. 2001; 9:107-116.
(27) Nadji M, Nassiri M, Vinceck V et al. Immunohistochemistry of tissue prepared by a molecular friendly fixation and processing system. Appl Immunohistochem Mol. Morph. 2005: 13: 277-283.
(28) Taylor C. R. and Levenson R. M. Quantification of immunohistochemistry—issues around methods, utility and semi-quantitative assessment. Histopathology. 2006 (in press).
(29) Zu, Y., Steinberg, S. M., Campo, E., Hans, C. P., Weisenburger, D. D., Braziel, R. M., Delabie, J., Gascoyne, R. D., Muller-Hermlink, K., Pittaluga, S., Raffeld, M., Chan, W. C., and Jaffe, E. S. Validation of tissue microarray immunohistochemistry staining and interpretation in diffuse large B-cell lymphoma, Leukemia & Lymphoma. 2005: 46: 693-701.
(30) Riera, J., Simpson, J. F., Tamayo, R., and Battifora, H. Use of cultured cells as a control for quantitative immunocytochemical analysis of estrogen receptor in breast cancer. The Quicgel method, Am. J. Clin. Pathol. 1999; 111: 329-335.
(31) Sompuram, S. R., Kodela, V., Zhang, K., Ramanathan, H., Radcliffe, G., Falb, P., and Bogen, S. A. A novel quality control slide for quantitative immunohistochemistry testing, Journal of Histochemistry & Cytochemistry. 2002; 50: 1425-1434.
(32) Sompuram, S. R., Kodela, V., Ramanathan, H., Wescott, C., Radcliffe, G., and Bogen, S. A. Synthetic peptides identified from phage-displayed combinatorial libraries as immunodiagnostic assay surrogate quality-control targets. Clinical Chemistry. 2002; 48: 410420.
(33) Shi, S.-R., Liu, C., Perez, J., and Taylor, C. R. Protein-Embedding Technique: A Potential Approach to Standardization of Immunohistochemistry for Formalin-Fixed, Paraffin-Embedded Tissue Sections, J. Histochem. Cytochem. 2005; 53: 1167-1170.
(34) Shi, S.-R., Liu, C., Balgley, B. M., Lee, C., and Taylor, C. R. Protein extraction from formalin-fixed, paraffin-embedded tissue sections: quality evaluation by mass spectrometry, J. Histochem. Cytochem. JHC express. 2006; DOI:10.1369/jhc.5B6851.
(35) Taylor, C. R. and Burns, J. The demonstration of plasma cells and other immunoglobulin containing cells in formalin-fixed, paraffin-embedded tissues using peroxidase labeled antibody, J Clin Pathol. 1974; 27: 14-20.
(36) Battifora, H. Assessment of antigen damage in immunohistochemistry. The vimentin internal control, American Journal of Clinical Pathology. 1991; 96: 669-671.
(37) Capodieci, P., Donovan, M., Buchinsky, H., Jeffers, Y., Cordon-Cardo, C., W., G., J., E., M., S. S., and Singer, R. H. Gene expression profiling in single cells within tissue. Nature Methods. 2005; 2: 663-665.
(38) Shi, S. R., Cote, R. J., Yang, C., Chen, C., Xu, H. J., Benedict, W. F., and Taylor, C. R. Development of an optimal protocol for antigen retrieval: a ‘test battery’ approach exemplified with reference to the staining of retinoblastoma protein (pRB) in formalin fixed paraffin sections, J. Pathol. 1996; 179: 347-52.
(39) Willey, J. C., Crawford, E. L., Jackson, C. M., Weaver, D. A., Hoban, J. C., Khuder, S. A., and DeMuth, J. P. Expression measurement of many genes simultaneously by quantitative RT-PCR using standardized mixtures of competitive templates, Amer. J. Respiratory Cell Mol. Biol. 1998; 19: 16-24.
(40) Pagliarulo, V., George, B., Beil, S. J., Groshen, S., Laird, P. W., Cai, J., Willey, J., Cote, R. J., and Datar, R. H. Sensitivity and reproducibility of standardized-competitive RT-PCR for transcript quantification and its comparison with real time RT-PCR, Mol. Cancer. 2004; 3: 5.

### Example III

### IHC/ISH in Archival Tissues: Quantifiable Internal Reference Standards

One aspect of the present invention is that analytes (proteins and RNAs) that are present intrinsically within tissues may be employed as ‘Quantifiable Internal Reference Standards’, against which sample quality can be directly assessed and key analytes can be directly quantified⁽⁹⁴⁾.

In one embodiment of the present invention, a panel of candidate Quantifiable Internal Reference Standards (QIRS) has been assembled and tested based upon the measurement of proteins present in consistent amounts in common identifiable cells. A primary goal of this research is to demonstrate feasibility in establishing this panel. As described herein, Quantifiable Internal Reference Standards (QIRS) are an intrinsic part of the tissue, and by definition, have undergone identical sample preparation and IHC protocols to the test analyte, and thus serve both to validate sample preparation and also to calibrate the IHC stain, in effect converting the ‘stain’ it to an immunoassay for suited to quantification.

In connection with the present invention, quantification of a test analyte, such as an RNA transcripts in FFPE, is based on comparison to Quantifiable Internal Reference Standards (QIRS), and is reproducible from tissue to tissue, despite differences in fixation. In a parallel application of this method the highly variable degradation of RNA in sample preparation also has been evaluated by using internal standards intrinsic to the StaRT PCR method. This is in contrast to other methods of RNA analysis, which have focused on improved methods of extraction from FFPE, but do not measure degradation.

The All IHC immunoassays (stains for protein) of the present invention, are preferably in the form of ‘double IHC stain reactions’, including a ‘stain’ (IHC immunoassay) for a Quantifiable Internal Reference Standard (protein), and a second ‘stain’ (immunoassay) for the unknown ‘test’ analyte. The amount present of the unknown ‘test’ analyte (protein) may then be measured y comparison of the intensity of stain of the ‘test’ analyte with the intensity of stain of the Reference Standard, using validated quantitative IHC protocols and existing image analysis equipment and software.

In another embodiment of the present invention, a ‘correction factor’ and a ‘relative loss factor’ can be applied to provide a quantitative measurement of the amount of unknown test analyte present in the tissue prior to sample preparation (i.e., when it was removed from the patient).

A parallel rationale and method will develop quantitative ISH assays (stains) for RNA.

### Part I

The lack of reproducibility of immunohistochemical (and molecular) methods as applied to formalin fixed paraffin embedded (FFPE) tissue sections, or extracts thereof, constitutes a major obstacle to basic research, clinical trials and direct patient care. Our earlier work in this area⁽¹⁾ has led us to conclude, based on scientific and economic considerations, that

i. methods of sample preparation of tissues (including fixation) for surgical pathology will not be standardized in the next decade;

ii. universal external tissue reference standards also will not be available in the foreseeable future; and

iii. the scientific and patient care communities will therefore be forced to continue to work with FFPE tissues, in spite of manifold drawbacks. (ref Taylor and Becker 2011)

These conclusions apply to immunohistochemical (IHC) and in situ hybridization (ISH) methods applied to FFPE tissue sections, and to all analyses of proteins, RNA or DNA extracted from FFPE blocks. Furthermore, even if these problems could be solved, existing archival blocks would still not be addressable for quantitative analysis, and the numerous existing clinical trials (current and planned) that are dependent on data from archival FFPE materials would not be advantaged.

The QIRS methods of the present invention require neither standardized fixation nor external reference materials, and thus allows for quantitative assays on FFPE tissues. We have established a panel of candidate Quantifiable Internal Reference Standards in FFPE tissues, thereby serving two purposes simultaneously: (i) to control for the effects of variable sample preparation, and (ii) to provide the reference base for calibration and quantitative analysis of specific analytes.

The completed panels will be established and matched to suited ‘test analytes’ that show similar behavior in response to fixation and processing which will permit the established qualitative IHC ‘tissue stain’ to be converted into a quantifiable tissue based immunoassay for a wide range of molecules. QIRS IHC then becomes a widely applicable tissue based quantitative immunoassay, just like ELISA for analytes in serum or body fluids. Similarly existing qualitative ISH and FSH stains will be rendered quantitative. The following is a general description of the experimental design of studies performed to test the QIRS method, with specific aims for ongoing studies, that extend from proteins to the use of the QIRS for precise measurement of RNA and or DNA.

In order to qualify a test analyte as a QIRS, 2 analytes (each, for proteins and RNAs) will be selected as candidates for Quantifiable Internal Reference Standards, that are expected to be present at relatively constant concentrations within cell types that are common to (almost) all tissues, and it will be demonstrated that these selected proteins and RNAs are present during the steps of sample preparation (fixation/processing) in a consistent/predictable manner.

Preferably, extracts from the cell line blocks will be made at different steps of sample preparation and accurate measurements of the amount per cell of (a) each selected protein using standard ELISA methods, and (b) each selected mRNA using a standardized competitive RT-PCR (quantitative StaRT-PCR) will be made.

Preferably, a quantitative IHC methods will be constructed using the same antibody reagents as in the ELISA assays, and to validate IHC derived measurements of protein per cell by comparison to the ELISA data at each stage of sample preparation. This includes testing the IHC method for consistent generation of label (chromogen), to allow for strict quantification in cell block sections by image analysis methods.

Preferably, the range of protein and RNA analytes studied will be extended in order to construct a panel of 3 protein analytes (ideally one each of cytoplasmic, cell surface and nuclear proteins) and 3 RNA analytes as candidate internal reference standards in the FFPE cell line blocks.

To date 10 separate proteins have been assembled and subjected to preliminary testing by comparative IHC on tissues fixed for different times as candidate QIRS controls (6-30-10 supplement application)

These prototypic ‘internal reference’ panels for IHC, validated on FFPE cell line blocks have been extended application to normal human and porcine or swine tissues, and to human pathologic human FFPE tissues as TMAs (tissue micro arrays)

### Background and Significance

For reasons described herein, it is our belief that methods of sample preparation of tissues (including fixation) for surgical pathology will not be significantly improved (or standardized) in the next decade, and universal reference materials will not be available in the foreseeable future.

We have therefore developed an entirely novel approach, that utilizes FFPE tissues and does not require external reference materials, namely establishing Quantifiable Internal Reference Standards to address the major problem of non-reproducibility of IHC, ISH methods and to render them quantifiable.

From our ongoing experience of applying immunohistochemistry (IHC) and molecular methods to formalin fixed paraffin embedded (FFPE) tissues over 30 years⁽¹⁻⁷⁾, and our development of the Antigen Retrieval (AR) method over 15 years⁽⁸⁻¹³⁾, we believe that the impediments to achievement of reproducible IHC and ISH methods, that can yield quantitative results, fall into three areas:

1. lack of standardization of sample preparation (FFPE) within and across different laboratories, with variable and unknown degradation of both protein and RNA,

2. lack of reproducibility of AR, IHC and ISH methods within and across different laboratories,

3. failure to identify and establish universal reference materials (standards) for the major classes of analytes that allow calibration of the analytical method and quantification of the analyte.

These three problems clearly are interconnected. It is now generally accepted that attempts to standardize either, (1) methods of sample preparation, or (2) IHC/ISH staining protocols, are doomed to fail in the absence of widely available standard reference materials (3), that would allow absolute measurement of performance (including reproducibility) of the process as a whole.

Current approaches to improving the overall quality of IHC (and ISH) staining methods revolve around solving one or more of the three problems described above. RFA CA-07-015 addresses in particular problems 1 and 3—‘enhancement or adaptation of sample preparation methodologies—development of assays to assess sample quality’.

The rationale is sound, in that if these two problem areas (sample preparation and reference standards) are resolved then the solution to problem #2 should be relatively straightforward. However, we have concluded that there is no practically applicable solution in sight for these key problems.

Our work in this area, over many years, including our existing IMAT R33 award (Retrieval of DNA, RNA and Protein from Archival Tissues), has followed the conventional approaches outlined above. Significant advances have resulted from these efforts, including the first application of IHC to routine FFPE by the PI⁽²⁾, the development of Antigen retrieval (AR) methods for IHC by another of our group⁽⁸⁾, and the adaptation of AR for extracting proteins, RNA and DNA^{(14, 15)}. However, we ahave been forced to recognize that these conventional approaches, to improved sample preparation, antigen retrieval and reference standards, have failed to produce an overall system of IHC that assures quantitative results of uniform high quality, with reproducibility and reliability (Taylor Becker ref).

We have therefore concluded:

1. for sample preparation—that the scientific issues of developing a new fixative are challenging and not yet solved; more importantly the logistical and economic obstacles to replacing formalin, worldwide, with something better are formidable, such that there will not be an improved widely used sample preparation (fixation) procedure in the next decade.

2. for reproducibility of AR and IHC protocols—that current reagents and protocols are probably satisfactory, but further progress is dependent upon resolution to the problems of sample preparation and standard reference materials.

3. for reference standards—that the scientific issues of developing either FFPE ‘faux’ tissues or protein or RNA standards are significant, but again are dwarfed by the logistical and economic obstacles of manufacture, distribution and inclusion of any external reference standard into essentially all FFPE blocks going forward. Conventional ‘external’ reference standards for IHC or ISH on FFPE tissues will thus not become widely available in the foreseeable future.

These conclusions apply both to IHC and ISH on tissue sections and to all analyses of proteins, RNA or DNA extracted from FFPE blocks. Even if these problems could be solved, existing archival blocks would still not be addressable for quantitative analysis by any of these methods, and the numerous existing clinical trials that are dependent on data from archival FFPE materials would not be advantaged (Taylor Becker ref).

The focus of the QIRS approach is therefore radically different. It accepts the following as practical facts:
that we are going to be working with FFPE tissues for years to come,
that a universally available external reference standard for most IHC and ISH analytes will not become available the foreseeable future.

The QIRS approach emphasizes IHC methods, because IHC methods are currently widely used, and problematic in surgical pathology. However, ISH methods (for RNA or DNA) are included in parallel under the same QIRS approach with the belief that ISH would also be more widely used in if attendant problems of reproducibility and quantification could be resolved. Thus, while gene expression profiling has shown great promise in diagnosis, prognosis and therapy selection, the great impediment has been variable and unknown RNA degradation if FFPE tissues and extracts thereof.

Changing the Mindset from an IHC ‘Stain’ to an IHC ‘Analysis’

More then a decade ago the Biologic Stain Commission, in conjunction with the FDA, provided critical leadership in addressing the ‘standardization’ of IHC^{(1, 16, 17)}. Several conferences led to greatly improved standards for reagent validation package inserts⁽¹⁷⁻²⁸⁾. One contribution from our group was the recognition that an IHC stain could be more than just a simple stain; it should be viewed as an ‘in situ’ immunoassay in the tissue section environment, and should be managed in a manner identical to any other laboratory analysis. This led in turn to the formulation of the “Total Test Approach”^{(29, 30)}, borrowed directly from the rigorous and comprehensive test protocols used in quantitative assays in the clinical laboratory. In the ‘Total Test Approach’, all aspects of the assay are addressed; pre-analytic, analytic, and post-analytic, including interpreting and reporting of the results (Table 1), reviewed by the PI in Immunomicroscopy, A Diagnostic Tool for Surgical Pathologists⁽¹⁾.

TABLE 1
The Total Test: an IHC (or ISH) stain managed in the
same rigorous manner as a clinical laboratory analysis

| Pre-analytic | Analytic | Post analytic |
|---|---|---|
| Test selection | Antigen retrieval | Control performance |
| | procedure* | |
| Specimen type, | Protocol; control | Results |
| acquisition, transport | selection | |
| time* | | |
| Fixation, type and | Reagent validation | Interpretation/ |
| time* | | Reporting |
| Processing, | Technician | Pathologist, experience |
| temperature* | training/certification | and CME |
| | Laboratory certification | |

*Highly variable elements of ‘sample preparation’.

### Sample Preparation

One result of adopting the ‘Total Test Approach’ was to highlight the importance of specimen acquisition and sample preparation in contributing to the (lack of) quality of the end result of an IHC stain. In the Clinical Lab the response to a specimen that is incorrectly prepared (e.g., in the wrong anticoagulant, or outside of the specified transportation time), is that the test is rejected; not so in surgical pathology, where the general response is to embed the tissue and perform the stain, usually without even a notation of major variance in sample preparation. Where morphologic quality is the only arbiter of ‘adequate’ processing and handling (FFPE), the aforementioned response has sufficed for more than a hundred years, but today for IHC and ISH assays, it does not. This shortcoming has been recognized, albeit at subliminal level, for some time, with regard to the lack of reproducibility of the usual qualitative IHC, but little has been done about it, apart from recommendations from the BSC, CLSI (formerly NCCLS), UK-NEQAS and others^{(1, 17-19, 29-33)}. Now, however, as IHC and ISH methods are being employed in attempts to measure prognostic markers, the traditional cavalier approach to sample preparation (FFPE) has emerged as a critical problem. Now the question is “Just how much of the analyte (e.g., Her2) is present?” Not merely is it there, or not there, as might be sufficient in applying IHC to identify a lineage related marker (e.g. keratin in a putative ‘epithelial’ cell). The problem reached national attention with the increasing use of IHC findings, as entry criteria for patients into clinical trials (exemplified by staining for Her 2 or CD20, as indicators of possible effectiveness of monoclonal antibody therapy). The challenge became to ‘standardize’ the IHC or ISH stain (i.e., in effect, turn it into an assay), which in turn led to the recognition and then the affirmation that ‘sample preparation’ was a critical part of the process, and hence the issuance of the RFA CA 06-007 the essence of which is as follows ‘enhancement or adaptation of sample preparation methodologies and technologies—, the development of assays to assess sample quality’.

### Preliminary Studies:

Under our previous award (NIH 1 R33 CA103455-01-R21/R33 “Retrieval of DNA and RNA and Protein from Archival Tissues”) the possibilities of using AR derived methods for recovery and/or extraction of major classes of analytes from FFPE tissues have been extensively explored. Feasibility has been shown for qualitative demonstration of representative key analytes in tissue sections using Antigen Retrieval (AR) methods followed by IHC for protein, or ISH for RNA and DNA, using methods that are in general use in Pathology departments worldwide. Furthermore we have shown that extraction protocols derived from these same basic AR methods have been successful in recovery of proteins for Western blots and mass spectrometry analysis, and in recovery of DNA for Southern blots and PCR based methods^{(14, 15, 34)}. Dr Singer, an IMAT investigator and our consortium collaborator has shown initial successes for the demonstration of RNA in FFPE tissue sections⁽³⁵⁾.

As noted above, we have concluded that the scientific and practical problems fall into three major areas:

1. lack of standardization of sample preparation,

2. lack of reproducibility of AR and IHC (ISH) protocols,

3. lack of available universal reference materials (standards) for the major classes of analytes that would permit calibration of the analytical method and quantification.

Most approaches to improving the overall quality of IHC and ISH staining methods have revolved around solving one or more of the three problems described above. To date our approach has been different. We have recognized the intrinsic difficulties of achieving uniform improved sample preparation, and have instead used AR to ‘repair’ or ‘minimize’ the resultant variations.

### AR (‘Antigen Retrieval’) for IHC, ISH and Extraction of Analytes.

The problem of improved and standardized sample preparation (for FFPE), has not yet been solved. In addition, we recognize that solving the problem of ‘sample preparation’ going forward, still will not address the issue of performing studies on existing archival tissues, which form the basis for evaluating entry to current clinical trials. For these reasons we chose in our existing R33 proposal to focus upon the antigen retrieval (AR) approach, attempting to reverse the effects of formalin fixation, while possibly also minimizing the effects of varying fixatives and fixation times. In this regard the AR method has had major impact upon the application of IHC techniques to archival FFPE tissues, beginning in 1992, extending to today, when AR is in routine use in essentially all surgical pathology laboratories worldwide^{(1, 36-63)}. We have also reported success in adapting the basic AR methodology to extraction of proteins from FFPE sections for SDS-PAGE and mass spectrometric analysis and in extraction of DNA and RNA for PCR based analyses^{(14, 15, 34)}. However in the conduct of these studies we encountered significant limitations, namely that for all of these analyses, from IHC and ISH ‘stains’ in tissue sections, to mass spectrometry and PCR of tissue extracts, reproducibility remained poor and results that were qualitative rather than quantitative.

### Reference Standard—‘Faux’ Tissue and Protein Standards.

To begin to address the issues of reproducibility and quantification, we preferably explore the development of a universal reference standard. In this context we have reported the development of ‘faux’ tissues or histoids in collaboration with Drs. Imam and Ingram at the Huntington Research Institute^{(1, 64)}. The conclusions are that standardization of analyte (protein) content from batch to batch, while encouraging, is at present still unsatisfactory (the use of cell lines per se has of course been employed for a FDA approved Her2 ‘staining test’ (Dako), but the results are only crudely quantitative and are notoriously difficult to reproduce among labs and pathologists). Also the practical issues of ‘scale up’ to a level of production and development of methods of distribution that would make standardized histoids widely available, are at present insurmountable, primarily for economic reasons. We also have described prototypic work employing ‘protein embedded’ materials as a reference standard for defined antigens⁽⁶⁵⁾. The problems of developing purified protein standards, are both similar and different; similar in that the logistics of distributing any reference standard and incorporating the appropriate standard into FFPE blocks routinely (for each different stain) are daunting; different in that the technical challenges to preparing standard protein blocks that will survive FFPE, plus sectioning and staining have been explored by us, and others, with very limited success^{(1, 57, 65-71)}.

### Quantitative StaRT-PCR: Preliminary Data

While Validation Studies Performed to Date have Focused Upon IHC and Proteins, the Applicability of the Approach to RNA Quantification is Also Covered.

StaRT PCR, a standardized multi-gene expression analysis system that is an established technique in our laboratories⁽⁷⁸⁾. We have used it in developing the QIRS approach for application to RNA quantification. StaRT-PCR (Standardized Reverse Transcription Polymerase Chain Reaction developed by Gene Express Inc. Toledo, USA) offers a quantitative approach to measure gene expression and has been employed by us to generate data here at USC, and in collaboration with the Standardized Expression Measurement (SEM) Center at Toledo. The platform technique employs competitive templates incorporated into standardized mixtures of internal standards (SMIS) at precisely predetermined concentrations. These SMIS include internal standards for both the target and reference genes (e.g., ACTB). The data are represented as true numerical values that can be mathematically manipulated, allowing calculation of gene expression indices for the direct comparison of experimental results. Each gene expression result is reported as “number of molecules mRNA for gene per 10⁶ molecules of reference gene such as ACTB. Serial dilutions of the standardized mixes allow quantitative measurements over the 6 log range of gene expression. The StaRT PCR method will be made quantitative by use of ubiquitous or house-keeping RNAs as internal reference standards, such as beta actin or GAPDH (Table 4), and can compare transcript values numerically both within samples as well as across samples, providing a uniquely quantitative assay. The fixation and other preparatory steps of sample preparation leading to FFPE tissues will cause variable (and unknown) degradation of RNA. Our preliminary work leads us to believe that degradation is likely to affect different RNAs relatively uniformly, such that the internal reference standard RNA(s) and the test analyte RNAs will be affected similarly, allowing for quantification across different FFPE tissues, because the StaRT PCR quantification of target analyte depends upon comparison with the internal reference standard. Real time Q-RT-PCR is different; while it may be quantitative, it does not include this intrinsic control, and does not therefore lend itself to evaluating the different effects of degradation of different tissues.

Our proposal, which is entirely novel, is to combine the advantages of StaRT PCR with SMIS (standardized mixtures of internal standards), selecting the internal standards from within the FFPE tissues (i.e., QIRS or Quantifiable Internal reference Standards) in order to quantify RNA from tissue fixed under differing (unknown) conditions, such that starting copies of target (test) analytes are expressed relative to a known copy number (1,000,000) of the internal standard. Thus for this study the SMIS will in practice be the native templates within the FFPE tissues, that are subjected to exactly the same preparation steps as the test analyte, allowing quantification.

StaRT PCR is less than 10 years old has a technique, and has been little used. We have employed it in novel studies relating to clinical applicability and validation⁽⁷⁸⁾. We examined its applicability for molecular stage prediction in bladder cancer, employing both supervised and unsupervised data analysis through an iterative learning process called genetic programming. Transcript profiling data from bladder tumor tissue of 60 patients was examined by a N-fold cross validation technique for ‘genetic programming’, demonstrating 81% accuracy and 90% specificity in predicting nodal status. The StaRT PCR method proved to be reliable and reproducible in our hands, especially with respect to producing quantitative data⁽⁷⁸⁾.

RNA peT-FISH

This method for demonstrating gene expression profiles in individual cells in FFPE sections has been developed in the laboratory of our consortium collaborator, Dr. Singer⁽³⁵⁾, and was presented at the September 2005 IMAT meeting. The method was effective on a variety of FFPE tissues, yielding predictive quantitative gene expression signatures. This method provides the basis for development of a rigorously validated quantitative ISH method that will be intrinsic to this proposal.

### Need for a Different and Novel Approach

In accordance with the present invention, analytes (proteins and RNAs) present intrinsically within tissues and common to all (almost) tissue types may be employed as quantifiable internal reference standards, against which sample quality can be directly assessed and key analytes can be directly quantified.

Based upon our studies to date, in which QIRS and test antigens have been examined by simultaneous IHC dual or double stains, it is proposed that in the general application of the method all IHC assays (stains for protein), for which the goal is a quantifiable result, will in the future be in the form of ‘double IHC stain reactions’, including a ‘stain’ for a Quantifiable Internal Reference Standard, and a second ‘stain’ for the unknown ‘test’ analyte. The amount present of the unknown ‘test’ analyte (protein) may then be measured with accuracy (degree thereof to be established) by comparison of the intensity of stain of the ‘test’ analyte with the intensity of stain of the internal reference standard, using validated quantitative IHC protocols and existing image analysis equipment and software. Having previously established the extent to which the internal reference standard(s) is preserved following FFPE with optimized AR, then a ‘correction factor’) and a ‘relative loss factor’ can be applied to provide a quantitative measurement of the amount of unknown test analyte present in the tissue prior to the initiation of sample preparation (i.e., when it was removed from the patient The idea of utilizing ‘internal controls’ for simple qualitative assessment has wide prior use in traditional qualitative IHC, as exemplified by plasma cell staining in evaluating whether a stain for kappa chain has ‘worked’, or not (reviewed in⁽¹⁾). There is also precedent in the use of internal controls to assess crudely the extent of overall ‘loss of antigenicity’ following FFPE, by staining for vimentin, which is ‘formalin sensitive’ and is also present in almost all tissue samples⁽⁷²⁾; the implication being that the degree to which vimentin ‘stains’ may serve as an indicator of the expected degree of staining of other proteins (analytes). Also the idea that the effects of different FFPE processing during sample preparation may be minimized by the use of an optimized-AR protocol, resulting in improved reproducibility of IHC staining was pioneered by our group^{(1, 41, 66, 73)}, and has been proven effective for qualitative IHC studies among different laboratories^{(33, 74-76)}. There is also the important precedent in a prior IMAT sponsored study, of the work of Dr. Robert Singer, one of our collaborators, using house keeping gene RNAs (e.g., SMG mRNA, a gene expressed by all cells and detected in 40% of the cells in the tissue), as internal references standards the peT-FISH method applied to paraffin embedded tissues⁽³⁵⁾. Last there is the analogy of the standardized RT-PCR (StaRT PCR) method, which is quantitative by virtue of incorporation of standardized mixtures of internal standards (SMIS) at predetermined concentrations and comparison with internal actin mRNA transcripted (widely distributed in different cells) as the reference control⁽⁷⁷⁾. As described above we have successfully employed this technology to quantify transcripts in bladder cancer cell lines and tumor tissues, and demonstrated its superior reproducibility and consistency in relation to real time PCR⁽⁷⁸⁾. The quantitative character of StaRT PCR as applied to extracts y our laboratory make it the method of choice for independent validation of RNA degradation/recovery during sample preparation in establishing the FFPE FSIH quantifiable internal reference standards in this proposal.

### Overall Significance—Towards the Ultimate Goal of Molecular Morphology

In the year 2006, cancer still is diagnosed by the surgical pathologist with his/her microscope using methods that essentially are unchanged over 150 years, from the teaching of the first histology course (John Hughes Bennet, Edinburgh, 1842) to the first textbook of surgical pathology (Rudolph Virchow, Cellularpathologie, Berlin, 1858)^{(1, 94,95)}. That this remains true in 2006 is astonishing, in an era viewed by the public, politicians and many scientists, as the era of molecular biology and genetics. The primary reason for this anachronism is simple, that translation of ‘molecular methods’ from the bench to ‘routine’ diagnostic practice, has been greatly hindered by the fact that, worldwide, the method of sample preparation for surgical pathology is FFPE, which is satisfactory for the preservation of morphologic details, but is certainly not the method of choice for molecular immunologic assays (including ISH and IHC)⁽⁹⁴⁻⁹⁷⁾. The enormous variation in the actual protocols for FFPE employed in different labs, or in the same lab from specimen to specimen, compounds the problem and is a major factor in the current poor reproducibility of these methods. The availability of effective, reliable, quantitative IHC and ISH methods would allow visualization and ultra-cellular localization of key analytes, important to the diagnosis and prognosis of cancer, in conjunction with traditional surgical morphology criteria used for cell recognition and diagnosis. This combined dual capability is becoming known as Molecular Morphology. It is the raison d'etre of Applied Immunohistochemistry and Molecular Morphology), the journal of which the PI is the editor in chief. Molecular Morphology is in fact the basis of 80% of scientific papers published today in diagnostic surgical pathology. Surgical pathology (cancer diagnosis) has thus been totally transformed by the advent of IHC and AR methods to date⁽¹⁾. Rendering the method both reproducible and quantitative would mean that both IHC and ISH stains function as tissue based assays, not just stains, and that the future has arrived^{(94,95,97)}. Ultimately it will be possible reliably to measure RNA and protein, the end products of gene action, in situ within individual cells, leading to new criteria for cancer diagnosis and prognosis. In research the significance is equally profound, in that evaluation of gene activity (by RNA expression and protein production) allows scientists and clinicians to gain information at the molecular level regarding the function of genes. To be able to combine this capability with localization and quantification at a sub-cellular level will open new fields of study, particularly with regard to the pathogenesis of cancer.

### Research Design and Methods

The feasibility of using internal analytes as reference standards already has been shown for candidate protein QIRS. The construction and validation of quantitative IHC and ISH methods for wide spread application is thus intrinsic to the QIRS approach. Once established and tested with the corresponding reference standards, these methods will permit laboratories world wide to perform localization and measurement of a wide range of key analytes (proteins, RNAs and DNAs) within recognizable cell types in normal and pathologic tissues, combining the specificity of immunologic and molecular methods with morphologic criteria, for the diagnosis and prognosis of cancer, namely ‘molecular morphology’. In broadening the QIRS approach for gernal application the following steps are being pursued;

One aspect of the present invention is to select panels of analytes (of proteins and RNAs) as candidate Quantifiable Internal Reference Standards, that are expected to be present at relatively constant concentrations within cell types that are common to (almost) all tissues, and to demonstrate that these proteins and RNAs are present during the steps of sample preparation (fixation/processing) in a consistent/predictable manner.

The proteins for initial study were selected on the basis of our in house experience and the literature (e.g., CD45, CD20, vimentin, Her2)^{(1, 79-82)}. 6-30-10 supplementary data Other proteins were selected by preliminary IHC studies to confirm reported ranges of tissue distribution, (e.g., endothelial markers, CD31 and Fli1 widely distributed, CD34 and VWF variable⁽⁸³⁾), and to study the quality of available reagents (e.g., fibroblast surface protein using the Sigma IB10 antibody). RNA based studies will follow later, in parallel. RNA analytes (such as house keeping gene RNAs—see below) that are expected to be present at relatively constant concentrations within cell types that are common to (almost) all tissues, and to determine whether these RNAs are affected by the steps of sample preparation (fixation/processing) in a consistent/predictable manner. FFPE preparations (cell blocks) from cell lines have been used in some studies, but normal porcine or swine tissue proved more useful and more adaptable for fixation studies and TMAs. Cell line blocks yield pure cell populations for extraction of protein and RNA, and may in future validation prove more useful for quantification of test analytes resent in small amounts on normal tissues, or n restricted cell types Tissue sections with LCM methods may not yield sufficiently pure cell populations, and the cells that are obtained will not represent intact whole cells, having been cross cut in preparation of the section; they would not therefore be suited to calculating quantities of analyte on a per cell basis. Two to four cell lines will be selected as representative of four cell types commonly present in surgical pathology tissue sections; namely lymphocytes, endothelial cells, fibrocytes and epithelial cells (Table 4). These cell lines are all available in the USC laboratories and have been employed for the production of FFPE cell line blocks, by collecting aliquots of cells from culture, embedding in agar, fixing in 4% formaldehyde and then following ‘routine’ processing and paraffin embedment, with passage through xylene and graduated alcohols. In preliminary studies the selected cell lines will be grown in large batches and aliquots will be reserved for the different processing steps of FFPE. Fresh’ samples taken directly from active culture to liquid nitrogen will represent the ‘absolute’ reference standard for quantitative measurements. Other aliquots will be processed through the different steps of ‘routine sample preparation’ to FFPE pellet blocks as described above. Loss of analytes (protein or RNA) may be anticipated to occur at different steps in the sample preparation process, differing somewhat for proteins as a class, as opposed to RNA as a class (Table 2).

TABLE 2
Comparison of anticipated extent of loss/degradation of
proteins and RNA in sample preparation

| | Pre-fixation steps | Fixation/processing steps |
|---|---|---|
| Analytes | (degradation) | (‘formalin masking’) |
| Proteins | + to ++ | +++ to +++++ |
| RNA | +++++ | + to ++ |

(+, minor loss, to +++++, major loss)

In order to study these effects (losses of analyte) during the different steps of sample preparation different porcine cell blocks and cell line aliquots were subjected to differing ‘pre-fixation’ or hold periods (simulating time elapsed for removal of tissues from body and for transport to lab), with fixation time held constant, and to different fixation times, with the ‘pre-fixation’ (transport) step as time 0 (zero) minutes. The experimental construct is summarized in Table 3. Times will be adjusted to focus on ‘key areas of loss’ as preliminary results are obtained. The AR protocol to be employed is determined for each analyte by our published ‘test battery’ approach^{(41, 57, 73, 84, 85)} that has been widely adopted by research and service laboratories. This work was first performed on an exploratory panel of 10-12 ubiquitous proteins The initial proteins studied were from the cytoplasmic group, such as actin, vimentin, and B2 microglobulin, because of their ubiquity, relative abundance, established IHC staining protocols and reagents. A similar process will then be followed for RNA analytes. While exact correlations between the amount of protein and amount of RNA for any particular analyte are not expected, and losses may occur at differing steps in sample preparation, general trends may be observed for the corresponding analyte (e.g., Her2 protein and Her2 RNA) justifying the selection of protein/RNA pairings where ever feasible.

TABLE 3
Summary of representative study design for different protein/antibody
pairings.

| | | Pre-fix period | | | | | | | |
|---|---|---|---|---|---|---|---|---|---|
| | Absolute | (delays/ | | | | | | | AR - |
| Sample | fresh | transport, | | | FFPE | | | | Optimized |
| Prep'n | (unfixed) | etc.) | | | fixn time | | | | for each |
| Steps | min | mins | Hr | hrs | hrs | hrs | hrs | Hrs | analyte |
| Procedure | | | | | | | | | |
| for FFPE | 0 | 30 | 1 | 2 | 4 | 8 | 12 | 24 | AR + or − |
| section | | | | | | | | | |
| for extract | 0 | 30 | 1 | 2 | 4 | 8 | 12 | 24 | AR + or − |
| A. | | | | | | | | | |
| PROTEIN | | | | | | | | | |
| analytes | | | | | | | | | |
| FFPE | | | | | | | | | |
| section | | | | | | | | | |
| IHC | 0 | 30 | 1 | 2 | 4 | 8 | 12 | 24 | AR + or − |
| Extract | | | | | | | | | |
| ELISA | 0 | 30 | 1 | 2 | 4 | 8 | 12 | 24 | AR + or − |
| B. RNA | | | | | | | | | |
| analytes | | | | | | | | | |
| FFPE | | | | | | | | | |
| section | | | | | | | | | |
| PeT- | 0 min | 30 | 1 | 2 | 4 | 8 | 12 | 24 | AR + or − |
| FISH* | | | | | | | | | |
| Extract | | | | | | | | | |
| StaRT- | 0 min | 30 | 1 | 2 | 4 | 8 | 12 | 24 | AR + or − |
| PCR | | | | | | | | | |

*Tissues as much as 22 years old were used in pilot studies

Another aspect of the invention is: having qualified a number of candidate QIRS proteins by semi-quantitative IHC measurement of actual protein present by independent methods will be accomplished as described by Taylor C R, Becker K F. Liquid Morphology: Immunochemical Analysis of Proteins extracted from Formalin Fixed Paraffin Embedded Tissues: combining Proteomics with Immunohistochemistry, Appl. Immunohistochem & Mol Morphol, 19: 1-9: 2011, and summarized below, to make extracts from the tissue blocks or cell line blocks at different steps of sample preparation and measure accurately the amount per cell of (a) each selected protein using standard ELISA methods, and (b) each selected RNA using quantitative Start PCR.

(a) Protein. ELISA methods (enzyme linked immuno-sorbent assays) comprise one of the ‘standard methods’ for accurate measurement of proteins in serum in clinical laboratories, including our own clinical laboratories here at USC. The accuracy of ELISA is well established, with quantitative results derived by densitometric/colorimetric measurement of the unknown test analyte sample against a reference calibration curve generated from known (reference) standards (of the purified protein analyte) under strict protocol conditions. In this proposal, ELISA will be developed and performed to quantify the selected analytes in the ‘Extract’ aliquots, reflective of the different steps of sample preparation (Table 3). The ELISA assay will be established with the same reagents (primary antibodies) as are employed for the IHC stain protocols (see below), and the methods will be cross validated. By use of extracts of cell line preparations containing known numbers of cells, the ‘average’ amount of the reference analyte in an individual cell will be determined by the ELISA assay, and will then be used to calibrate the IHC method for amount analyte in a single cell as determined by quantitative image analysis. It is believed that the calibration of the IHC method versus ELISA can be established even in the event that FFPE processing renders protein extraction difficult, because calibration can also occur using the non-fixed materials. In addition, we believe that we will extract sufficient immunologically intact protein for ELISA studies, based on our experience in our existing R33 study (Retrieval of DNA, RNA and Protein from Archival Tissues), where this approach has in fact yielded sufficient amounts of intact protein for SDS PAGE analysis and for mass spectrometry, both in our laboratory and in collaboration with Calibrant, using their mass spectrometry system. ELISA also will be compared with calibrated Western blot gel methods⁽³⁴⁾; if the latter are more accurate and more cost effective then this approach may replace ELISA where possible. In addition to the above studies, Reverse Phase Protein Array (RPPA) developed by Becker and colleagues was shown to have distinct advantages for the purposed described herein, as reported by Taylor and Becker (2011)

(b) RNA. The same FFPE blocks will be used as for protein studies. Extracts of RNA will be made from cell line blocks using modified AR methods developed for recovery of analytes from archival tissues. The amount per cell of each selected mRNA will be measured using StaRT PCR, a standardized multi-gene expression analysis system that is an established technique in our laboratories⁽⁷⁸⁾. The StaRT PCR method will be made quantitative by use of ubiquitous or house-keeping RNAs as quantifiable internal reference standards (QIRS) as described. We will employ specific transcripts (e.g., actin, Table 4) as targets for StaRT PCR amplification in order to establish internal quantifiable standards; the transcript numbers will be expressed per million actin mRNA molecules. We will also investigate the use of beta-2-microglobulin and GAPDH transcripts as internal housekeeping gene quantifiers besides actin. Effects of variations in pre-fixation periods, nature of fixatives, and presence or absence of antigen retrieval procedures on the quantitative presence of the analytes will be assessed. The PCR method will be adapted for FFPE cell line blocks by use of competitive templates and target amplicons that are shorter than usual. This is because some degree of RNA degradation is expected during FFPE and the analytic method must address this degradation. We have found that the design and use of short competitive templates is straightforward, which makes the method uniquely amenable to the assay of partially degraded mRNA templates.

We recognize that StaRT PCR method was first published almost a decade ago, but our work is the first time that it has been adapted to extracts of FFPE sections. StaRT PCR is being used here as an independent measure of RNA degradation and recovery (for comparison with ISH data), in parallel to the use of ELISA to measure protein (for comparison with IHC). We have chosen to use StaRT PCR to measure RNA during the steps of sample preparation because intrinsic to the method is the use of internal controls, which allows assessment of variability of RNA degradation from FFPE block to FFPE block. Real time PCR has of course been used to quantify RNA in extracts of FFPE tissue, but it does not allow direct comparison of quantitative data from block to block and therefore does not allow for assessment of RNA degradation during sample preparation, a factor which is key to the current proposal. In addition, we have direct experience in quantitative and comparative use of the StaRT PCR method in our laboratory⁽⁷⁸⁾.

### Start PCR-Concise Method^{(77, 78)}

FFPE tissue sections will be lysed in TRIzol®, 400 μL of chloroform is then added, followed by centrifugation to separate the RNA-containing aqueous phase. Following addition of linear acrylamide (Ambion, Austin, Tex., USA) as a carrier and 1 mL of isopropanol to precipitate RNA, incubation at −80° C. for two hours, washing in cold 70% ethanol, and drying the RNA is resuspended in DEPC-treated water, for DNase treatment using DNA-free™ (Ambion, Austin, Tex., USA). cDNA is prepared using Superscript II as prescribed by the manufacturer (Invitrogen, Carlsbad, Calif., USA). Internal standard competitive template (CT) mixtures over 6 logs of concentration (A-F) will be obtained from Gene Express, Inc. (Toledo, Ohio). Each of the six mixtures contains internal standard CTs for nearly 400 target genes; our study will target a list of specific up to 6 transcripts (beginning with Table 4). Thus each sample will undergo six separate PCR analyses; each separate reaction containing the ready-to-use master mixture, cDNA sufficient for expression measurements of the target transcripts, primers for the target transcripts and one of the six CT mixes (including β-actin CT at a fixed concentration of 10⁻¹² M). The competitive PCR products will be electrophoresed using capillary electrophoresis in collaboration with Gene Express Inc. and image analysis and quantification of band fluorescence intensities will be done as prescribed by GeneExpress Inc. An aspect of the invention may be considered complete with the successful measurement of the average analyte per cell for 2 or more candidate reference proteins and 2 or more RNAs in 2 or more different cell line blocks at different stages of sample preparation as delineated in Table 3.

In a preferred embodiment, quantitative IHC methods are constructed, using the same antibody reagents as in the ELISA and Reverse Phase Protein Array (RPPA) assays. RPPA data have already been generated as described by Taylor and Backer (2011). This includes testing the IHC method for consistent generation of label (chromogen), to allow for strict quantification in cell block sections.

### IHC Staining Protocols and Reagents; Validation and Calibration to ELISA Methods

IHC methods as applied to tissue sections are strictly analogous to existing ELISA methods and will be constructed using the same reagents (primary antibodies) as are employed for the ELISA assay protocols. Using the QIRS approach the IHC method can calibrated for the amount analyte in a single cell as compared to the single cell average measured by ELISA, Quantitative image analysis is employed to ‘read’ the IHC staining results, using image analysis software and hardware such as the FDA approved Clarient/ChromaVision image analysis system will be used, with the addition of Spectral Analysis. The human eye cannot do this. Tests have been conducted on multiple replicate cell block FFPE sections to assure reproducibility of the IHC staining result (run to run, and batch to batch), and this approach will continue for QIRS-analyte pairings developed according to the present invention. In the event that consistent label generation proves difficult, immunogold methods may be employed, with a known and fixed average particle number per antibody molecule⁽⁸⁶⁻⁸⁸⁾. The IHC single and double stain methods have been used directly using the basic ABC method with peroxidase/DAB and alkaline phosphatase/fast red, performed on automated immunostainers with an open software program that allow for specifically tailored protocols to incorporate directly reagents identical to those used in the ELISA protocol. Mixed polymer based labels (from Biocare Medical) have also employed for double IHC methods, because of their excellent reproducibility in our hands, coupled with clear signals that have shown good results by differential spectral analysis proposed for the R33 phase. All of these methods are described in more detail by reference to the standard text—‘Immunomicroscopy”. A Diagnostic Tool for the Surgical Pathologist’ (Edited by the PI—Chapter 1)⁽¹⁾.

Extension of these initial data to a broad range of proteins and RNAs and translation into general laboratory use will require development of panels for different protein RNA groups as described in the following model system base dupon our development work to this point. The goal is to construct a panel of 3 protein analytes (ideally one each of cytoplasmic, cell surface and nuclear proteins) and 3 RNA analytes as candidate internal reference standards in the FFPE cell line blocks. The goal of assembling a ‘panel’ is to maximize the chances of finding a standard with similar characteristics (after FFPE) to clinically important test analytes All promising candidate standards from porcine tissues are being carried forward for testing on human tissues In the case of proteins those analytes identified as having consistent and predictable patterns of behavior during sample preparation, are considered as candidate reference standards. Additional cytoplasmic proteins, and then cell surface and nuclear proteins have been examined by ELISA, RPPA and IHC on FFPE ‘extracts’ and in ‘sections’ in an identical fashion (See Taylor Becker (2011) (Tables 3 and 4), again with the immediate goal of determining whether each or any of these additional analytes also show patterns of loss and recovery, after sample preparation and AR, that are consistent from block to block, and may apply to extensive groupings of test analytes (protein families) Analysis of the measured amount of ‘analyte per cell’ from the ELISA, RPPA, and IHC studies for aliquots of the sample at different steps of sample preparation (Table 3) provides the necessary data set to determine whether any of the tested proteins show a reproducible and predictable pattern of loss or retention under different conditions, such that correction factors can be derived to allow for accurate calculation of the amount of the protein in the original fresh cell line preparation. Parallel studies will be conducted for candidate RNA analytes by StaRT PCR on extracts (USC) and peT-FISH on FFPE sections (AECOM by Dr. Singer) to construct a RNA reference panel.

It is recognized that statistical treatment of the data and experimental design will be necessary to assure significance and validity of the findings on human tissues, once initial feasibility is established in cell line block studies; this design and work is reserved to the Part II phase. In addition as the work proceeds, if either of the protein of RNA methods show greater facility for the development of reference standard panels, then this aspect of the study will be advanced with the goal of testing human tissues at the earliest valid opportunity. Another aspect of the invention preferably includes 2 panels, one consisting of 3 (or more) reference proteins and another consisting of 3 (or more) reference RNAs, are assembled and tested in cell line blocks, by both IHC and ISH, according to the overall schematic shown in Table 4, recognizing that as the work proceeds it may be necessary to explore additional analytes, than those named. These will be selected for clinical utility and based upon initial findings as to which classes of proteins and RNAs show most promise after preliminary studies.

TABLE 4
Internal reference standards: candidate cell types and analytes having broad
tissue distribution. Selected data shown in supplental application Jun. 30, 2010

| | | | | Epithelial (breast) |
|---|---|---|---|---|
| Cell type | Lymphocyte (Raji | Endothelial cell | Fibroblast | (MCF7, |
| (Cell lines*) | or HL60) | (HuVEC) | (LD419) | MDA, MB468) |
| Analytes | | | | |
| Proteins | | | | |
| Cell Surface | CD45 | CD31 | Fibroblast | Her2 |
| | CD20 | | “surface protein” | EGFR |
| Cytoplasm | Actin | Actin | Actin | Actin |
| | B2 | B2 | B2 | B2 |
| | microglobulin | microglobulin | microglobulin | microglobulin |
| | Vimentin | Vimentin | Vimentin | Vimentin |
| | | Factor VIII | Factor VIII | |
| | | | Desmin | |
| Nucleus | Histone H1 | Histone H1 | Histone H1 | Histone H1 |
| | MiB1 (Ki-67) | MiB1 (Ki-67) | MiB1 (Ki-67) | MiB1 (Ki-67) |
| RNAs | | | | |
| Cell Surface | CD45 | CD31 | Fibroblast | Her2 |
| | CD20 | | “surface | EGFR |
| | | | protein” | |
| Cytoplasm | Actin | Actin | Actin | Actin |
| | B2 | B2 | B2 | B2 |
| | microglobulin | microglobulin | microglobulin | microglobulin |
| | Vimentin | Vimentin | Vimentin | Vimentin |
| | | Factor VIII | Desmin | |
| Nucleus | Histone H1 | Histone H1 | Histone H1 | Histone H1 |
| | SMG1 | SMG1 | SMG1 | SMG1 |

*All the cell lines listed are available in active growth at the KSOM Department of Pathology, either in the PI's laboratory or in collaboration with Dr. Alan Epstein, whose laboratory is located on the adjacent floor.

### Part II

Extension of QIRS to a Wide Range of Laboratory Analytes of Diagnostic Interest:

One can determine, using the same tissue samples, human or porcine, or cell line blocks that have been used to develop prototypic panels of ‘reference’ analytes (QIRS) (one for proteins, one for RNAs), once identified and quantified, can serve in a consistent predictive manner for other analytes, the QIRS being selected on the basis of being present only in some normal and pathologic tissues (i.e., does the quantified % loss of the reference analyte(s) have any predictive relationship to the % loss of other analytes [of similar class]—‘relative loss factor’)?

The quantitative peT-FISH method will be converted to a chromogenic label system, (CISH—chromogenic ISH), compatible with orthodox light microscopy on FFPE sections

One aspect of the present invention is to duplicate and extend using selected normal human tissue, the study design that was employed for protein on FFPE porcine tissues and cell blocks' (Table 5A), in order to establish reference panels for proteins in the ‘routine’ FFPE tissue section environment.

It is also contemplated to duplicate and extend using selected normal human tissue, the study design that was employed for RNA on FFPE cell blocks' (Table 5B), in order to establish the validity and utility of the reference panels for RNAs (developed for FFPE cell blocks) in the FFPE tissue section environment.

The QIRS to date have been tested on normal porcine or human tissue, not pathologic tissues. One aspect of the present invention to examine abnormal pathologic tissues, using the panels of internal reference standards established for protein in FFPE cell line blocks and FFPE normal human tissue and to test for the ability to quantify protein analytes by calculation of the amount of analyte per cell using correction and relative loss

In another embodiment of the present invention, double IHC stains have been employed, to allow comparison of the stain reaction for the reference analyte per cell) with the staining reaction for the test analyte (per cell), using quantitative spectral analysis. This method will include computer assisted algorithms for comparison and measurement, 510K approval will be pursued with the FDA based upon comparison with existing “gold standard methods,” which are less accurate,

In another embodiment of the present invention, abnormal pathologic tissues are examined, using the panels of internal reference standards established for RNA in FFPE cell line blocks and FFPE normal human tissue and to test for the ability to quantify protein analytes by calculation of the amount of analyte per cell using correction and relative loss factors

### Background and Significance:

QIRS when widely applied can provide the basis for establishing Molecular Morphology (the combination of quantitative molecular and morphologic criteria) as the method for cancer diagnosis, prognosis and therapy selection^{(94,95)}.

The ability to construct a panel of Quantifiable Internal Reference Standards, employing protein (and/or RNA) analytes that have a wide distribution in human tissues, and that have predictable behavioral characteristics when undergoing sample preparation (FFPE) is critical in providing the universal reference standards that these methodologies hitherto have lacked. Demonstrating the ability to construct panels of internal reference standards that can be applied to with IHC or ISH methods to measure accurately those analytes that do require accurate quantification has enormous significance, greatly advancing the discovery and use of prognostic markers. One application of the present invention (based on the assumption that an internal reference protein (e.g., vimentin) is (1) consistently detectable after FFPE at a level of, say, 50-60% of the amount originally present (in cells of fresh tissue) (i.e., has a stable correction factor), and (2) has a consistent relationship following FFPE and AR with a second (test) protein (e.g., Rb protein) (i.e., has a stable relative loss factor) is as follows: in a controlled double IHC stain the intensity of stain per cell for vimentin by comparison with the intensity of stain per cell for Rb protein, could be used to calculate the amount of vimentin per cell present prior to fixation (by use of the ‘correction factor’), as well as the amount of Rb present by calculation (the ‘relative loss factor’). On this basis it would then be possible to seek internal reference standards for key analytes, where quantification is critical. Again, by specific example, in order to develop an internal reference standard for, say, Her 2, an experimental search could be instituted for a ubiquitous protein that has a ‘relative loss factor’ in comparison with Her 2 protein that is consistent, and in addition has a stable ‘correction factor’ for sample preparation; double IHC staining of a FFPE section for Her 2 and the ‘standard’ would then allow accurate calculation of the amount of Her 2 present, using this internal control method, obviating therefore the errors contingent upon different methods of sample preparation. Distinction between two or more chromogens (or labels) will be needed, as will corrections for variations in section thickness and cell cuts across the section. It is envisaged that these steps will be accomplished by image analysis methods, including spectral imaging, which will be used to measure the intensity of stain of the reference standard on a mean cell basis, as the calibration marker for comparison with the intensity of stain of the test analyte.

It is emphasized that panels of Quantifiable Internal Reference Standards (QIRS) differ from ‘external standards’ (either proteins or cell lines) in the following important ways: 1. QIRS provide quality control of all steps of sample preparation, including ischemia time, fixation and processing steps; 2. QIRS provide a calibration standard for true quantitative assays; 3. QIRS, because they are intrinsic to the tissue section being ‘stained’, are inexhaustible, inexpensive and are universal, being automatically available for every IHC and ISH assay (stain). Quantifiable Internal Reference Standards thus meet all the requirements for a practical system of standards for IHC and ISH on FFPE sections that were developed at the 2002 NIST conference.⁽⁹⁴⁻⁹⁷⁾.

### Research Design and Methods—Future Studies for Validation and Extension of Application:

The protein and RNA panels developed in accordance with the present invention may extended to normal and pathologic human tissues as reference standards for a range of protein and RNA analytes by quantitative IHC and ISH methods.

One aspect of the presentation is to determine using the same cell blocks as in the R21 phase whether the 2 prototypic panels of ‘reference’ standards (one for proteins, one for RNAs), once identified and quantified, can serve in a consistent predictive manner for other analytes that are present in normal and pathologic tissues, i.e., does the quantified % loss of the reference standards(s) have any predictive relationship to the % loss of other analytes [of similar class]—‘relative loss factor’?

The answer to this question will determine whether one (or more) of the proteins (and/or RNAs) in these initial panels can serve as an internal reference standard, to assess the impact of sample preparation methods upon a broad range of proteins (antigens) (or RNAs) and to permit accurate quantification of such.

It is known that not all proteins behave in identical fashion during FFPE, so called formalin ‘sensitive’, ‘non-sensitive’ etc^{(66, 92)}. These classes of proteins show differing degrees of ‘loss/recovery’ after FFPE and AR; the goal of this study is to determine whether such ‘loss/recovery’ for a candidate reference protein analyte has consistency following sample preparation, such that the amount of analyte remaining in FFPE blocks after AR shows an acceptably consistent relationship to the amount originally present in the unfixed cell; as described previously; if such a consistent relationship can be demonstrated experimentally then this relationship can be calculated and codified as the ‘correction factor’ for that reference analyte. The correction factor can then be applied to the IHC stain reaction observed in FFPE cells (using image analysis) to calculate the amount of the reference analyte present in the unfixed state.

Each of the candidate reference analytes will be compared with each of the others in FFPE human tissues to determine whether there is a consistent relationship of each one, with any of the other reference standards thus far explored. For simplicity the experimentally determined relationship between a reference analyte and any other (test) analyte is herein termed the ‘relative loss factor’, and is a coefficient that codifies the effect of FFPE/AR on any one test protein as it relates to the effect of FFPE/AR on a selected reference standard that shows similar behavior during FFPE. It is intended that the test analytes (proteins and RNAs) selected and will be chosen from those with clinical relevance in surgical pathology diagnosis. With protein analytes these could include PSA, p53, Rb, estrogen receptor, again selected on the basis of current diagnostic utility. Her 2 would be included here if not already evaluated. Also it is recognized that ‘non-ubiquitous’ analytes will include a large number of ‘mutant’ proteins that are the product of gene mutations or translocations common in cancer cells, as well as novel RNA expression products. It is proposed that ‘relative loss factors’ may also be established by experimental demonstration for many of these proteins, and their corresponding RNAs. Data from our earlier published AR studies^{(73, 93)} suggest that the variety of responses of proteins to FFPE and the degree of recovery by AR is limited, and may allow most proteins of interest to be segregated into a small number of classes with regard to their behavior under these conditions. Such groupings might include, for example, formalin non-sensitive (<10% ‘loss’ after FFPE without AR), or formalin sensitive (with optimal AR at low pH, or mid-range pH, or high pH), or formalin sensitive with no useful recovery after AR. The exact categories are to be determined by experiment using data from the study, with the goal to identify and include in the panel at least one internal reference analyte from each category, which then would serve as the internal reference standard for other proteins in that category (also determined by experiment). By measurement of the intensity of IHC stain of the reference standard and comparison with the intensity of stain of the test analyte, and applying the derived ‘correction factor’ and ‘relative loss factor’ it would be possible to reach a calculated quantitative result. While absolute accuracy is not envisaged, it appears highly probable that results can be achieved that are far superior to current so called quantitative IHC measurements, that make no attempt to control for vagaries in sample preparations, and lack any objective reference standard whatsoever.

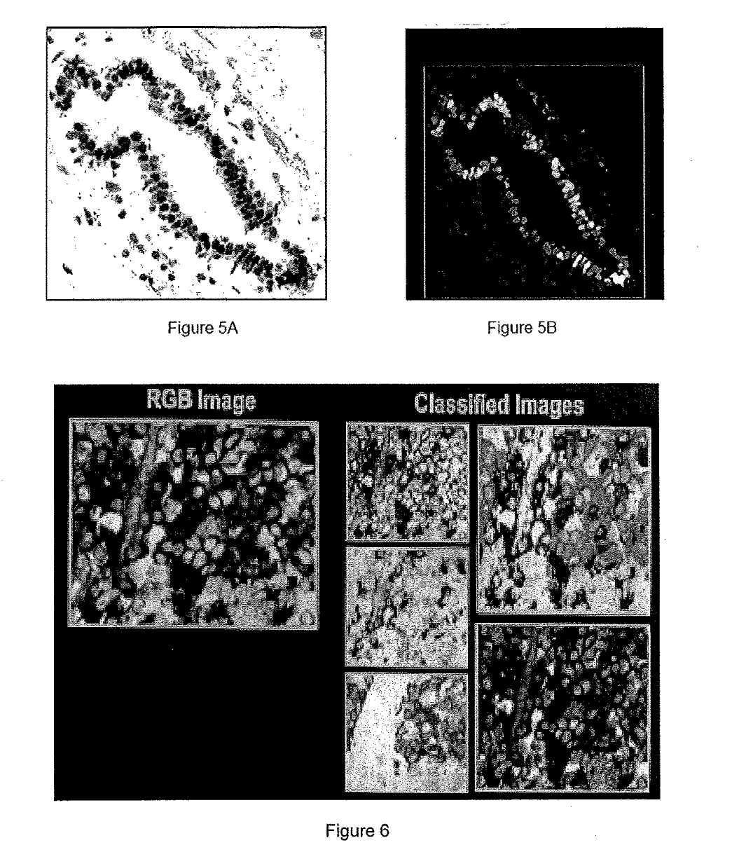

The measurement of the intensity of staining reaction of the reference standard in comparison to the test analytes in a double IHC stain will be performed using the Clarient system, but will be supplemented by spectral analysis using the Nuance Instrument and software⁽⁹⁵⁾. It is expected that this latter system (or others with like capabilities) will become the preferred approach because of accuracy and ease of application. The Nuance instrument and accompanying image analysis software allows for recognition, separation and measurement of different color signals (stains) and provides a means of quantifying any one against any other (see FIGS. 5-6).

Our existing data suggests that RNA, that is sufficiently intact for StaRT PCR can be extracted from FFPE tissues, while Dr. Singer has demonstrated that FISH methodology can be adapted successfully to demonstrate at least some RNA molecules in FFPE tissues. The patterns of loss (or recovery/retention) of RNA in FFPE are preferably consistent to a degree that allows for their use as general standards.

Preferably, a minimum of 3 ‘non-ubiquitous’ (test) proteins and 3 non-ubiquitous (test) RNAs, will be examined in comparison with the panel of internal reference standards, to determine whether consistent patterns and relationships exist, that allow accurate measurement by IHC (using correction and relative loss factors) of the amount of each analyte per cell, as compared to the corresponding ELISA and StaRT PCR measurements of the same analyte in the same cell population.

TABLE 5
Summary of study applied to normal and pathologic human tissue
blocks (tonsil)

| | | Pre-fix period | | | | | | | |
|---|---|---|---|---|---|---|---|---|---|
| | Absolute | (delays/ | | | | | | | AR - |
| Sample | fresh | transport, | | | FFPE | | | | Optimized |
| Prep'n | (unfixed) | etc.) | | | fixn time | | | | for each |
| Steps | min | Mins | hr | hrs | hrs | hrs | hrs | hrs | analyte |
| Procedure | | | | | | | | | |
| for FFPE | 0 | 30 | 1 | 2 | 4 | 8 | 12 | 24 | AR + or − |
| section | | | | | | | | | |
| for extract | 0 | 30 | 1 | 2 | 4 | 8 | 12 | 24 | AR + or − |
| A. | | | | | | | | | |
| PROTEIN | | | | | | | | | |
| analytes | | | | | | | | | |
| FFPE section | | | | | | | | | |
| IHC/Image | 0 | 30 | 1 | 2 | 4 | 8 | 12 | 24 | AR + or − |
| Analysis | | | | | | | | | |
| Extract | | | | | | | | | |
| ELISA | 0 | 30 | 1 | 2 | 4 | 8 | 12 | 24 | AR + or − |
| B. RNA | | | | | | | | | |
| analytes | | | | | | | | | |
| FFPE section | | | | | | | | | |
| peT- | 0 min | 30 | 1 | 2 | 4 | 8 | 12 | 24 | AR + or − |
| CISH/Image | | | | | | | | | |
| Analysis | | | | | | | | | |
| Extract* | | | | | | | | | |
| StaRT-PCR | 0 min | 30 | 1 | 2 | 4 | 8 | 12 | 24 | AR + or − |

*Parallels design for cell blocks - Table 3*

In a further embodiment, the peT-FISH method will be converted to a chromogenic label system compatible with orthodox light microscopy on FFPE sections—CISH (chromogenic ISH) which have been employed to demonstrate DNA amplification in FFPE sections; and to validate the selected method as described herein (or gold or silver label based method, as in GOLDFISH⁽⁹¹⁾ or SISH (silver ISH) if the chromogenic method does not lend itself to strict quantification).

The peT-FISH method will be adapted to a light microscopic environment that is compatible with detailed morphologic examination as in surgical pathology diagnosis, by replacing the fluorescent label with a stable chromogenic label (peT-CISH). If the chromogenic enzymatic label method does not allow strict quantification then we will move to labeling with gold particles (peT-GOLDFISH) or silver particles (peT-SISH). For these basic methodologies the reagents are widely available⁽¹⁾ and are already in use in our laboratory for research application in a non-quantitative manner. Our goal will be to adapt these qualitative methods to a rigorous quantitative assay, with validation for performed as described in Part I for the IHC method and for peT-FISH. The primary reason for converting the assay relates to its practical utility for surgical pathology, where light microscopy is the norm and immunofluorescence methods are employed only for limited applications, primarily because of incompatibility of the fluorescence method with evaluation of histologic criteria critical to the diagnosis. This modus operandi for surgical pathologist has not changed in 5 decades since immunofluorescence became available, and it is not going to change now. A second reason relates to the desire for a common ‘image analysis’ (hardware/software) approach to quantification, that is applicable both to IHC and ISH assays (stains), and will therefore be readily available to surgical pathologists. It is envisaged that automated assay protocols and computer assisted image analysis will be required for these quantitative methods. We believe that this outcome will be consistent with the new guidelines under development by the Clinical Lab Standards Institute (CLSI) and will likely be required by the FDA for approval of ‘quantitative’ IHC or ISH tests.

In a further embodiment, the methods of the present invention are extemded tp selected normal human tissue. in order to establish the validity and utility of the reference panels for proteins (developed for FFPE cell blocks) in the FFPE tissue section environment.

In a further embodiment, selected normal human tissue and the study design that was employed for RNA on FFPE cell blocks' will be extended to establish the validity and utility of the reference panels for RNAs (developed for FFPE cell blocks) in the FFPE tissue section environment.

Tonsil tissue may be used as the prototypic normal human tissue, because of the presence cell types that are candidates for the ‘ubiquitous’ cell types that would be expected to contain the reference analytes (lymphocytes, fibroblasts, endothelial cells and epithelial cells). Other candidate normal tissues include normal prostate, breast and spleen, that becomes routinely available in surgical pathology The analytes to be studied are listed in Table 4 in preliminary form, but may be change, addition or deletion, based upon the cell line block studies described.

In a further embodiment, abnormal pathologic tissues are examined, using the panels of internal reference standards established for protein in FFPE cell line blocks and FFPE normal human tissue, to test for the ability to quantify protein analytes by calculation of the amount of analyte per cell using correction and relative loss factors as described herein. Double IHC stains will be employed, to allow comparison of the stain reaction for the reference analyte (per cell) with the staining reaction for the test analyte (per cell), using quantitative spectral imaging and image analysis.

In a further embodiment, abnormal pathologic tissues will be examined, using the panels of internal reference standards established for RNA in FFPE cell line blocks and FFPE normal human tissue, to test for the ability to quantify protein analytes by calculation of the amount of analyte per cell using correction and relative loss factors as described herein. Double ISH stains will be employed, to allow comparison of the stain reaction for the reference analyte (per cell) with the staining reaction for the test analyte (per cell), using quantitative spectral imaging and image analysis. For this purpose the chromogenic/gold peT-ISH method developed as described herein will be employed.

The experimental design and methodology are analogous to the R21 embodiments (Tables 3 and 4), and the studies of normal human tissues described herein. With respect to pathologic tissues, additional challenges exist and there are additional questions to ask, and answer. It is anticipated that most pathologic tissues will contain common cell types (lymphocytes, fibroblasts, endothelial cells, often epithelial cells) that in turn express one or more of the reference standard analytes (proteins and RNA). It will be necessary to establish that these analytes are present and that their expression and behavior following FFPE is consistent (i.e., stable correction factor) so as to allow their use as internal standards. A larger challenge will be that many of the ‘test’ analytes will be uncommon in distribution, or even unique to particular tumor types, or to particular cells within the tumor. It will be necessary to determine, again experimentally, that consistent relationships exist between and test analyte (protein or RNA) and one or more of the established internal reference standards (i.e., stable relative loss factor). It should be emphasized that the investigators do recognize that the number of protein and RNA analytes that have been discovered, and will continue to be discovered, is very large, and that the scope of this grant is to establish the feasibility of this approach and to set up methods and protocols for determining the relevant correction and relative loss factors for new internal standards and for new test analytes.

The study may help to develop improved methods for cancer diagnosis and prognosis by means of standardized immunohistochemical and in situ hybridization methods applied to formalin paraffin sections.

### REFERENCES

⁽¹⁾Taylor, C. R. and Cote, R. J. Immunomicroscopy. A Diagnostic Tool for the Surgical Pathologist, 3rd edition. Philadelphia: Elsevier Saunders, 2005.
⁽²⁾Taylor, C. R. and Burns, J. The demonstration of plasma cells and other immunoglobulin containing cells in formalin-fixed, paraffin-embedded tissues using peroxidase labeled antibody, J. Clin. Pathol. 27: 14-20, 1974.
⁽³⁾Taylor, C. R. The nature of Reed-Sternberg cells and other malignant “reticulum” cells, Lancet. 2(7884): 802-807, 1974.
⁽⁴⁾Taylor, C. R. A history of the Reed-Sternberg cell, Biomedicine. 28: 196, 1978.
⁽⁵⁾Taylor, C. R. Immunohistologic studies of lymphomas: New methodology yields new information and poses new problems, J. Histochem. Cytochem. 27: 1189-1191, 1979.
⁽⁶⁾Taylor, C. R. Immunohistologic studies of lymphoma: Past, present and future. J. Histochem. Cytochem. 28: 777-787, 1980.
⁽⁷⁾Taylor, C. R. and Kledzik, G. Immunohistologic techniques in surgical pathology. A spectrum of new special stains, Hum. Pathol. 12: 590-596, 1981.
⁽⁸⁾Shi, S. R., Key, M. E., and Kalra, K. L. Antigen retrieval in formalin-fixed, paraffin-embedded tissues: an enhancement method for immunohistochemical staining based on microwave oven heating of tissue sections, J. Histochem. Cytochem. 39: 741-748, 1991.
⁽⁹⁾Shi, S. R., Cote, C., Kalra, K. L., Taylor, C. R., and Tandon, A. K. A technique for retrieving antigens in formalin-fixed, routinely acid-decalcified, celloidin-embedded human temporal bone sections for immunohistochemistry, J. Histochem. Cytochem. 40: 787-792, 1992.
⁽¹⁰⁾Shi, S. R., Chaiwun, B., Young, L., Cote, R. J., and Taylor, C. R. Antigen retrieval technique utilizing citrate buffer or urea solution for immunohistochemical demonstration of androgen receptor in formalin-fixed paraffin sections, J. Histochem. Cytochem. 41: 1599-1604, 1993.
⁽¹¹⁾Shi, S. R., Chaiwun, B., Young, L., Imam, A., Cote, R. J., and Taylor, C. R. Antigen retrieval using pH 3.5 glycine-HCl buffer or urea solution for immunohistochemical localization of Ki-67, Biotech. Histochem. 69: 213-215, 1994.
⁽¹²⁾Shi, S.-R., Gu, J., Kalra, K. L., Chen, T., Cote, R. J., and Taylor, C. R. Antigen retrieval technique: a novel approach to immunohistochemistry on routinely processed tissue sections, Cell Vision. 2: 6-22, 1995.
⁽¹³⁾Shi, S. R., Cote, R. J., Young, L., Imam, S. A., and Taylor, C, R. Use of pH 9.5 Tris-HCl buffer containing 5% urea for antigen retrieval immunohistochemistry, Biotech. Histochem. 71: 190-196, 1996.
⁽¹⁴⁾Shi, S.-R., Cote, R. J., Wu, L., Liu, C., Datar, R., Shi, Y., Liu, D., Lim, H., and Taylor, C. R. DNA extraction from archival formalin-fixed, paraffin-embedded tissue sections based on the antigen retrieval principle: heating under the influence of pH, J. Histochem. Cytochem. 50: 1005-1011, 2002.
⁽¹⁵⁾Shi, S.-R., Datar, R., Liu, C., Wu, L., Zhang, Z., Cote, R. J., and Taylor, C. R. DNA extraction from archival formalin-fixed, paraffin-embedded tissues: heat-induced retrieval in alkaline solution, Histochem. Cell Biol. 122: 211-218, 2004.
⁽¹⁶⁾Taylor, C. R. Report of the Immunohistochemistry Steering Committee of the Biologixal Stain Commission. “Proposed Format: Package Insert for Immunohistochemistry Products”, Biotech. Histochem. 67: 323-338, 1992.
⁽¹⁷⁾Taylor, C. R., Shi, S. R., Chaiwun, B., Young, L., Imam, S. A., and Cote, R. J. Strategies for improving the immunohistochemical staining of various intranuclear prognostic markers in formalin-paraffin sections: androgen receptor, estrogen receptor, progesterone receptor, p53 protein, proliferating cell nuclear antigen, and Ki-67 antigen revealed by antigen retrieval techniques [see comments], Hum. Pathol. 25: 263-270, 1994.
⁽¹⁸⁾Taylor, C. R. The current role of immunohistochemistry in diagnostic pathology, Advan. Pathol. Lab. Med. 7: 59-105, 1994.
⁽¹⁹⁾Taylor, C. R., Shi, S.-R., Chaiwun, B., Young, L., Imam, S. A., and Cote, R. J. Correspondence. Standardization and reproducibility in diagnostic immunohistochemistry, Hum. Pathol. 25: 1107-1109, 1994.
⁽²⁰⁾Battifora, H. Quality assurance issues in immunohistochemistry, J. Histotechnol. 22: 169-175, 1999.
⁽²¹⁾Bhan, A. K. Chapter 38, Immunoperoxidase, 2nd edition, p. 711-723. New York: Raven Press, 1995.
⁽²²⁾Colvin, R. B., Bhan, A. K., and McCluskey, R. T. Diagnostic Immunopathology, 2nd edition. New York: Raven Press, 1995,
⁽²³⁾Dapson, R. W. Fixation for the 1990's: a review of needs and accomplishments, Biotech. Histochem. 68: 75-82, 1993.
⁽²⁴⁾DeLellis, R. A., Sternberger, L. A., Mann, R. B., Banks, P. M., and Nakane, P. K. Immunoperoxidase techniques in diagnostic pathology. Report of a workshop sponsored by the National Cancer Institute, Am. J. Olin. Pathol. 71: 483-488, 1979.
⁽²⁵⁾DeLellis, R. A. Advances in Immunohistochemistry. p. 1-45. New York: Raven Press, 1988.
⁽²⁶⁾Elias, J. M. Immunohistopathology: A Practical Approach to Diagnosis, 1st edition, p. 1-9. Chicago: ASCP Press, 1990.
⁽²⁷⁾Elias, J. M. Commentary: Immunohistochemistry: a brief historical perspective. In: S.-R. Shi, J. Gu, and C. R. Taylor (eds.), Antigen Retrieval Techniques: Immunohistochemistry and Molecular Morphology, pp. 7-13, 2000.
⁽²⁸⁾Larsson, L.-I. Immunocytochemistry: Theory and Practice, p. 41-170. Boca Raton, Fla.: CRC Press, 1988.
⁽²⁹⁾Taylor, C. R. Quality assurance and standardization in immunohistochemistry. A proposal for the annual meeting of the Biological Stain Commission., Biotech. Histochem, 67:110-117, 1992.
⁽³⁰⁾Taylor, C. R. An exaltation of experts: concerted efforts in the standardization of immunohistochemistry, Hum. Pathol. 25: 2-11, 1994.
⁽³¹⁾Taylor, C. FDA issues final rule for classification of reclassification of immunochemistry reagents and kits, Am. J. Clin. Pathol. 111: 443-444, 1999.
⁽³²⁾Rhodes, A., Jasani, B., Balaton, A. J., and Miller, K. D. Immunohistochemical demonstration of oestrogen and progesterone receptors: correlation of standards achieved on in house tumours with that achieved on external quality assessment material in over 150 laboratories from 26 countries, J. Clin. Pathol. 53: 292-301, 2000.
⁽³³⁾Rhodes, A., Jasani, B., Balaton, A. J., Barnes, D. M., Anderson, E., Bobrow, L. G., and Miller, K. D. Study of interlaboratory reliability and reproducibility of estrogen and progesterone receptor assays in Europe: documentation of poor reliability and identification of insufficient microwave antigen retrieval time as a major contributory element of unreliable assays, Am. J. Clin. Pathol. 115: 44-58, 2001.
⁽³⁴⁾Shi, S.-R., Liu, C., Balgley, B. M., Lee, C., and Taylor, C. R. Protein extraction from formalin-fixed, paraffin-embedded tissue sections: quality evaluation by mass spectrometry, J. Histochem. Cytochem. JHC exPress, DOI:10.1369/jhc.5B6851.2006.
⁽³⁵⁾Capodieci, P., Donovan, M., Buchinsky, H., Jeffers, Y., Cordon-Cardo, C., W., G., J., E., M., S. S., and Singer, R. H. Gene expression profiling in single cells within tissue, Nature Methods. 2: 663-665, 2005.
⁽³⁶⁾Taylor, C. R., Shi, S.-R., and Cote, R. J. Antigen retrieval for immunohistochemistry. Status and need for greater standardization, Appl. Immunohistochem. 4: 144-166, 1996.
⁽³⁷⁾Shi, S. R., Cote, R. J., and Taylor, C. R. Antigen retrieval immunohistochemistry: past, present, and future, J. Histochem. Cytochem. 45: 327-343, 1997.
⁽³⁸⁾Shi, S.-R., Cote, R. J., Young, L. L., and Taylor, C. R. Antigen retrieval immunohistochemistry: practice and development, J. Histotechnol. 20: 145-154, 1997.
⁽³⁹⁾Shi, Y., Li, G.-D., and Liu, W.-P. Recent advances of the antigen retrieval technique, Linchuang yu Shiyan Binglixue Zazhi (J. Clin. Exp. Pathol.), 13: 265-267, 1997.
⁽⁴⁰⁾Shi, S. R., Cote, R. J., and Taylor, C. R. Antigen retrieval immunohistochemistry used for routinely processed celloidin-embedded human temporal bone sections: standardization and development, Auris Nasus Larynx, 25: 425-443, 1998.
⁽⁴¹⁾Shi, S.-R., Cote, R. J., and Taylor, C. R. Standardization and further development of antigen retrieval immunohistochemistry: strategies and future goals, J. Histotechnol. 22: 177-192, 1999.
⁽⁴²⁾Shi, Cote, R. J., Shi, Y., and Taylor, C. R. Antigen retrieval technique. In: S.-R. Shi, J. Gu, and C. R. Taylor (eds.), Antigen Retrieval Techniques: Immunohistochemistry and Molecular Morphology, 1st. edition. Natick, Mass.: Eaton Publishing, 2000.
⁽⁴³⁾Boon, M. E. and Kok, L. P. Breakthrough in pathology due to antigen retrieval, Mal. J. Med. Lab. Sci. 12: 1-9, 1995.
⁽⁴⁴⁾Brown, C. Antigen retrieval methods for immunohistochemistry, Toxicol. Pathol. 26: 830-831, 1998.
⁽⁴⁵⁾Cuevas, E. C., Bateman, A. C., Wilkins, B. S., Johnson, P. A., Williams, J. H., Lee, A. H., Jones, D. B., and Wright, D. H. Microwave antigen retrieval in immunocytochemistry: a study of 80 antibodies, J. Olin. Pathol. 47: 448-452, 1994.
⁽⁴⁶⁾Evers, P., Uylings, H. B., and Suurmeijer, A. J. Antigen retrieval in formaldehyde-fixed human brain tissue, Methods. 15: 133-140, 1998.
⁽⁴⁷⁾Gown, A. M., de Wever, N., and Battifora, H. Microwave-based antigenic unmasking. A revolutionary new technique for routine immunohistochemistry, Appl. Immunohistochem. 1: 256-266, 1993.
⁽⁴⁸⁾Gown, A. M. Unmasking the mysteries of antigen or epitope retrieval and formalin fixation, Am. J. Olin. Pathol. 121: 172-174, 2004.
⁽⁴⁹⁾Grabau, K. A., Nielsen, O., Hansen, S., Nielsen, M. M., Lankholm, A.-V., Knoop, A., and Pfeiffer, P. Influence of storage temperature and high-temperature antigen retrieval buffers on results of immunohistochemical staining in sections stored for long periods, Appl. Immunohistochem. 6: 209-213, 1998.
⁽⁵⁰⁾Hopwood, D. Epitope retrieval—survey and prospect, Eur. J. Morphol. 32: 317-324, 1994.
⁽⁵¹⁾Krenacs, L., Krenacs, T., and Raffeld, M. Antigen retrieval for immunohistochemical reactions in routinely processed paraffin sections, Methods Mol. Biol. 115: 85-93, 1999.
⁽⁵²⁾Leong, A. S. Microwaves in diagnostic immunohistochemistry, Eur. J. Morphol. 34: 381-383, 1996.
⁽⁵³⁾Leong, A. S.-Y., Lee, E. S., Yin, H., Kear, M., Haffajee, Z., and Pepperall, D. Superheating antigen retrieval, Appl. Immunohistochem. Mol. Morphol. (AIMM). 10: 263-268, 2002.
⁽⁵⁴⁾Miller, R. T. and Estran, C. Heat-induced epitope retrieval with a pressure cooker-suggestions for optimal use, Appl. Immunohistochem. 3: 190-193, 1995.
⁽⁵⁵⁾Miller, R. T., Swanson, P. E., and Wick, M. R. Fixation and epitope retrieval in diagnostic immunohistochemistry: a concise review with practical considerations, Appl. Immunohistochem. MeI. Morphol. (AIMM). 8: 228-235, 2000.
⁽⁵⁶⁾Newman, S. J. and Gentleman, S. M. Microwave antigen retrieval in formaldehyde-fixed human brain tissue, Methods Mol. Biol. 72: 145-152, 1997.
⁽⁵⁷⁾O'Leary, T. J. Standardization in immunohistochemistry, Appl. Immunohistochem. Mol. Morphol. (AIMM). 9: 3-8, 2001.
⁽⁵⁸⁾Pileri, S. A., Roncador, G., Ceccarelli, C., Piccioli, M., Briskomatis, A., Sabattini, E., Ascani, S., Santini, D., Piccaluga, P. P., Leone, O., Damiani, S., Ercolessi, C., Sandri, F., Pieri, F., Leoncini, L., and Falini, B. Antigen retrieval techniques in immunohistochemistry: comparison of different methods, J. Pathol. 183: 116-123, 1997.
⁽⁵⁹⁾Reynolds, G. M., Young, F. I., Young, J. A., Williams, A., and Rowlands, D. C. Microwave oven antigen retrieval applied to the immunostaining of cytopathology specimens, Cytopathology. 5: 345-358, 1994.
⁽⁶⁰⁾Rocken, C. and Roessner, A. An evaluation of antigen retrieval procedures for immunoelectron microscopic classification of amyloid deposits, J. Histochem. Cytochem. 47: 1385-1394, 1999.
⁽⁶¹⁾Stirling, J. W. and Graff, P. S. Antigen unmasking for immunoelectron microscopy: labeling is improved by treating with sodium ethoxide or sodium metaperiodate, then heating on retrieval medium, J. Histochem. Cytochem. 43: 115-123, 1995.
⁽⁶²⁾Swanson, P. E. Microwave antigen retrieval in citrate buffer, Lab. Med. 25: 520-522, 1994.
⁽⁶³⁾Werner, M., Von Wasielewski, R., and Komminoth, P. Antigen retrieval, signal amplification and intensification in immunohistochemistry, Histochem. Cell Biol. 105: 253-260, 1996.
⁽⁶⁴⁾Ingram, M., Techy, G. B., Saroufeem, R., Yazan, O., Narayan, K. S., Goodwin, T. J., and Spaulding, G. F. Three-dimensional growth patterns of various human tumor cell lines in simulated microgravity of a NASA bioreactor, In Vitro Cellular & Developmental Biology. Animal. 33: 459-466, 1997.
⁽⁶⁵⁾Shi, S.-R., Liu, C., Perez, J., and Taylor, C. R. Protein-Embedding Technique: A Potential Approach to Standardization of Immunohistochemistry for Formalin-Fixed, Paraffin-Embedded Tissue Sections, J. Histochem. Cytochem. 53: 1167-1170, 2005.
⁽⁶⁶⁾Shi, S.-R., Gu, J., Cote, R. J., and Taylor, C. R. Chapter 16. Standardization of routine immunohistochemistry: where to begin? In: S.-R. Shi, J. Gu, and C. R. Taylor (eds.), Antigen Retrieval Technique: Immunohistochemistry and Molecular Morphology, First edition, pp. 255-272. Natick, Mass.: Eaton Publishing, 2000.
⁽⁶⁷⁾Risio, M. Methodological aspects of using immunohistochemical cell proliferation biomarkers in colorectal carcinoma chemoprevention, J. Cell Biochem. Suppl. 19: 61-67, 1994.
⁽⁶⁸⁾Riera, J., Simpson, J. F., Tamayo, R., and Battifora, H. Use of cultured cells as a control for quantitative immunocytochemical analysis of estrogen receptor in breast cancer. The Quicgel method, Am. J. Olin. Pathol. 111: 329-335, 1999.
⁽⁶⁹⁾Mighell, A. J., Hume, W. J., and Robinson, P. A. An overview of the complexities and subtleties of immunohistochemistry, Oral Dis. 4: 217-23, 1998.
⁽⁷⁰⁾Sompuram, S. R., Kodela, V., Zhang, K., Ramanathan, H., Radcliffe, G., Falb, P., and Bogen, S. A. A novel quality control slide for quantitative immunohistochemistry testing, J. Histochem. Cytochem. 50: 1425-1434, 2002.
⁽⁷¹⁾Sompuram, S. R., Kodela, V., Ramanathan, H., Wescott, C., Radcliffe, G., and Bogen, S. A. Synthetic peptides identified from phage-displayed combinatorial libraries as immunodiagnostic assay surrogate quality-control targets, Olin. Chem. 48: 410-420, 2002.
⁽⁷²⁾Battifora, H. Assessment of antigen damage in immunohistochemistry. The vimentin internal control, Am. J. Clin. Pathol. 96: 669-671, 1991.
⁽⁷³⁾Shi, S.-R., Cote, R. J., Chaiwun, B., Young, L. L., Shi, Y., Hawes, D., T., C., and Taylor, C. R. Standardization of immunohistochemistry based on antigen retrieval technique for routine formalin-fixed tissue sections, Appl. Immunohistochem. 6: 89-96, 1998.
⁽⁷⁴⁾Rhodes, A., Jasani, B., Andersion, E., Dodson, A. R., and Balaton, A. J. Evaluation of HER-2/neu Immunohistochemical Assay Sensitivity and Scoring on Formalin-Fixed and Paraffin-Processed Cell Lines and Breast Tumors, Am. J. Olin. Pathol. 118: 408-417, 2002.
⁽⁷⁵⁾Jacobs, T. W., Gown, A. M., Yaziji, H., AM., B., and Schnitt, S. J. HER-2/neu Protein Expression in Breast Cancer Evaluated by Immunohistochemistry, Am. J. Clin. Pathol. 113: 251-258, 2000.
⁽⁷⁶⁾Lambkin, H. A., Dunne, P., and McCarthy, P. M. Standardization of estrogen-receptor analysis by immunohistochemistry—an assessment of interlaboratory performance in Ireland, Appl. Immunohistochem. 6: 103-107, 1998.
⁽⁷⁷⁾Willey, J. C., Crawford, E. L., Jackson, C. M., Weaver, D. A., Hoban, J. C., Khuder, S. A., and DeMuth, J. P. Expression measurement of many genes simultaneously by quantitative RT-PCR using standardized mixtures of competitive templates, Am. J. Res. Cell Mol. Biol. 19: 16-24, 1998.
^{(78a)}Pagliarulo, V., George, B., Beil, S. J., Groshen, S., Laird, P. W., Cai, J., Willey, J., Cote, R. J., and Datar, R. H. Sensitivity and reproducibility of standardized-competitive RT-PCR for transcript quantification and its comparison with real time RT-PCR, Mol. Cancer. 3: 5, 2004.
^{(78b)}Mitra A P, Almal A A, George B, Fry D W, Lenehan P F, Pagliarulo V, Cote, R J, Datar, R H, Worzel W P. The use of genetic programming in the analysis of quantitative gene expression profile for nodal status in bladder cancer. BMC Cancer. 6: 159, 2006.
⁽⁷⁹⁾Leong, A. S.-Y. Immunostaining of Cytologic Preparations: A Review of Technical Problems, Appl. Immunohistochem. Mol. Morphol. 7: 214-220, 1999.
⁽⁸⁰⁾Grizzle, W. E., Myers, R. B., and Oelschlager, D. K. Prognostic biomarkers in breast cancer: factors affecting immunohistochemical evaluation, Breast. 1: 243-250, 1995.
⁽⁸¹⁾Zu, Y., Steinberg, S. M., Campo, E., Hans, C. P., Weisenburger, D. D., Braziel, R. M., Delabie, J., Gascoyne, R. D., Muller-Hermlink, K., Pittaluga, S., Raffeld, M., Chan, W. C., and Jaffe, E. S. Validation of tissue microarray immunohistochemistry staining and interpretation in diffuse large B-cell lymphoma, Leukemia & Lymphoma. 46: 693-701, 2005.
⁽⁸²⁾Downs-Kelly, E., Yoder, B. J., Stoler, M., Tubbs, R. R., Skacel, M., Grogan, T., Roche, P., and Hicks, D. G. The influence of polysomy 17 on HER2 gene and protein expression in adenocarcinoma of the breast: a fluorescent in situ hybridization, immunohistochemical, and isotopic mRNA in situ hybridization study, Am. J. Surg. Pathol. 29: 12214227, 2005.
⁽⁸³⁾Pusztaszeri, M., Chaubert, P., Seelentag, W., and Bosman, F. T. Immunohistochemical Expression of Endothelial MARKERS CD31, CD34, von Willebrand Factor, and Fli-1 in Normal Human Tissues, JHC exPRESS doi:10.1369/jhc.4A6514.2005, 2005.
⁽⁸⁴⁾Shi, S. R., Cote, R. J., Yang, C., Chen, C., Xu, H. J., Benedict, W. F., and Taylor, C. R. Development of an optimal protocol for antigen retrieval: a ‘test battery’ approach exemplified with reference to the staining of retinoblastoma protein (pRB) in formalin-fixed paraffin sections, J. Pathol. 179: 347-352, 1996.
⁽⁸⁵⁾Shi, S.-R., Gu, J., Kalra, K. L., Chen, T., Cote, R. J., and Taylor, C. R. Chapter 1. Antigen retrieval technique: a novel approach to immunohistochemistry on routinely processed tissue sections. In: J. Gu (ed.) Analytical Morphology, Theory, Applications & Protocols, 1st edition, pp. 1-40. Natick, Mass., USA: Eaton Publishing Co., 1997.
⁽⁸⁶⁾Berger, A. J., Camp, R. L., Divito, K. A., Kluger, H. M., Halaban, R., and Rimm, D. L. Automated quantitative analysis of HDM2 expression in malignant melanoma shows association with early-stage disease and improved outcome, Cancer Res. 64: 8767-8772, 2004.
⁽⁸⁷⁾Kaur, R. and Raje, M. A solid-phase method for evaluation of gold conjugate used in quantitative detection of antigen by immunogold-labeling electron microscopy, J. Immunol. Methods. 279: 33-40, 2003.
⁽⁸⁸⁾Ramandeepa, Dikshita, K. L., and Rajea, M. Optimization of Immunogold Labeling TEM: An ELISA-based Method for Rapid and Convenient Simulation of Processing Conditions for Quantitative Detection of Antigen, J. Histochem. Cytochem. 49: 355-368, 2001.
⁽⁸⁹⁾Petersen, B. L., Sorensen, M. C., Pedersen, S., and Rasmussen, M. Fluorescence in situ hybridization on formalin-fixed and paraffin-embedded tissue: optimizing the method., Appl. Immunohistochem. Mol. Morphol. 12: 259-265, 2004.
⁽⁹⁰⁾Robinsona, J. M., Takizawab, T., and D., V. D. Enhanced Labeling Efficiency Using Ultrasmall Immunogold Probes: Immunocytochemistry, J. Histochem. Cytochem. 48:487-492, 2000.
⁽⁹¹⁾Tubbs, R., Pettay, J., Skacel, M., Powell, R., Stoler, M., Roche, P., and Hainfeld, J. Gold-facilitated in situ hybridization: a bright-field autometallographic alternative to fluorescence in situ hybridization for detection of Her-2/neu gene amplification, Am. J. Pathol. 160: 1589-1595, 2002.
⁽⁹²⁾Chaiwun, B., Shi, S.-R., Cote, R. J., and Taylor, C. R. Chapter 2. Major factors influencing the effectiveness of antigen retrieval immunohistochemistry. In: S.-R. Shi, J. Gu, and C. R. Taylor (eds.), Antigen Retrieval Techniques: Immunohistochemistry and Molecular Morphology, pp. 41-53. Natick, Mass.: Eaton Publishing, 2000.
⁽⁹³⁾Shi, S. R., Imam, S. A., Young, L., Cote, R. J., and Taylor, C. R. Antigen retrieval immunohistochemistry under the influence of pH using monoclonal antibodies, J. Histochem. Cytochem. 43: 193-201, 1995.
⁽⁹⁴⁾Taylor, C. R, Quantitative Internal Reference Standards for Immunohistochemistry. The measurement of quantity by weight. Appl Immunohistochem Mal Morph. 14: 253-259, 2006.
⁽⁹⁵⁾Taylor, C. R. and Levenson, R. M. Quantification of immunohistochemistry—issues concerning methods, utility and semi-quantitative assessment. Expert Opinion. J Histopath (in press) 2006.
⁽⁹⁶⁾Shi, S. R., Cheng, L. and Taylor, C. R. Standardization of Immunohistochemistry for Formalin-Fixed Paraffin-Embedded Tissue Sections Based on the Antigen Retrieval technique; from Experiments to Hypothesis. J Histochem Cyto Chem (in press) 2006.
⁽⁹⁷⁾Taylor, C. R. Standardization in Immunohistochemistry: the role of antigen retrieval in Molecular Morphology. Biotechnic and Histochem. 8:3-12, 2006.

### Example IV

### Consideration of the ASCO/CAP Task-Force Guideline Recommendations for Her2 Testing

### Background

The recently released ASCO/CAP Task-Force Guideline Recommendations, published simultaneously in the Journal of Clinical Oncology (1) and Archives of Pathology and Laboratory Medicine (2), address issues relevant to improving the accuracy of HER2 testing in breast cancer. These recommendations can be summarized as follows:

1. These recommendations will become mandatory requirements on Jan. 1, 2008 to all CAP-certified laboratories.

2. Testing algorithms were established for both IHC and FISH. The report includes a statement recognizing that HER2 “test results represent a continuous rather than a categoric variable”, i.e., these results simply can no longer be reported as binary. The Task Force, for the first time, recognizes that an “equivocal” gray zone exists, containing tumors with borderline scores of both IHC and FISH assays. Equivocal IHC samples (2+ score) must be confirmed by FISH analysis of the sample. Equivocal FISH samples are to be confirmed by counting additional cells or repeating the FISH test. If the FISH results remain equivocal, confirmatory IHC testing should be performed. “Equivocal” for FISH is defined by the Task Force as “moderate or weak complete staining in 10-30% of tumor cells or complete, non-uniform staining in >10% of cells.

3. By 2008, all CAP-accredited pathology laboratories performing HER2 testing must have validated their HER2 assay against either a different validated in-house assay or a validated similar assay done by another laboratory. A minimum of 25 invasive breast cancers is required. Practically speaking, if a pathology laboratory offers HER2 testing by IHC, it must validate its assay using results from another laboratory that has an established, clinically validated IHC assay. The same requirement applies for laboratories that offer both IHC and FISH assays, neither of which is clinically validated; a laboratory can only validate an assay internally, against another assay, if the other assay is itself clinically validated.

4. Importantly the guidelines also include a requirement that pathology laboratories must ensure that all breast excision specimens subject to HER2 testing are fixed in 10% neutral buffered formalin for 6-48 hours, and that core biopsies are fixed for at least 1 hour. Any and all alternative fixatives must be validated to ensure satisfactory “performance against the results of testing of the same samples fixed also in buffered formalin and tested with the identical HER2 assay, and concordance in this situation must also be 95%”.

5. The Task Force also raised the bar for the positive cutoff for the percentage of cells with 3+ score, from the previously FDA-approved 10% cutoff to a new 30% cutoff. The underlying rationale is that “very rarely . . . invasive tumors can show intense [3+], complete membrane staining of 30% or fewer tumor cells”.

6. Also for the first time, the Task Force accepts the fact that there is no gold standard assay for HER2 in breast cancer, not FISH and not IHC. While FISH technique has been viewed as a gold standard by some, evidence-based data do not confirm that notion.

7. Intrinsic to these guidelines is the acceptance that “no assay currently available is perfectly accurate to identify all patients expected to benefit or not from anti-HER2 therapy”. In other words, when we measure and achieve 95% concordance between two assays, we are not measuring the predictive value of each assay; merely that they are concordant.

8. New test rejection criteria were also established, and summarized in Tables 1 and 2, for IHC and FISH respectively.

9. These recommendations will undoubtedly undergo periodic reviews by the Task Force with expected revisions.

10. While the guidelines represent an important ‘leap forward’, some unresolved issues remain.

### Items Requiring Further Clarification:

The guidelines are for the most part specific and of real practical value. Nonetheless, in the opinion of the authors points for clarification include:

1. Test validation must be done “before offering the test clinically”. In reality, a good fraction of pathology laboratories in the US have been offering HER2 testing for clinical use prior to publication of these guidelines. The Task Force did not specify any concrete steps for these labs to validate the test retroactively; possibly the best that can be achieved is for all laboratories intending to offer either IHC or FISH HER2 assays to be in compliance by January 2008. The alternative is to cease testing.

2. The Task Force does not specify how the competency of the pathologists interpreting HER2 testing should be measured and monitored, particularly with regard to the reproducibility of scoring by both the IHC and FISH methods. Will an expanded CAP external evaluation program be available to meet this need? The UK NEQAS model (3) surely is the best available, requiring central consensus value reading of specific sample sections by experienced pathologists. Such a system may be hard to replicate in the larger diverse environment of the US, and who will pay for the costs of achieving this new better assay? Absent appropriate reimbursement success may be long coming.

3. There is no practical strategy in place for ensuring that specimens have been properly fixed; a minimum requirement would seem to be that the times of placement and removal of the tissue/biopsy into and from 10% formalin should be recorded (vide infra).

4. Then there is the practical problem in studies, and especially in clinical trials, of integrating the results of the ‘new improved’ guideline compliant test result, with the old. Going forward the decision is made for us by the mandate; but uncertainties will exist with regard to patients currently on, or not on, Herceptin therapy, especially those with equivocal tumors, and simple repeat testing will not necessarily solve the problem in the face on unknown tissue fixation conditions.

### Items Requiring Modification:

It is agreed that most breast cancers that are positive (3+) for HER2 over expression by IHC, give a quite uniform positive result across the tumor section, and in practice it is uncommon that the positive signal is patchy, or observed in <50% of cells (4). Nonetheless tumors do exist, albeit rarely, where there is clear and definite positive reaction (both by IHC and FISH) in a fraction (‘clone’) of tumor cells that overall averages much less than 30%. By the proposed guidelines, these tumors would be classified as negative. Most tumor biologists would concur that the HER2 positive tumor clone is likely to be more aggressive (than the HER2 negative component) and will ultimately dictate the biologic and clinical behavior of the tumor. Further consideration should be given as to whether such focally 3+ tumors should be classified as at least as equivocal, if not as positive.

The guidelines correctly imposed stringent requirements for the 6-48 hour-fixation window on excision specimens (lumpectomies, mastectomies); however, based on these guidelines recommendations, core biopsies require only a minimum of one-hour fixation. While formalin infiltration through the entire core biopsy may be effected within 1 hour, formalin is a very slow fixative and infiltration is not equivalent to fixation (4). We believe that the minimum fixation-time requirements for core biopsies should be as much as 6 hours, instead of one hour and that data exist to support this contention (5). Certainly we are not aware of convincing data that one hour fixation is sufficient. Ensuring the propriety of this fixation guideline is particularly important given that an increasing number of pathology laboratories are already performing HER2 IHC testing on the core biopsy rather than the excision specimen. It may be that a 6 hour fixation will preclude meeting the ‘requirements’ of our clinical colleagues in some situations; however, in the context of these new guidelines, reliable performance should govern practice, rather than expediency. Some have argued, with justification, that pressure from our clinical colleagues for the patient's results ‘yesterday’, has driven the use of abbreviated and unproven ‘rapid fixation’ protocols. If so, it is remarkable that now these same clinical colleagues are the major driving force behind recognition of the overriding necessity for improving the reliability of the HER2 assays, and we should thank them for it. In the final analysis the patient is likely to benefit from the right result, rather than the rapid one, and informed of the choice the patient undoubtedly would tell us that we need ‘to do it right’.

### CONCLUSION

We applaud and endorse the work of the ASCO/CAP Task Force. It is long awaited, and it is here; so we all need to deal with it. Perhaps the two items that have the biggest impact on pathology laboratories overall are tissue handling requirement and test monitoring requirements. Now the largest regulatory body in US pathology is finally recognizing that pathologists have been inflicting unknown and unknowable damage on specimens by not following proper fixation procedures. There are sufficient data to confirm that inadequate tissue fixation is responsible in large part for many of the reportedly false-negative results in hormone receptors testing in breast cancer (6, 7).

But HER2 is just the beginning. The growing list of ‘tests’ of critical prognostic/predictive markers that are being introduced into anatomic pathology makes this task of proper tissue fixation one of the most important ingredients of standardizing these tests, and represents a first essential step in converting these ‘stains’ into reliable assays. The high standards of quality control testing that have long been employed in the clinical pathology laboratory must be applied to tests that we perform across the hallway in the anatomic pathology laboratory. After all, isn't the IHC test a slightly modified version of the ELISA test? (8). For the results of any prognostic/predictive test to be clinically meaningful, rigorous quality control measures must be applied and followed, and we cannot avoid beginning at the beginning with proper specimen acquisition and handling protocols. The good news for anatomic pathology laboratories is we do know what needs to be done, and these measures aren't that difficult to implement.

### REFERENCES

1. Wolff A C, Hammond E H, Schwartz J N, et al. American Society of Clinical Oncology I College of American Pathologists Guideline Recommendations for Human Epidermal Growth Factor Receptor 2 Testing in Breast Cancer. J Clin Oncol 2007; 25(1):118-145.
2. Wolff A C, Hammond E H, Schwartz J N, et al. American Society of Clinical Oncology/College of American Pathologists Guideline Recommendations for Human Epidermal Growth Factor Receptor 2 Testing in Breast Cancer. Arch Pathol Lab Med 2007; 131(1):18-43.
3. Rhodes A, Jasani B, Anderson E, Dodson A R, Balaton A J. Evaluation of HER-2/neu Immunohistochemistry Assay Sensitivity and scoring on formalin-fixed and paraffin-processed cell lines and breast tumors: a comparative study involving results from laboratories in 21 countries. Am J Clin Pathol 2002; 118(3):408-417.
4. Fox C H, et al. Formaldehyde Fixation. J Histochem Cytochem 1985; 33:845-853.
5. Goldstein N S, Ferkowicz M, Odish E, et al. Minimum formalin fixation time for consistent estrogen receptor immunohistochemical staining of invasive breast carcinoma. Am J Clin Pathol. 2003; 120(1), 86-92.
6. Nadji M, Gomez-Fernandez C, Ganjei-Azar P, et al. Immunohistochemistry of estrogen and progesterone receptors reconsidered: experience with 5,993 breast cancers. Am J Clin Pathol 2005; 123(1):21-27.
7. Yaziji H, Goldstein L C, Barry T S, et al. HER-2 Testing in Breast Cancer Using Parallel Tissue-Based Methods. JAMA 2004; 29(16):1972-1977.
8. Taylor C R, Quantifiable Internal Reference Standards for Immunohistochemistry. The Measurement of Quantity by Weight. Appl Immunohistochem Mol Morphology 2007; 14.253-259.

### Example V

Over several decades immunohistochemistry has evolved from a methodologic curiosity, of occasional research interest, to a technique that is in widespread use in surgical pathology, and is considered to be essential in many areas of cancer diagnosis and classification. Today, there is a resurgent interest in assuring the reproducibility of the method, even to the point of upgrading it from a “stain” to a tissue-based “immunologic assay.” If accomplished, this change would make possible true quantification of analytes in tissue sections, analogous to the use of the enzyme-linked immunosorbent assay method in the clinical laboratory, which employs essentially the same reagents and similar principles, but is subject to much more rigorous control et all levels. (2,3)

Immunohistochemistry gives a tinctorial reaction that is readily viewed by routine light microscopy, leading pathologists to categorize the result as nothing more than a novel “special stain,” akin to a trichrome stain or a periodic acid-Schiff stain. The introduction of the hybridoma method 4 yielded a bounty of new antibodies, dozens of new “stains,” a burgeoning crop of new investigators, innovative variants of the method, new commercial vendors, easy to use “staining kits,” and even “automated stainers.” Over the last 2 decades the growth of literature in the field was explosive; it was an exciting time. One unintended consequence was that immunohistochemical stains were performed with beguiling ease in growing number of laboratories, with minimal attention to specimen acquisition, sample preparation (fixation), protocol, and controls, following a “modus operandi” that for more than a century had sufficed in the histopathology laboratory for an hematoxylin and eosin stain. As a result reproducibility suffered.

From the very beginning of immunoperoxidase-based studies, describing the immunohistochemical demonstration and distribution of various “antigens” in formalin-fixed tissues, findings were quite readily reproduced by other investigators; to be precise, they were reproduced, but they were not strictly reproducible. Thus, a tinctorial reaction (stain) might be reproduced by different investigators, but the intensity, distribution, and overall quality were inconsistent, from laboratory to laboratory, from day to day, from tissue to tissue within the same laboratory, and even in different regions of a single tissue section. This observed variability was attributed to uncertain quality of the primary antibody (from the same or different sources), to vagaries of technique, the aptitude or ineptitude of the investigator, or to differences in fixation, or lack thereof.

A number of workshops were convened over the years to examine these issues. The Biologic Stain Commission, working with the Food and Drug Administration, sponsored a series of conferences for investigators and manufacturers, at a number of which the author was privileged to be present, as the proverbial fly on the wall, and scribe. One tangible result was a major improvement in the validation and description of primary and labeling antibodies by manufacturers, culminating in more complete and uniform product labeling, incorporated into a comprehensive “package insert.” (5) A second outcome was the realization that, to improve the reproducibility of an “immunohistochemical stain,” the anatomic pathology laboratory must begin to adopt the standards and the “standardized” procedures of the clinical pathology laboratory. This notion was expressed under the tenet of the “Total Test,” (6) which advocated that the performing laboratory assume responsibility for all steps of the immunohistochemical procedure, from specimen acquisition, through sample preparation, fixation, processing, reagent validation, staining, and interpretation, specifically including the proper use of controls.

For a period in the 1980s, the effects of formalin fixation, for good or for ill, had held center stage. Frozen section methods were championed for a few short years, but never could overcome the poor morphologic detail inherent to this approach. Different fixatives were explored with little real success, and attention shifted to efforts intended to minimize the adverse effects of formalin fixation. Enzyme digestion methods yielded dramatic improvement in “staining” intensity in the hands of some investigators, but scarcely improved the reproducibility of immunohistochemistry as a whole. The introduction of “antigen retrieval” (7) (review Ref. 8) changed everything. Antigens that hitherto could not be stained in formalin paraffin sections, now stained; antibodies that did not work on fixed tissues now gave clear staining reactions, in even the least experienced hands. Overnight, pathologists could perform several hundred immunohistochemical “stains” on formalin paraffin sections. But there was another unintended consequence. With the effectiveness of retrieval methods pathologists concluded that they no longer needed to be overly concerned with fixation, so they were not, and once more fixation was ignored.

This state of affairs remained unchallenged for a number of years, for as long as immunohistochemical methods were employed simply as “stains” of lineage related markers of different cell types and their corresponding neoplasms. However, in the offing there was a new driver of change. In the mid-1990s estrogen and progesterone receptor analyses were adapted to the formalin paraffin tissue sections, superseding earlier cytosol-based methods. The effect was to create a new application of immunohistochemistry, namely the demonstration of prognostic and predictive markers. Suddenly, there were increased demands for reproducibility of immunohistochemical “stains,” to the point that quantification of expression levels of prognostic markers might be possible; that is measurement of actual amounts of protein within cells. In effect, the requirement was that the immunohistochemical stain should be upgraded from a simple qualitative “stain,” to a tissue-based, quantitative, immunologic assay, with all of the stringency thereby implied. It no longer sufficed to demonstrate that a particular marker (e.g., keratin, or CD20) was present (or absent) by the observation of staining (or lack thereof); the question became one of a higher order—exactly how much of the marker (read analyte) was present? Initial scoring methods for estrogen and progesterone receptor were at best semiquantitative, and were difficult to reproduce, in part because of inconsistency among different observers, but more critically because the underlying immunohistochemical staining process was inherently flawed. Experts reconvened, parallels were drawn once more with quantitative immunologic assays (enzyme-linked immunosorbent assay) in the clinical laboratory, and the “Total Test” approach for immunohistochemical stains was resurrected. In effect the debate over the desirability of standardization was over, the reality of rigorous test performance had arrived. (2,3) This time around there was a consensus that the inherent poor reproducibility had 2 major causes. First, specimen acquisition and sample preparation, including fixation, was entirely uncontrolled and highly variable within, and among, institutions. Second, although “in house” tissue controls were in use, there was a lack of suitable universal controls to assure reliability and reproducibility among different laboratories, and there were no quantifiable reference standards to provide a basis for accurate measurement of analytes.

Additional impetus and urgency arose from the realization that awareness of the poor reproducibility of immunohistochemical methods for the first time extended beyond the pathology community. Thus, colleagues in basic and clinical research voiced frustration upon encountering great variability of results for “tests” such as Her2 expression, which were considered critical for entry into certain clinical trials. This frustration found overt expression in recent requests for proposals from the NTH for studies of sample preparation, in the context of improving the reliability of molecular assays of cancerous tissues. (9) Pathologists around the globe have developed external quality control systems (UKNEQAS, CAP, referenced in the Report 1), that have resulted in demonstrable improvements in quality assurance of the staining method, but cannot address the adequacy or otherwise of sample preparation and fixation. At the time of writing new guidelines for the practice of immunohistochemistry are being formulated (Clinical Laboratory Standards Institute and College of American pathologists), to replace those existing, (10) but these large organizations by their very nature are somewhat deliberate in thought and action.

### REFERENCES

1. Goldstein N S, Hewitt S M, Taylor C R, et al, Members of Ad-Hoc Committee On Immunohistochemistry Standardization. Recommendations for improved standardization of immunohistochemistry. Appl Immunohistchem Mol. Morph. 2007.
2. Taylor C R. Quantifiable internal reference standards for immunohistochemistry: the measurement of quantity by weight. Appl Immunohistochem Mol. Morphol. 2006; 14:253-259.
3. Taylor C R, Levenson R M. Quantification of immunohistochemistry—issues concerning methods, utility and semiquantitative assessment II. Histopathology. 2006; 49:411-424.
4. Kohler G, Milstein C. Continuous cultures of fused cells secreting antibody of predefined specificity. Nature. 1975; 256:495-497.
5. Taylor C R. Report of the Immunohistochemistry Steering Committee of the Biological Stain Commission. “Proposed format: package insert for immunohistochemistry products.” Biotech Histochem. 1992; 67:323-338.
6. Taylor C R. Quality assurance and standardization in immunohistochemistry. A proposal for the annual meeting of the Biological Stain Commission, June, 1991. Biotech Histochem. 1992; 67:110-117.
7. Shi S R, Key M E, Kalra K L. Antigen retrieval in formalin fixed paraffin embedded tissues. J Histochem Cytochem. 1991; 39:741-748.
8. Shi S R, Cote R J, Taylor C R. Antigen retrieval immunohistochemistry and molecular morphology in the year 2001. Appl Immunohistochem Mol. Morphol. 2001; 9:107-116.
9. National Institutes of Health. RFA-CA-07-003. Innovations in Cancer Sample Preparation, US, National Cancer Institute, 2006.
10. O'Leary T J, Edmonds P, Floyd A D, et al. Quality Assurance for Immunocytochemistry: Approved Guidelines. Wayne PA NCCLS (now www CLSI), 1999.

What is claimed is:

1. A method of quantitatively determining the amount of a test analyte by IHC, comprising:
providing a formalin-fixed, paraffin-embedded (FFPE) cell or tissue sample comprising the test analyte, the FFPE sample having been prepared from an original cell or tissue sample having an original amount the test analyte at a collection time, T₁;
identifying a quantifiable internal reference standard (QIRS) for the test analyte, the QIRS being a second analyte present in the original cell or tissue sample at the collection time, T₁, and that is different from the test analyte;
providing one or more ratios consisting of the ratio of the amount of the test analyte to the amount of the QIRS in the original cell or tissue sample (A), the ratio of the amount of the test analyte to the amount of the QIRS in the FFPE sample (B), and the ratio of the amount of the QIRS in the original cell or tissue sample to the amount of the QIRS in the FFPE sample (C), said ratios being operable at a test time, T₂, after the collection time;
Generating an IHC signal corresponding to amount of QIRS in the test sample at the test time, T2;
generating an IHC signal corresponding to amount of test analyte in the test sample at the test time, T2; and
Calculating at least one of the amount of the test analyte in the test FFPE sample by multiplying the amount of the QIRS in the test FFPE sample by the ratio (B), and the amount of the test analyte in the test original cell or tissue sample by multiplying the amount of the QIRS in the test FFPE sample by the ratio (C) and by the ratio (A).

2. A method of determining the amount of a test antigen by IHC, comprising:
providing a formalin-fixed, paraffin-embedded (FFPE) cell or tissue sample comprising the test analyte, the FFPE sample having been prepared from an original cell or tissue sample having an original amount the test analyte at a collection time, T₁;
identifying a quantifiable internal reference standard (QIRS) for the test analyte, the QIRS being a second analyte present in the original cell or tissue sample at the collection time, T₁, and that is different from the test analyte;
providing a reference calibration curve indicating at least a ratio of the amount of the test antigen to the amount of the QIRS in a reference FFPE sample at test times, T₂, after T₁;
measuring a first IHC signal corresponding to the amount of the QIRS in the FFPE sample at test time T₂, wherein the first IHC signal varies depending on at least the concentration of the QIRS;
measuring a second IHC signal corresponding to the amount of the test analyte in the FFPE sample at time T₂, wherein the second IHC signal varies depending on at least the concentration of the test analyte; and
applying the calibration curve to the first IHC signal and the second IHC signal of the test antigen in the FFPE sample to determine the amount of the test antigen in the FFPE sample.

3. The method of claim 2, wherein the calibration curve provides a ratio, A, of the original amount of the test analyte to the original amount of the QIRS and a ratio, C, of the original amount of the QIRS to the amount of the QIRS in the FFPE sample at time T₂ is known, and the amount the test analyte in the original sample is calculated by multiplying the amount QIRS in the FFPE sample by the ratio A and by the Ratio C.

4. The method of claim 2, wherein the original cell is an endothelial cell or the original tissue contains endothelial cells, or the original cell is a lymphocyte or the tissue contains lymphocytes, or the original cell is a mesenchymal or epithelial cell, or the original tissue contains mesenchymal or epithelial cells.

5. The method of claim 2, wherein the QIRS is selected from the group consisting of CD31, actin, B2 microglobulin, vimentin, factor VIII, histone H1, MIB1, Fli 1, CD34, and VWF.

6. The method of claim 2, wherein the QIRS is a cell surface protein, a cytoplasmic protein, or a nuclear protein.
